(19) United States
(12) Patent Application Publication
(10) Pub. No.: US 2020/0148682 A1
(43) Pub. Date: May 14, 2020

(54) AKT ISOZYME-SPECIFIC COVALENT INHIBITORS DERIVED FROM REDOX-SIGNALING LIPIDS

(71) Applicant: Cornell University, Ithaca, NY (US)

(72) Inventors: Yimon AYE, Ithaca, NY (US); Xuyu LIU, Ithaca, NY (US); Marcus J.C. LONG, Ithaca, NY (US)

(21) Appl. No.: 16/619,245

(22) PCT Filed: Jun. 6, 2018
(86) PCT No.: PCT/US2018/036210

§ 371 Date: Dec. 4, 2019

Related U.S. Application Data
(60) Provisional application No. 62/515,984, filed on Jun. 6, 2017.

(51) Int. Cl. C07D 471/14; A61P 35/00

(52) U.S. Cl. CPC C07D 471/14; A61P 35/00

(57) ABSTRACT

The present invention is directed to a compound of Formula (I) wherein A, X, Y, R¹, R², R³, R⁴, and n are as described herein. The present invention also relates to: 1) a method of treating cancer using a compound of Formula (I) and 2) a method of inhibiting pan-Akt in a cell or a tissue using a compound of Formula (I).

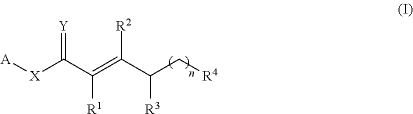

This application claims the priority benefit of U.S. Provisional Patent Application Ser. No. 62/515,984, filed Jun. 6, 2017, which is hereby incorporated by reference in its entirety.

This invention was made with government support under GM114850 awarded by National Institutes of Health; 1351400 awarded by the National Science Foundation; and N00014-17-1-2529 awarded by the Office of Naval Research. The government has certain rights in the invention.

### FIELD OF THE INVENTION

The present invention relates to Akt isozyme-specific covalent inhibitors derived from redox-signaling lipids.

### BACKGROUND OF THE INVENTION

Since its early inception in the mid 1800's to the huge industrialization efforts in the 20^{th} century, the story of drug discovery is one of considerable success driven by enormous innovative efforts. Over the course of this iterative process, a huge knowledge-bank has accumulated, much of which has been encapsulated in classic rules, such as “the rule of five” (Lipinski, “Lead- and Drug-Like Compounds: The Rule-Of-Five Revolution,” Drug Discovery Today: Technologies 1(4):337-341 (2004)) and Pfeiffer's rule (Barlow, “Enantiomers: How Valid is Pfeiffer's rule?,” Trends Pharmacol. Sci. 11(4):148-150 (1990)). However, in spite of our apparent understanding of the drug discovery process, there is little tangible evidence that we are getting better at designing drugs: overall the number of drugs failing trials, especially in oncology, has mostly increased year on year (Wong et al., “Estimation of Clinical Trial Success Rates and Related Parameters,” Biostatistics kxx069 (2018)); the number of validated drug targets has not hugely increased and it is still only able to target a small fraction of the human genome (Backus et al., “Proteome-Wide Covalent Ligand Discovery in Native Biological Systems,” Nature 534:570-574 (2016)). In line with the deep-rooted traditions of innovation, several visionaries have pioneered methods to improve the properties of drugs and allow opening up of the playing field to encompass more nontraditional drug targets. These include: degradation-inducing drugs (Bondeson et al., “Lessons in PROTAC Design from Selective Degradation With a Promiscuous Warhead,” Cell Chemical Biology 25(1):78-87 (2018)), polypharmacological drugs (Reddy et al., “Polypharmacology: Drug Discovery for the Future,” Expert Rev. Clin. Pharmacol. 6(1):41-47 (2013); Morphy et al., “From Magic Bullets to Designed Multiple Ligands,” Drug Discovery Today 9(15):641-651 (2004)), combination therapies (Mokhtari et al., “Combination Therapy in Combating Cancer,” Oncotarget 8(23):38022-38043 (2017)), and covalent drugs (Singh et al., “The Resurgence of Covalent Drugs,” Nat. Rev. Drug Discovery 10:307-317 (2011); Long et al., “Privileged Electrophile Sensors: A Resource for Covalent Drug Development,” Cell Chemical Biology 24(7):787-800 (2017)).

The drug industry has had a long and checkered history with covalent inhibitors; one of the primary and ongoing success stories of modern drug design is penicillin, a covalent inhibitor of the bacterial enzyme, DD-transpeptidase that contains a reactive β-lactam motif (Lobanovska et al., “Focus: Drug Development: Penicillin's Discovery and Antibiotic Resistance: Lessons for the Future?,” The Yale Journal of Biology and Medicine 90(1):135-145 (2017)); on the other hand, many reactive appendages can lead to haptenization and ultimately drug induced liver injuries (Singh et al., “The Resurgence of Covalent Drugs,” Nat. Rev. Drug Discovery 10:307-317 (2011); Stephens et al., “Mechanisms of Drug-Induced Liver Injury,” Current Opinion in Allergy and Clinical Immunology 14(4):286-292 (2014)). These mechanisms are hard to predict yet contribute to clinical trial failure, and can lead to death. On account of opinion presuming haptenization to be a common pathway, drug companies shied away from deliberately designing covalent inhibitors throughout the mid-late part of the 20^{th} century. However, throughout this time, several serendipitous covalent inhibitors arose and were successful in the clinic (Bauer, “Covalent Inhibitors in Drug Discovery: From Accidental Discoveries to Avoided Liabilities and Designed Therapies,” Drug Discovery Today 20(9): 1061-1073 (2015)). The realization that pharmacokinetic parameters can dominate drug efficacy has over the last 20-30 years led to a re-evaluation of the utility of covalent inhibitors. Because of this paradigm shift in critically appreciated drug parameters, several modern covalent inhibitors have been released. These include Afatinib and Dacomitinib. These drugs are clearly semi-rationally designed from a parent, non-covalent eGFR inhibitor, Gefitinib (Vokes et al., “EGFR-Directed Treatments in SCCHN,” Lancet Oncol. 14(8): 672-673 (2013); Keating, “Afatinib: a Review of its use in the Treatment of Advanced Non-Small Cell Lung Cancer,” Drugs 74(2):207-221 (2014); Dutton et al., “Gefitinib for Oesophageal Cancer Progressing After Chemotherapy (COG): a Phase 3, Multicentre, Double-Blind, Placebo-Controlled Randomised Trial,” Lancet Oncol. 15(8):894-904 (2014)).

Yet strategies to install covalent appendages into existing drugs are poorly developed. Furthermore, it is unclear how the nature of these appendages affect target engagement and/or target occupancy, parameters that profoundly impact clinical efficacy (Simon et al., “Determining Target Engagement in Living Systems,” Nat. Chem. Biol. 9:200-205 (2013)). In fact, it seems that most of the currently successful covalent drugs derived from non-covalent first generation strategies function through a “bolt”-like strategy (Tsai et al., “Selective, Rapid and Optically Switchable Regulation of Protein Function in Live Mammalian Cells,” Nat. Chem. 7:554-561 (2015)); i.e. the covalent ligand serves to permanently attach the ligand to the enzyme with little other benefit either kinetic (e.g., through targeting of an intrinsically hyper-reactive cysteine) or functional (e.g., through targeting a cysteine that has function in its own right). While success of covalent drugs targeting apparent spectator cysteines shows such a strategy is not necessary for drug design, it remains unknown what benefit(s) can by obtained by targeting cysteines with function/privilege (Long et al., “Privileged Electrophile Sensors: A Resource for Covalent Drug Development,” Cell Chemical Biology 24(7):787-800 (2017)).

The present invention is directed to overcoming these and other deficiencies in the art.

### SUMMARY OF THE INVENTION

One aspect of the present invention relates to a compound of Formula (I):

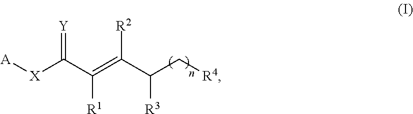

wherein

A is a reversible pan-Akt inhibitor or a radical thereof;

X is optional, and, if present, is NH, O, or S;

Y is O or S;

R¹ is H, halogen, NO₂, CN, C₁₋₆ alkyl, or aryl;

R² is H, halogen, NO₂, CN, C₁₋₆ alkyl, or aryl;

R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;

R⁵ is H, C₁₋₆ alkyl, or aryl;

R⁶ is H, C₁₋₆ alkyl, or aryl; and

n is 0, 1, 2, 3, 4, or 5;

or an oxide thereof, a pharmaceutically acceptable salt thereof, a solvate thereof, or a prodrug thereof.

Another aspect of the present invention relates to a method of treating cancer. This method includes administering to a subject a compound of the Formula (I):

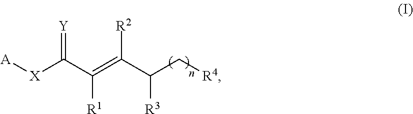

wherein

A is a reversible pan-Akt inhibitor or a radical thereof;

X is optional, and, if present, is NH, O, or S;

Y is O or S;

R¹ is H, halogen, NO₂, CN, C₁₋₆ alkyl, or aryl;

R² is H, halogen, NO₂, CN, C₁₋₆ alkyl, or aryl;

R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;

R⁵ is H, C₁₋₆ alkyl, or aryl;

R⁶ is H, C₁₋₆ alkyl, or aryl; and

n is 0, 1, 2, 3, 4, or 5;

or an oxide thereof, a pharmaceutically acceptable salt thereof, a solvate thereof, or a prodrug thereof,
under conditions effective to treat cancer in the subject.

Another aspect of the present invention relates to a method of inhibiting pan-Akt in a cell or a tissue. This method includes providing a compound of Formula (I):

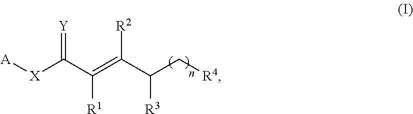

wherein

A is a reversible pan-Akt inhibitor or a radical thereof;

X is optional, and, if present, is NH, O, or S;

Y is O or S;

R¹ is H, halogen, NO₂, CN, C₁₋₆ alkyl, or aryl;

R² is H, halogen, NO₂, CN, C₁₋₆ alkyl, or aryl;

R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;

R⁵ is H, C₁₋₆ alkyl, or aryl;

R⁶ is H, C₁₋₆ alkyl, or aryl; and

n is 0, 1, 2, 3, 4, or 5;

or an oxide thereof, a pharmaceutically acceptable salt thereof, a solvate thereof, or a prodrug thereof; and

contacting a cell or tissue with the compound under conditions effective to inhibit pan-Akt.

A class of cysteine that exists in a diverse range of proteins has been disclosed. These include enzymes (Akt3 (Long et al., “Akt3 is a Privileged First Responder in Isozyme-Specific Electrophile Response,” Nat. Chem. Biol. 13(3):333-338 (2017), which is hereby incorporated by reference in its entirety), Keap1(Lin et al., “A Generalizable Platform for Interrogating Target- and Signal-Specific Consequences of Electrophilic Modifications in Redox-Dependent Cell Signaling,” J. Am. Chem. Soc. 137(19):6232-6244 (2015); Long et al., “β-TrCP1 Is a Vacillatory Regulator of Wnt Signaling,” Cell Chemical Biology 24(8):944-957.e7 (2017); Fang et al., “Temporally Controlled Targeting of 4-Hydroxynonenal to Specific Proteins in Living Cells,” J. Am. Chem. Soc. 135(39):14496-14499 (2013); Parvez et al., “Substoichiometric Hydroxynonenylation of a Single Protein Recapitulates Whole-Cell-Stimulated Antioxidant Response,” J. Am. Chem. Soc. 137(1):10-13 (2015), which are hereby incorporated by reference in their entirety), PTEN (Fang et al., “Temporally Controlled Targeting of 4-Hydroxynonenal to Specific Proteins in Living Cells,” J Am. Chem. Soc. 135(39):14496-14499 (2013); Parvez et al., “T-REX on-Demand Redox Targeting in Live Cells,” Nat. Protoc. 11(12):2328-2356 (2016), which are hereby incorporated by reference in their entirety)), non-ATP dependent chaperones (HSPB7 (Surya et al., “Cardiovascular Small Heat Shock Protein HSPB7 Is a Kinetically Privileged Reactive Electrophilic Species (RES) Sensor,” ACS Chem. Biol. (2018), which is hereby incorporated by reference in its entirety)), and allosteric enzyme regulators (Ube2V2, Ube2V1(Zhao et al., “Ube2V2 Is a Rosetta Stone Bridging Redox and Ubiquitin Codes, Coordinating DNA Damage Responses,” ACS Cent. Sci. 4(2): 246-259 (2018), which is hereby incorporated by reference in its entirety)). These cysteines have several properties that could prove to be hugely important for drug design: they display rapid second order reaction kinetics with biological enones and enals; modification of these residues is intrinsically linked to function; they can elicit amplicative effects, such that one inhibition event can affect multiple downstream proteins (Long et al., “Privileged Electrophile Sensors: A Resource for Covalent Drug Development,” Cell Chemical Biology 24(7):787-800 (2017); Long et al., “The Die Is Cast: Precision Electrophilic Modifications Contribute to Cellular Decision Making,” Chem. Res. Toxicol. 29(10):1575-1582 (2016); Poganik et al., “Getting the Message? Native Reactive Electrophiles Pass Two Out of Three Thresholds to be Bona Fide Signaling Mediators,” BioEssays 40(5): 1700240 (2018), which are hereby incorporated by reference in their entirety). A novel assay that is able to uncover these privileged cysteines in a HT screen was released (Zhao et al., “Ube2V2 Is a Rosetta Stone Bridging Redox and Ubiquitin Codes, Coordinating DNA Damage Responses,” ACS Cent. Sci. 4(2): 246-259 (2018), which is hereby incorporated by reference in its entirety).

It was found that Akt3 and to a lesser extent Akt2 can be targeted for inhibition by hydroxynonenal: hydroxynonenal does not affect Akt1 (Long et al., “Akt3 is a Privileged First Responder in Isozyme-Specific Electrophile Response,” Nat. Chem. Biol. 13(3):333-338 (2017), which is hereby incorporated by reference in its entirety). These results are intriguing, because they seem to form a pathway to Akt-isoform-selective inhibition (Long et al., “Privileged Electrophile Sensors: A Resource for Covalent Drug Development,” Cell Chemical Biology 24(7):787-800 (2017), which is hereby incorporated by reference in its entirety). The druggability of the privileged cysteine residue within Akt3 was evaluated by evolving an Akt1-selective non-covalent inhibitor, to a covalent Akt3-selective analog. Such a strategy is of significant importance, because the different Akt isoforms behave antagonistically (Linnerth-Petrik et al., “Opposing Functions of Akt Isoforms in Lung Tumor Initiation and Progression,” PLOS ONE 9(4):e94595 (2014); Liu et al., “Mechanism of Akt1 Inhibition of Breast Cancer Cell Invasion Reveals a Protumorigenic Role for TSC2,” Proc. Nat'lAcad. Sci. USA 103(11):4134-4139 (2006), which are hereby incorporated by reference in their entirety). In particular, Akt1 maintains breast tumor stability and relays anti-migratory and anti-invasive signals (Liu et al., “Mechanism of Akt1 Inhibition of Breast Cancer Cell Invasion Reveals a Protumorigenic Role for TSC2,” Proc. Nat'l Acad. Sci. USA 103(11):4134-4139 (2006), which is hereby incorporated by reference in its entirety); whereas Akt2 and Akt3 promote breast tumor aggressiveness and initiate metastasis both in cell and in xenograft models (Dillon et al., “Distinct Biological Roles for the Akt Family in Mammary Tumor Progression,” Cancer Research 70(11):4260-4264 (2010); Chin et al., “Targeting Akt3 Signaling in Triple-Negative Breast Cancer,” Cancer Research 74(3):964-973 (2014); Chin et al., “PTEN-Deficient Tumors Depend on AKT2 for Maintenance and Survival,” Cancer Discovery 4(8): 942-955 (2014), which are hereby incorporated by reference in their entirety). However, current Akt inhibitors generally have poor isoform selectivity with a moderate preference for Akt1 inhibition. Akt3 is typically the least responsive isoform to these inhibitors (Stottrup et al., “Upregulation of AKT3 Confers Resistance to AKT Inhibitor MK2206 in Breast Cancer,” Molecular Cancer Therapeutics 15(8): 1964-1974 (2016), which is hereby incorporated by reference in its entirety).

The present application demonstrates that Michael acceptor appendages (α,β-unsaturated carbonyls that modify specific nucleophilic residues in proteins of interest via conjugate addition) that resemble the endogenous lipid signaling electrophile are best able to inhibit Akt3 selectively, even over more reactive analogs. It further shows that at least in the case of Akt3, properties of the resulting drug can be dominated by the enone moiety, not the original drug scaffold. This opens new avenues for isoform-selective drug design.

### BRIEF DESCRIPTION OF THE DRAWINGS

FIGS. 1A-1B show an ¹H NMR spectrum (FIG. 1A) and a ¹³C NMR spectrum (FIG. 1B) of ethyl (E)-4-((tert-butyldimethylsilyl)oxy)non-2-en-8-ynoate.

FIGS. 2A-2B show an ¹H NMR spectrum (FIG. 2A) and a ¹³C NMR spectrum (FIG. 2B) of (E)-4-((tert-butyldimethylsilyl)oxy)-N-(1-(4-(3-oxo-9-phenyl-2,3-dihydro-[1,2,4]triazolo[3,4-f][1,6]naphthyridin-8-yl)phenyl)cyclobutyl)non-2-en-8-ynamide.

FIGS. 3A-3B show an ¹H NMR spectrum (FIG. 3A) and a ¹³C NMR spectrum (FIG. 3B) of MK-HNE.

FIGS. 4A-4B show an ¹H NMR spectrum (FIG. 4A) and a ¹³C NMR spectrum (FIG. 4B) of (E)-4-((tert-butyldimethylsilyl)oxy)non-2-enoic acid.

FIGS. 5A-5B show an 1H NMR spectrum (FIG. 5A) and a ¹³C NMR spectrum (FIG. 5B) of (E)-4-((tert-butyldimethylsilyl)oxy)-N-(1-(4-(3-oxo-9-phenyl-2,3-dihydro-[1,2,4]triazolo[3,4-f][1,6]naphthyridin-8-yl)phenyl)cyclobutyl)non-2-enamide.

FIGS. 6A-6B show an ¹H NMR spectrum (FIG. 6A) and a ¹³C NMR spectrum (FIG. 6B) of (E)-8-(4-(1-(4-hydroxynon-2-enamido)cyclobutyl)phenyl)-3-oxo-9-phenyl-2,3-dihydro-[1,2,4]triazolo[3,4-f][1,6]naphthyridin-7-ium chloride (MK-HNE no alkyne).

FIGS. 7A-7B show an ¹H NMR spectrum (FIG. 7A) and a ¹³C NMR spectrum (FIG. 7B) of (E)-4-fluoronon-2-en-8-ynoic acid.

FIGS. 8A-8B show an ¹H NMR spectrum (FIG. 8A) and a ¹³C NMR spectrum (FIG. 8B) of MK-FNE.

FIGS. 9A-9B show an ¹H NMR spectrum (FIG. 9A) and a ¹³C NMR spectrum (FIG. 9B) of MK-dHNE.

FIGS. 10A-10B show an ¹H NMR spectrum (FIG. 10A) and a ¹³C NMR spectrum (FIG. 10B) of 4-((tert-butyldimethylsilyl)oxy)-N-(1-(4-(3-oxo-9-phenyl-2,3-dihydro-[1,2,4]triazolo[3,4-f][1,6]naphthyridin-8-yl)phenyl)cyclobutyl)non-8-ynamide.

FIGS. 11A-11B show an ¹H NMR spectrum (FIG. 11A) and a ¹³C NMR spectrum (FIG. 11B) of MK-HNA.

FIGS. 12A-12B show an ¹H NMR spectrum (FIG. 12A) and a ¹³C NMR spectrum (FIG. 12B) of (E)-8-(4-(1-(4-fluoronon-2-enamido)cyclobutyl)phenyl)-3-oxo-9-phenyl-2,3-dihydro-[1,2,4]triazolo[3,4-f][1,6]naphthyridin-7-ium chloride (MK-FNE no alkyne).

FIGS. 13A-13B show an ¹H NMR spectrum (FIG. 13A) and a ¹³C NMR spectrum (FIG. 13B) of 3-((tert-butyldimethylsilyl)oxy)-2-methylene-N-(1-(4-(3-oxo-9-phenyl-2,3-dihydro-[1,2,4]triazolo[3,4-f][1,6]naphthyridin-8-yl)phenyl)cyclobutyl)non-8-ynamide.

FIGS. 14A-14B show an ¹H NMR spectrum (FIG. 14A) and a ¹³C NMR spectrum (FIG. 14B) of MK-G.

FIGS. 15A-B show the chemical structures of MK-G (FIG. 15A) and MK-HNE (FIG. 15B).

FIGS. 16A-16D show that MK-HNE exhibits Akt isoform-specific covalent binding through targeting redox-sensing cysteine residues on Akt2 and Akt3(C119), respectively. FIG. 16A shows that Akt2 and Akt3(WT) are privileged targets for MK-HNE covalent association. HEK293T cells were transfected with His6-Halo-Akt1-2xFlag, His6-Halo-Akt2-2xFlag, His6-Halo-Akt3C119S-2xFlag, or His6-Halo-Akt3-2xFlag followed by either 5 μM MK-HNE (alkyne-functionalized) or DMSO in 3% FBS treatment for 48 hours before lysis. Overexpressed Akt proteins were labeled with Cy5-small-molecule-fluorophore-conjugated azide (Cy5-azide) using standard click chemistry. FIG. 16A top gel is an image showing Cy5 channel (used to evaluate the extent of covalent binding on each Akt protein). The lysate from non-transfected HEK 293T cells was also used for comparison. M, molecular weight ladder. FIG. 16A (in the lower panels) is an image showing western blots probing Halo (upper part; detects ectopic Akt isoforms) and actin (lower part; loading control), respectively. FIG. 16B shows that MK-G does not exhibit Akt isoform-selective covalent inhibition. The transfected HEK293T cells were treated with either 5 μM MK-G or DMSO in 3% FBS for 48 hours before lysis. MK-HNE treatment samples were used for comparison. FIG. 16B top gel is an image showing Cy5 channel. M is a molecular weight ladder. FIG. 16B bottom is an image showing western blots probing Halo and actin, respectively. FIG. 16C is a graph showing quantification of Cy5 signal intensity on the band corresponding to POI after 48 hours of 5 μM MK-HNE treatment (3 biological replicates). FIG. 16D is a graph showing direct comparison between MK-G and MK-HNE treated Akt-transfected HEK293T cells based on Cy5 labeling. The Cy5 signal from compound (MK-HNE or MK-G)-treated samples were normalized by the signal intensity of the corresponding untreated samples and the abundance of Halo proteins. Error bars designate s.d. (MK-HNE treated samples, n=3; MK-G treated samples, n=2 independent biological replicates). Normalized fluorescent signal (NFS) was calculated from the following equation: NFS=[(Cy5_{(X μM drug)}−Cy5_{(No transfection)})/Halo_{(X μM drug)}]÷[(Cy5_{(no drug)}−Cy5_{(No transfection)})/Halo_{(no drug)}]. The relative fluorescent signal (RFS) is calculated from the following equation: RFS=(Cy5_{(X μM drug)}/Halo_{(X μM drug)}) (Cy5_{(no drug)}/Halo_{(no drug)})

FIGS. 17A-17D show dose- and time-dependent covalent association between Akt3 and MK-HNE. FIG. 17A top gel is an image showing western blot Cy5 channel. M is a molecular weight ladder. FIG. 17A lower panels are images showing western blots probing Halo (Akt3 expression; upper part) and actin (loading control; lower part) proteins, respectively. HEK293T cells were transfected with His6-Halo-Akt3-2xFlag followed by treatment with various final concentrations of MK-HNE (alkyne-functionalized) (0, 1, 2.5, 5, 10 and 25 μM) in 3% FBS for 24 hours before lysis. The corresponding cell lysates were labeled with Cy5-azide using standard click chemistry. FIG. 17B top gel is an image showing western blot Cy5 channel. M is a molecular weight ladder. FIG. 17B lower panels are images of western blots showing Halo and actin, respectively. Akt3-transfected HEK293T cells were incubated in 3% FBS with 5 μM MK-HNE(alkyne-functionalized) for various time periods (0, 5.5, 10, 18, 26, 36 and 48 hours). FIGS. 17C-17D are graphs showing quantification of relative Cy5 signal intensity on the band corresponding to drug concentration or treatment time (n=3, independent biological replicates). Relative fluorescent signal (RFS) is calculated from the following equation: RFS=(Cy5_{(X μM drug)}/Halo_{(X μM drug)})÷(Cy5_{(no drug)}/Halo_{(no drug)})

FIG. 18 is an image showing that MK-HNE labels endogenous Akts. Native HEK293T cells were treated with 5 μM MK-HNE(alkyne-functionalized) or an equivalent volume of DMSO in 3% FBS for 12 hours before lysis. A fraction of each sample was used for the input blot. The remaining cell lysates were labeled with biotin-azide using standard click chemistry followed by streptavidin enrichment. Western blot with anti-pan-Akt antibody demonstrates the enrichment of endogenous Akt protein(s) in cells treated with MK-HNE.

FIGS. 19A-19F show that MK-2206 functionalized with HNE-like appendages covalently labels Akt3. HEK293T cells were transfected with Halo-Akt3 for 20 hours, then treated with the stated compound (5 μM, 48 hours). After this time, cells were lysed and the labeling was assayed by click chemistry. MK-HNA is a derivative of MK-HNE that cannot interact covalently with Akt3. FIGS. 19A, 19C top gels are images showing in gel fluorescence, Cy5 channel. M is a molecular weight ladder. FIG. 19A, 19C lower panels are images of western blots (probing for Halo and actin, respectively) transferred from the same gel as the top image. FIG. 19B shows the chemical structure of MK-HNA. FIG. 19D shows the chemical structures of MK-HNE, MK-FNE, and MK-dHNE. FIGS. 19E-19F are graphs showing quantitation of in-gel Cy5 signal intensity, divided by the western blot signal for Halo.

FIGS. 20A-20H show the kinetic analysis of Akt3 covalent inhibition by MK-HNE, MK-FNE, and MK-dHNE. FIG. 20A is a schematic representation of the NADH-coupled kinetic assay to monitor the progress of Akt-catalyzed phosphorylation of the known peptide substrate, Crosstide (Akt substrate, cat. No. sc-471145, Santa Cruz). FIG. 20B shows a proposed kinetic mechanism that describes MK-HNE covalent inhibition. FIGS. 20C, 20E, and 20G are graphs showing inhibition of recombinant Akt3 (5 μM) by MK-HNE (FIG. 20C), MK-dHNE (FIG. 20E), and MK-FNE (FIG. 20G). The solid curves are the best nonlinear fits of the data using EQN. 1. FIG. 20D is a graph showing a replot of k_{obs} for the progress curve as a function of MK-HNE concentration. FIG. 20F is a graph showing a replot of k_{obs} for the progress curve as a function of MK-dHNE concentration. The hyperbolic curve is the best nonlinear fit to EQN. 2. FIG. 20H is a graph showing k_{obs} for the progress curve as a function of MK-FNE concentration fit to a cooperative binding equation using EQN 3. Inhibition of recombinant Akt3 (5 μM) by MK-HNE, MK-dHNE, and MK-FNE was assayed by progress curve analysis: inhibitor was added to enzyme in assay buffer containing 100 nM Akt3 kinase, 100 μM Crosstide (Akt substrate, cat. No. sc-471145, Santa Cruz), 500 μM ATP, 4 mM TCEP, 5 mM phosphoenolpyruvate, pyruvate kinase (24-40 units/mL), lactate dehydrogenase (36-56 units/mL), 500 μM NADH and various concentrations of the inhibitor. All curves were background rate subtracted. Product formation was measured continuously for 6 min. Over this time, Akt3 activity was constant in the absence of the inhibitors.

FIGS. 21A-21H show that MK-HNE irreversibly inhibits Akt3 in cells and that this process requires C119 residue (a known cysteine labeled by HNE ((Long et al., “Akt3 is a Privileged First Responder in Isozyme-Specific Electrophile Response,” Nat. Chem. Biol. 13(3):333-338 (2017), which is hereby incorporated by reference in its entirety)). FIG. 21A is a schematic showing the FRET assay used. The protein shown (that reports on Akt activity) is referred to as “AKTAR”. FIG. 21B is a schematic showing the time scales over which the Akt FRET assay was carried out and how drug withdrawal/cell recovery was investigated. FIGS. 21C-21D: HEK293T cells were transfected with plasmids encoding AKTAR and Halo-Akt3 (1:1). After 12 hours, cells were treated with MK-2206 (5 μM) or MK-HNE (5 μM). After 24 hours and 48 hours Akt3 activity was measured using ratiometric FRET analysis in live cells. After 48 hours, cells were washed two times with drug-free media containing 10% FBS and allowed to recover in 3% FBS MEM media for another 24 hours, after which time Akt3 activity was assayed using FRET again. wd=withdrawal for 24 hours. FIG. 21C shows representative images after 24-hour treatment of HEK293T cells expressing Halo-Akt3. FIG. 21D is a graph showing quantitation of all data sets for treatment of HEK293T cells expressing Halo-Akt3/AKTAR. FIG. 21E shows representative images after 24-hour treatment of HEK293T cells expressing Halo-Akt2/AKTAR. FIG. 21F is a graph showing quantitation of all data sets for treatment of HEK293T cells expressing Halo-Akt2/AKTAR. FIG. 21G shows representative images 24-hour treatment of HEK293T cells expressing Halo-Akt3C119S mutant/AKTAR. FIG. 21H is a graph showing quantitation of all data sets for treatment of HEK293T cells expressing Halo-Akt3C119S mutant/AKTAR.

FIG. 22A-22M show that MK-FNE is as potent as MK-2206 against TNBC lines. MK-FNE proliferation effects proceed through Akt3 inhibition. FIGS. 22A and 22D are graphs showing results of cytotoxicity assays for MDA-MB-468 (FIG. 22A) and BT-549 (FIG. 22D). FIGS. 22C and 22E show PI₅₀s calculated from the data shown in FIGS. 22A and 22D. FIGS. 22G and 22H are graphs showing results of cytotoxicity assays for MK-FNE in MDA-MB-468 cells treated with the stated siRNA (FIG. 22G) and MK-2206 in MDA-MB-468 cells treated with the stated siRNA (FIG. 22H). FIG. 22I shows EC₅₀s and fold selectivity for MK-FNE and MK-2206 calculated from the data shown in FIGS. 22G and 22 H. FIG. 22B shows the chemical structures of HNE alkyne. Stated cell lines were plated in 96-well plates (3000 cells per well) in the presence of the stated concentration of inhibitor. After 72 hours, the number of viable cells was analyzed by AlamarBlue®. Proliferation inhibition 50% (PI₅₀) values were calculated by fitting data to “PI₅₀ equation” (EQN 4) (FIG. 22C). MDA cells were transfected with the stated siRNA using lipofectamine 3000 for 12 hours. After this time, cells were plated in 96 well plates (3,000 cells per well) and settled for another 12 hours before adding differing concentrations of MK-FNE. After 72 hours, the number of viable cells was calculated using AlamarBlue®. FIG. 22F is a schematic showing AlamarBlue® viability assay. FIGS. 22J-22L show representative blots showing knockdown of the intended protein. FIG. 22M shows representative data displaying sorting of single cells by scatter properties and histogram of DNA content gated by phase. MDA-MB-468 cells were treated with the stated drugs for 72 hours at the PI₆₀ and PI₈₀. After this time, cells were harvested, fixed/permeabilized with ethanol and DNA content was stained using propidium iodide (visualized in the 488-3 channel). RNA was removed simultaneously using RNAse. After 30 min at room temperature, cells were analyzed by flow cytometry.

FIGS. 23A-231 show that the pharmaceutical programs of MK-HNE/MK-FNE are unexpectedly different from MK-2206. In particular, MK-FNE is ideally suited to targeting triple-negative breast cancers (TNBCs). FIG. 23A shows domain composition of Akt isoforms. Redox/electrophile-sensing cysteine residue of Akt2(C124) and Akt3(C119) are highlighted (Linker domain: Akt1 (EEEEMDFRSGSPSDNSG (SEQ ID NO: 1)); Akt2 (PGEDMDYKCGSPSDSST (SEQ ID NO: 2)); and Akt3 (EEERMNCSPTSQIDNIGE (SEQ ID NO: 3)). Phosphorylated T305 of Akt3 and the analogous phosphorylation sites in Akt1(T308) and Akt2(T309) are highlighted (Kinase domain: Akt1 (DGATMKTFCGTPEYLAPE (SEQ ID NO: 4)); Akt2 (DGATMKTFCGTPEYLAPE (SEQ ID NO: 5)); and Akt3 (DAATMKTFCGTPEYLAPE (SEQ ID NO: 6)). FIGS. 23B-23D show western blot data. HEK293T cells expressing the stated Halo-tagged Akt gene were treated with DMSO, MK-2206, or MK-HNE (5 μM, 48 hours). After this time, cells were lysed and analyzed by antibodies for phosphorylated T305 (pT305) of Akt3, Halo (Akt), and Actin (loading control). FIG. 23E is a graph showing the significantly different ways in which MK-2206 and MKHNE affect phosphorylation at T309 within Akt2 and T305 within Akt3. A similar experiment was carried out, but lysates were analyzed for pT309 of Akt2 and T305 of Akt3WT and Akt3C119S mutant using ratiometric sandwich ELISA. FIG. 23F is a graph showing proliferation curves. The stated lines were plated in 96-well black-sided plates (3000 cells per well) and treated with varying concentrations of MK-FNE or MK-2206 for 3 days (T47D MK-2206-res: T47D MK-2206-resistant lines). After this time, the number of viable cells was calculated using AlamarBlue®. Proliferation curves were fitted to the PI₅₀ equation (EQN. 4). FIG. 23G is a graph showing cell lines that are more than 2-fold more sensitive to MK-2206 than MK-FNE. To derive 23G, data from FIG. 23F was reanalyzed by dividing PI₅₀(MK-FNE) by PI₅₀(MK-2206) to give selectivity. FIG. 23H is a graph showing the percentage of viable cells. MDA-MB-468 were treated with the compound at the EC₆₀ concentration for 48 hours (i.e. MK-2206 (2 μM), MK-FNE (3 μM), and MK-HNE (6 μM)), then the compound was removed and cells allowed to recover for a further 24 hours. The percentage of growth relative to DMSO-treated cells is shown for the 48 hour point and the 72 hour point. FIG. 23I is a schematic showing the different methods used to derive the data in 23H.

FIG. 24 is a schematic showing a proposed mechanism for drug synergism with Akt isoform-specific knockdown.

FIGS. 25A-E show representative flow cytometry data.

### DETAILED DESCRIPTION OF THE INVENTION

One aspect of the present invention relates to a compound of Formula (I):

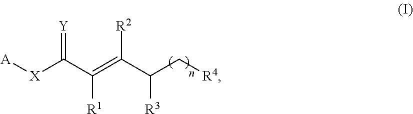

wherein

A is a reversible pan-Akt inhibitor or a radical thereof;

X is optional, and, if present, is NH, O, or S;

Y is O or S;

R¹ is H, halogen, NO₂, CN, C₁₋₆ alkyl, or aryl;

R² is H, halogen, NO₂, CN, C₁₋₆ alkyl, or aryl;

R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;

R⁵ is H, C₁₋₆ alkyl, or aryl;

R⁶ is H, C₁₋₆ alkyl, or aryl; and

n is 0, 1, 2, 3, 4, or 5;

or an oxide thereof, a pharmaceutically acceptable salt thereof, a solvate thereof, or a prodrug thereof.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “alkenyl” means an aliphatic hydrocarbon group containing a carbon-carbon double bond and which may be straight or branched having about 2 to about 6 carbon atoms in the chain. Particular alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. The term “alkenyl” may also refer to a hydrocarbon chain having 2 to 6 carbons containing at least one double bond and at least one triple bond.

The term “alkynyl” means an aliphatic hydrocarbon group containing a carbon-carbon triple bond and which may be straight or branched having about 2 to about 6 carbon atoms in the chain. Particular alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl.

The term “cycloalkyl” means a non-aromatic mono- or multicyclic ring system of about 3 to about 7 carbon atoms, preferably of about 5 to about 7 carbon atoms. Exemplary monocyclic cycloalkyls include cyclopentyl, cyclohexyl, cycloheptyl, and the like.

The term “aryl” means an aromatic monocyclic or multicyclic ring system of 6 to about 14 carbon atoms, preferably of 6 to about 10 carbon atoms. Representative aryl groups include phenyl and naphthyl.

The term “monocyclic” used herein indicates a molecular structure having one ring.

The term “multicyclic” used herein indicates a molecular structure having two or more rings, including, but not limited to, fused, bridged, or spiro rings.

The term “alkoxy” means groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy, and the like. Lower-alkoxy refers to groups containing one to four carbons. For the purposes of the present patent application, alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

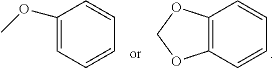

The term “halo” or “halogen” means fluoro, chloro, bromo, or iodo.

The term “substituted” or “substitution” of an atom means that one or more hydrogen on the designated atom is replaced with a selection from the indicated group, provided that the designated atom's normal valency is not exceeded.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. “Unsubstituted” atoms bear all of the hydrogen atoms dictated by their valency. When a substituent is keto (i.e., ═O), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “method of treating” means amelioration or relief from the symptoms and/or effects associated with the disorders described herein. As used herein, reference to “treatment” of a patient is intended to include prophylaxis.

The term “compounds of the invention”, and equivalent expressions, are meant to embrace compounds of general Formula (I), Formula (Ia), Formula (Ib), and Formula (Ic) as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The term “pharmaceutically acceptable salts” means the relatively non-toxic, inorganic, and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like (see, for example, Berge et al., “Pharmaceutical Salts,” J. Pharm. Sci., 66:1-9 (1977) and Remington's Pharmaceutical Sciences, 17th ed., Mack Publishing Company, Easton, Pa., 1985, p. 1418, which are hereby incorporated by reference in their entirety). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include, for example, sodium hydride, sodium hydroxide, potassium hydroxide, calcium hydroxide, aluminium hydroxide, lithium hydroxide, magnesium hydroxide, and zinc hydroxide. Suitable amine base addition salts are prepared from amines which have sufficient basicity to form a stable salt, and preferably include those amines which are frequently used in medicinal chemistry because of their low toxicity and acceptability for medical use, such as ammonia, ethylenediamine, N-methyl-glucamine, lysine, arginine, omithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, dicyclohexylamine, and the like.

The term “pharmaceutically acceptable prodrugs” as used herein means those prodrugs of the compounds useful according to the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. The term “prodrug” means compounds that are rapidly transformed in vivo to yield the parent compound of the above formula, for example by hydrolysis in blood. Functional groups which may be rapidly transformed, by metabolic cleavage, in vivo form a class of groups reactive with the carboxyl group of the compounds of this invention. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier (1985); Methods in Enzymology, K. Widder et al, Ed., Academic Press, 42, p. 309-396 (1985); A Textbook of Drug Design and Development, Krogsgaard-Larsen and H. Bundgaard, ed., Chapter 5; “Design and Applications of Prodrugs” p. 113-191 (1991); Advanced Drug Delivery Reviews, H. Bundgard, 8, p. 1-38 (1992); J. Pharm. Sci., 77:285 (1988); Nakeya et al, Chem. Pharm. Bull., 32:692 (1984); Higuchi et al., “Pro-drugs as Novel Delivery Systems,” Vol. 14 of the A.C.S. Symposium Series, and Bioreversible Carriers in Drug Design, Edward B. Roche, ed., American Pharmaceutical Association and Pergamon Press (1987), which are incorporated herein by reference in their entirety. Examples of prodrugs include, but are not limited to, acetate, formate, and benzoate derivatives of alcohol and amine functional groups in the compounds of the invention.

The term “solvate” refers to a compound of Formula (I), Formula (Ia), Formula (Ib), and Formula (Ic) in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The term “therapeutically effective amounts” is meant to describe an amount of compound of the present invention effective to produce the desired therapeutic effect. Such amounts generally vary according to a number of factors well within the purview of ordinarily skilled artisans given the description provided herein to determine and account for. These include, without limitation: the particular subject, as well as its age, weight, height, general physical condition, and medical history; the particular compound used, as well as the carrier in which it is formulated and the route of administration selected for it; and, the nature and severity of the condition being treated.

The term “pharmaceutical composition” means a composition comprising a compound of Formula (I), Formula (Ia), Formula (Ib), and Formula (Ic) and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifingal agents, lubricating agents and dispensing agents, depending on the nature of the mode of administration and dosage forms. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgement, suitable for use in contact with the cells of humans and lower animals without undue toxicity, irritation, allergic response and the like, and are commensurate with a reasonable benefit/risk ratio.

The term “pharmaceutically acceptable dosage forms” means dosage forms of the compound of the invention, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques.

The compounds described herein contain olefinic double bonds and may also have other centers of geometric asymmetry, and unless specified otherwise, it is intended that the compounds include both E and Z geometric isomers. Likewise, all tautomeric forms are also intended to be included.

The compound of Formula (I) can be present as a cis isomer or a trans isomer. In one embodiment, the compound of Formula (I) is a cis isomer. In another embodiment, the compound of Formula (I) is a trans isomer. In yet another embodiment, the compound of Formula (I) is a mixture of isomers.

In one embodiment, compound of Formula (I) has the Formula (Ia):

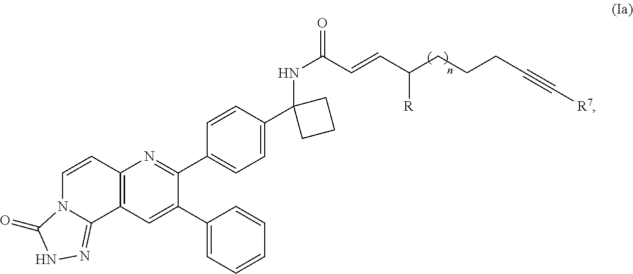

wherein

R is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁷ is H, CN, CF₃, CO₂H, SiMe₃, C₁₋₆ alkyl, or C₁₋₆ alkoxy; and

m is 0, 1, 2, or 3,

or an oxide thereof, a pharmaceutically acceptable salt thereof, a solvate thereof, or a prodrug thereof.

In another embodiment, compound of Formula (I) has the Formula (Ib):

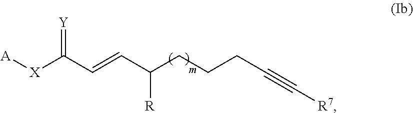

wherein

A is a reversible pan-Akt inhibitor or a radical thereof;

X is optional, and, if present, is NH, O, or S;

Y is O or S;

R is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁷ is H, CN, CF₃, CO₂H, SiMe₃, C₁₋₆ alkyl, or C₁₋₆ alkoxy; and

m is 0, 1, 2, or 3,

or an oxide thereof, a pharmaceutically acceptable salt thereof, a solvate thereof, or a prodrug thereof.

In another embodiment, compound of Formula (I) has the Formula (Ic):

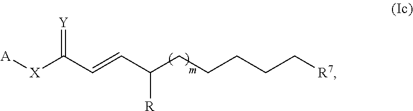

wherein

A is a reversible pan-Akt inhibitor or a radical thereof;

X is optional, and, if present, is NH, O, or S;

Y is O or S;

R is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁷ is H, CN, CF₃, CO₂H, SiMe₃, C₁₋₆ alkyl, or C₁₋₆ alkoxy; and

m is 0, 1, 2, or 3,

or an oxide thereof, a pharmaceutically acceptable salt thereof, a solvate thereof, or a prodrug thereof.

In one embodiment, X is absent. In an alternative embodiment, X is present and is NH, O, or S.

Any reversible pan-Akt inhibitor or a radical thereof can be used in accordance with the present invention. Suitable pan-Akt inhibitors include: MK-2206, Perifosine (KRX-0401), GSK690693, Ipatasertib (GDC-0068), AZD5363, PF-04691502, AT7867, Triciribine, CCT128930, A-674563, PHT-427, Miltefosine, Honokiol, TIC10 Analogue, Uprosertib (GSK2141795), TIC10, Akti-1/2, Miransertib (ARQ 092), Afuresertib (GSK2110183), AT13148, Deguelin, and SC79, and pharmaceutically acceptable salts thereof.

In one embodiment, the radical of a pan-Akt inhibitor has the Formula:

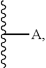

wherein A is the group obtained after the removal of one of the hydrogen atoms from the chemical structure of the pan-Akt inhibitor (A-H).

In one embodiment, A is a radical of a compound selected from the group consisting of MK-2206, GSK690693, inhibitor VIII, and Akt-I-1,2 (2-[4-(3-phenylquinoxalin-2-yl)phenyl]propan-2-amine) (CAS #473382-48-8).

In another embodiment, A is a radical of a compound selected from the group consisting of MK-2206, GSK690693, and Akt-I-1,2 (2-[4-(3-phenylquinoxalin-2-yl)phenyl]propan-2-amine) (CAS #473382-48-8).

In another embodiment, A is a radical of GSK690693 or inhibitor VIII.

In one embodiment, A is a radical of MK-2206 and has the following structure:

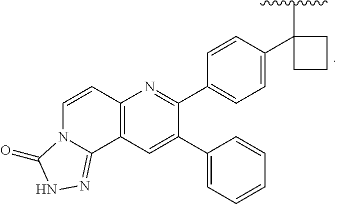

In another embodiment, A is a radical of GSK690693 and has one of the following structures:

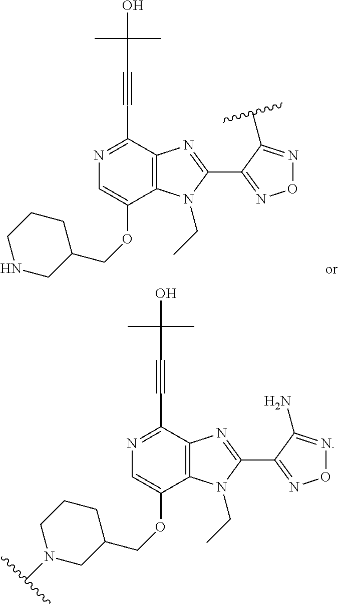

In another embodiment, A is a radical of inhibitor VIII and has the following structure:

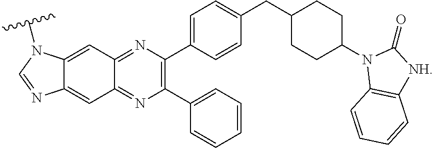

In another embodiment, A is a radical of Akt-I-1,2 (2-[4-(3-phenylquinoxalin-2-yl)phenyl]propan-2-amine) (CAS #473382-48-8) and has the following structure:

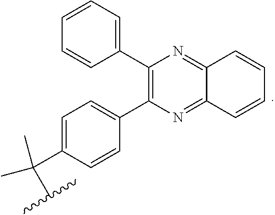

A further embodiment of the present invention relates to the compounds having one of the following structures:

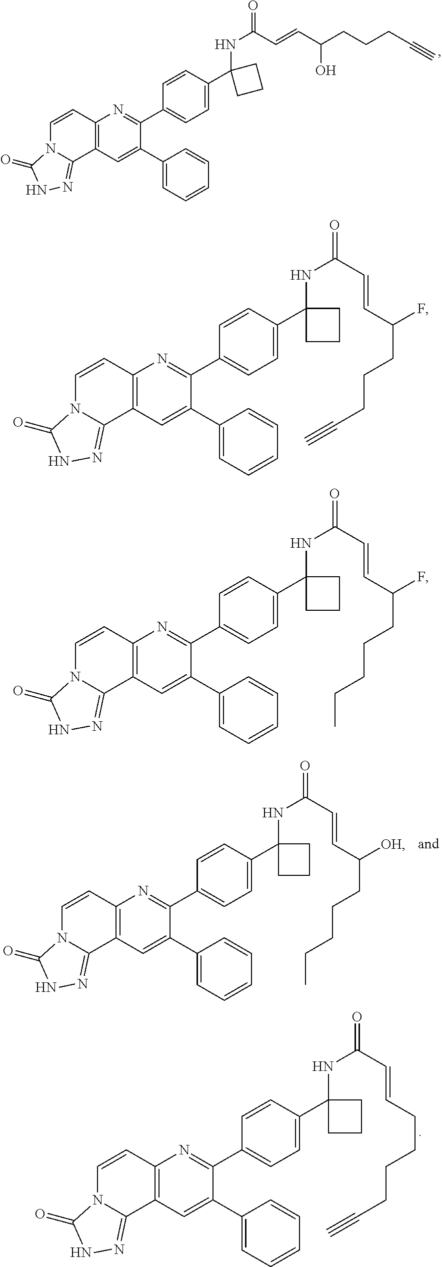 [SMILES: C#CCCCC(O)/C=C/C(=O)NC1(c2ccc(-c3nc4ccn5c(=O)[nH]nc5c4cc3-c3ccccc3)cc2)CCC1 ; C#CCCCC(F)/C=C/C(=O)NC1(c2ccc(-c3nc4ccn5c(=O)[nH]nc5c4cc3-c3ccccc3)cc2)CCC1 ; CCCCCC(F)/C=C/C(=O)NC1(c2ccc(-c3nc4ccn5c(=O)[nH]nc5c4cc3-c3ccccc3)cc2)CCC1 ; CCCCCC(O)/C=C/C(=O)NC1(c2ccc(-c3nc4ccn5c(=O)[nH]nc5c4cc3-c3ccccc3)cc2)CCC1 ; C#CCCCC/C=C/C(=O)NC1(c2ccc(-c3nc4ccn5c(=O)[nH]nc5c4cc3-c3ccccc3)cc2)CCC1]

Another embodiment of the present invention relates to compounds having the following structures:

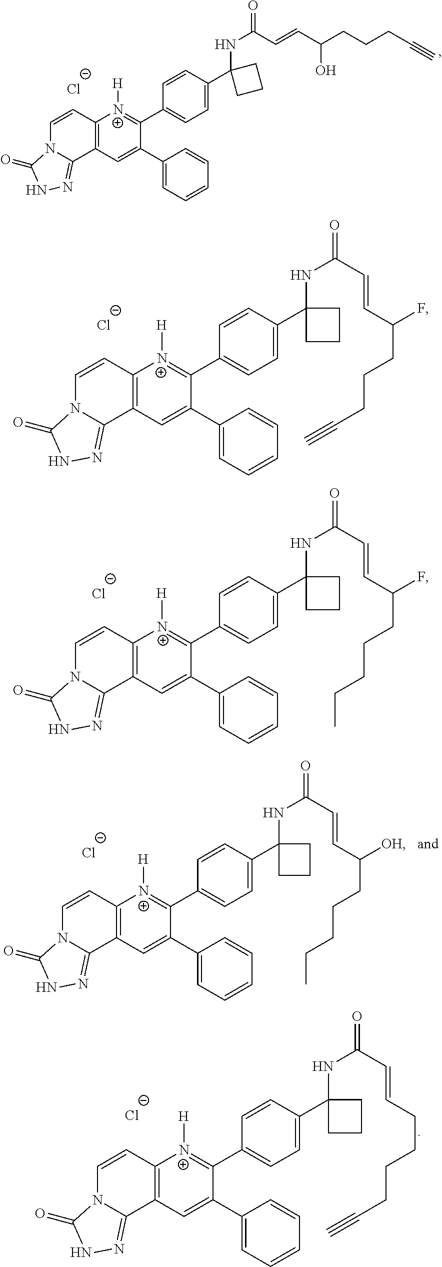

Compounds of the present invention can be produced according to Scheme 1 outlined below.

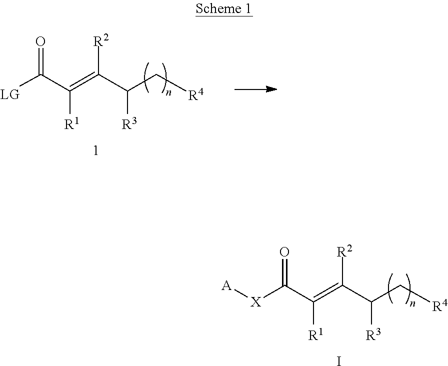

Reaction of the carboxylic acid derivative (1) with any reversible pan-Akt inhibitor, A-H, leads to formation of the compound of Formula (I). The reaction can be carried out in a variety of solvents, for example in methylene chloride (CH₂Cl₂), tetrahydrofiuran (THF), dimethylformamide (DMF), or other such solvents or in the mixture of such solvents During the reaction process, the non-participating carboxylic acids, alcohols, or amines on the molecule can be protected by a suitable protecting group which can be selectively removed at a later time if desired (Schemes 2 and 3). A detailed description of these groups and their selection and chemistry is contained in “The Peptides, Vol. 3”, Gross and Meinenhofer, Eds., Academic Press, New York, 1981, which is hereby incorporated by reference in its entirety. Thus, useful protective groups for the carboxylic acid group are methyl, t-butyl, benzyl, silyl, oxazoline, and the like. Useful protective groups for the alcohols are methoxymethyl (MOM), tetrahydropyranyl (THP), t-butyl, benzyl, t-butyldimethylsilyl (TBDMS), t-butyldiphenylsilyl (TBDPS), and the like. Useful protective groups for the amino group are benzyloxycarbonyl (Cbz), t-butyloxycarbonyl (t-BOC), 2,2,2-trichloroethoxycarbonyl (Troc), t-amyloxycarbonyl, 4-methoxybenzyloxycarbonyl, 2-(trichlorosilyl)ethoxycarbonyl, 9-fluorenylmethoxycarbonyl (Fmoc), phthaloyl, acetyl (Ac), formyl, trifluoroacetyl, and the like

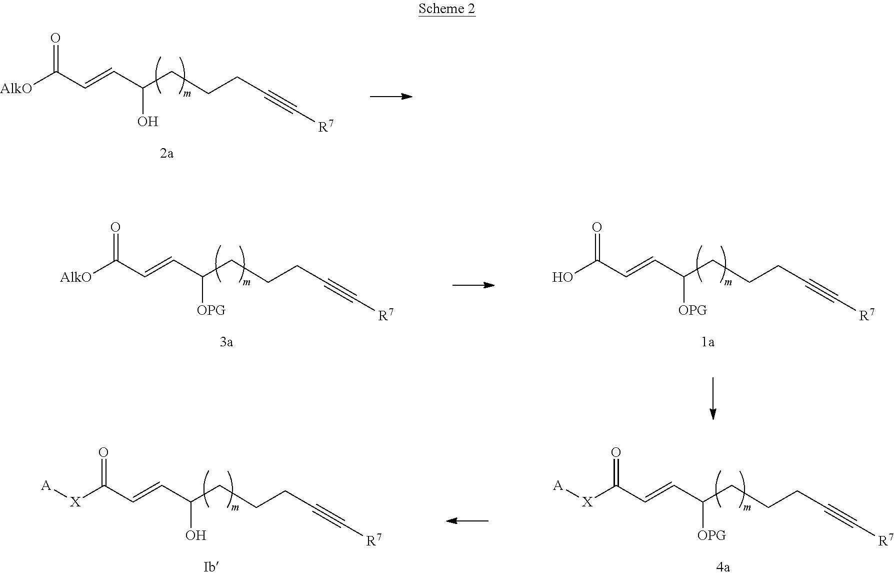

For example, as shown in Scheme 2, the hydroxyl group in the ester (2a) can be first protected with a suitable protecting group, and then the ester group is hydrolyzed, resulting in the compound of Formula (1a). Reaction of the carboxylic acid derivative (1a) with any reversible pan-Akt inhibitor, A-H, leads to formation of the compound (4a). The reaction can be carried out in a variety of solvents, for example in methylene chloride (CH₂Cl₂), tetrahydrofuran (THF), dimethylformamide (DMF), or other such solvents or in the mixture of such solvents. The compound of Formula (Ib′) can be obtained following the deprotection of the hydroxyl group in the compound (4a).

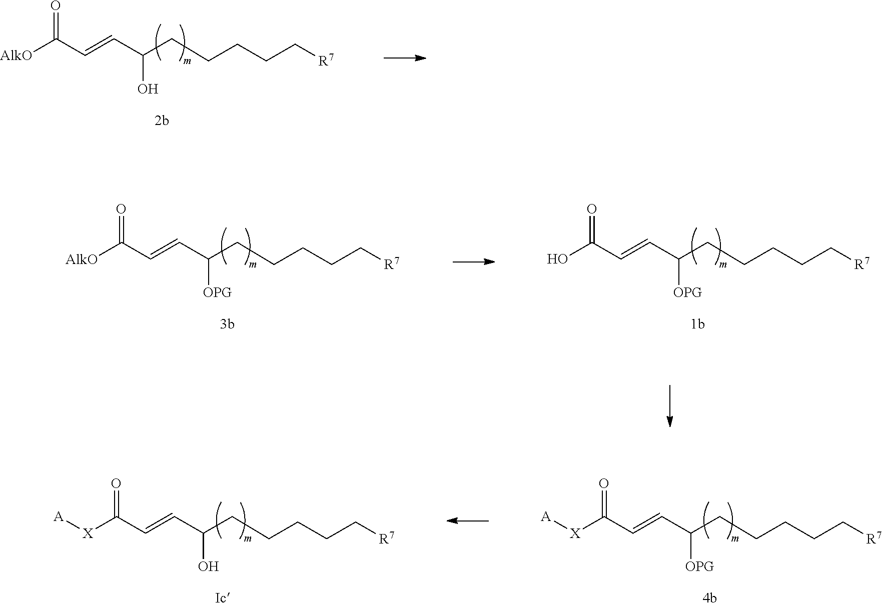

As shown in Scheme 3, the hydroxyl group in ester (2b) can be first protected with a suitable protecting group, and then the ester group is hydrolyzed resulting in compound (1b). Reaction of the carboxylic acid derivative (1b) with any reversible pan-Akt inhibitor, A-H, leads to formation of the compound of Formula (4b). The reaction can be carried out in a variety of solvents, for example in methylene chloride (CH₂Cl₂), tetrahydrofuran (THF), dimethylformamide (DMF), or other such solvents or in the mixture of such solvents. The compound of Formula (Ic′) can be obtained following the deprotection of the hydroxyl group the compound (4b).

The carboxylic acid derivative (1) can be prepared according to the general Scheme 4.

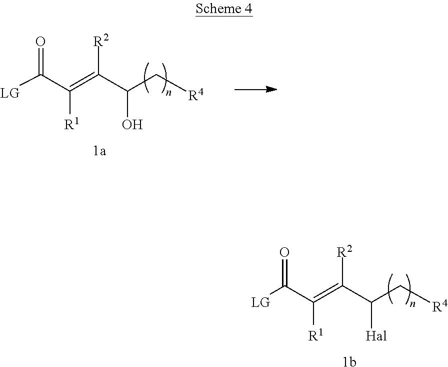

Reaction of the carboxylic acid derivative (1a) containing hydroxyl group with any suitable halogenating reagent leads to formation of the compound (1b). The reaction can be carried out in a variety of solvents, for example in methylene chloride (CH₂Cl₂), tetrahydrofuran (THF), dimethylformamide (DMF), or other such solvents or in the mixture of such solvents. Suitable halogenating reagents that can be used include DAST, SOCl₂, Et₂NCF₂CHClF, NCS, NBS, NaI, Ph₃P/Br₂, I₂, CCl₄, CBr₄.

The compounds of the present invention wherein Y is O can be easily converted to their analogs wherein Y is S, according to known methods. For example by using Lawesson's reagent.

While it may be possible for the compounds of the present invention to be administered as raw chemicals, it will often be preferable to present them as a part of a pharmaceutical composition. Accordingly, another aspect of the present invention is a pharmaceutical composition containing a therapeutically effective amount of the compound of the present invention, or a pharmaceutically acceptable salt or solvate thereof, and a pharmaceutically acceptable carrier. The carrier must be “acceptable” in the sense of being compatible with the other ingredients of the formulation and not deleterious to the recipient thereof.

Another aspect of the present invention relates to a method of treating cancer. This method includes administering to a subject a compound of the Formula (I):

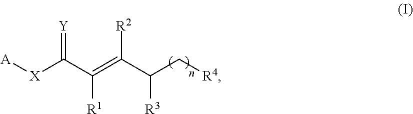

wherein

A is a reversible pan-Akt inhibitor or a radical thereof;

X is optional, and, if present, is NH, O, or S;

Y is O or S;

R¹ is H, halogen, NO₂, CN, C₁₋₆ alkyl, or aryl;

R² is H, halogen, NO₂, CN, C₁₋₆ alkyl, or aryl;

R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;

R⁵ is H, C₁₋₆ alkyl, or aryl;

R⁶ is H, C₁₋₆ alkyl, or aryl; and

n is 0, 1, 2, 3, 4, or 5;

or an oxide thereof, a pharmaceutically acceptable salt thereof, a solvate thereof, or a prodrug thereof,
under conditions effective to treat cancer in the subject.

In one embodiment, the method further includes selecting a subject having cancer mediated by Akt2 or Akt3, wherein the selected subject receives the administered compound. In one embodiment, cancer is treated. The cancer is selected from the group consisting of breast cancer, prostate cancer, colon cancer, lung cancer, and ovarian cancer.

Administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

Another aspect of the present invention relates to a method of inhibiting pan-Akt in a cell or a tissue. This method includes providing a compound of Formula (I):

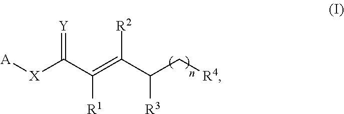

wherein

A is a reversible pan-Akt inhibitor or a radical thereof;

X is optional, and, if present, is NH, O, or S;

Y is O or S;

R¹ is H, halogen, NO₂, CN, C₁₋₆ alkyl, or aryl;

R² is H, halogen, NO₂, CN, C₁₋₆ alkyl, or aryl;

R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;

R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;

R⁵ is H, C₁₋₆ alkyl, or aryl;

R⁶ is H, C₁₋₆ alkyl, or aryl; and

n is 0, 1, 2, 3, 4, or 5;

or an oxide thereof, a pharmaceutically acceptable salt thereof, a solvate thereof, or a prodrug thereof; and
contacting a cell or tissue with the compound under conditions effective to inhibit pan-Akt.

In practicing the method of the present invention, agents suitable for treating a subject can be administered using any method standard in the art. The agents, in their appropriate delivery form, can be administered orally, intradermally, intramuscularly, intraperitoneally, intravenously, subcutaneously, or intranasally. The compositions of the present invention may be administered alone or with suitable pharmaceutical carriers, and can be in solid or liquid form, such as tablets, capsules, powders, solutions, suspensions, or emulsions.

The agents of the present invention may be orally administered, for example, with an inert diluent, or with an assimilable edible carrier, or it may be enclosed in hard or soft shell capsules, or it may be compressed into tablets, or they may be incorporated directly with the food of the diet. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

Also specifically contemplated are oral dosage forms of the agents of the present invention. The agents may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. (Abuchowski and Davis, “Soluble Polymer-Enzyme Adducts,” In: Enzymes as Drugs, Hocenberg and Roberts, eds., Wiley-Interscience, New York, N.Y., pp. 367-383 (1981), which are hereby incorporated by reference in their entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucrulose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agents of the present invention may also be administered parenterally. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The agents of the present invention may also be administered directly to the airways in the form of an aerosol. For use as aerosols, the agent of the present invention in solution or suspension may be packaged in a pressurized aerosol container together with suitable propellants, for example, hydrocarbon propellants like propane, butane, or isobutane with conventional adjuvants. The agent of the present invention also may be administered in a non-pressurized form such as in a nebulizer or atomizer.

Effective doses of the compositions of the present invention, for the treatment of cancer vary depending upon many different factors, including type and stage of cancer, means of administration, target site, physiological state of the patient, other medications or therapies administered, and physical state of the patient relative to other medical complications. Treatment dosages need to be titrated to optimize safety and efficacy.

The percentage of active ingredient in the compositions of the present invention may be varied, it being necessary that it should constitute a proportion such that a suitable dosage shall be obtained. Obviously, several unit dosage forms may be administered at about the same time. The dose employed will be determined by the physician, and depends upon the desired therapeutic effect, the route of administration and the duration of the treatment, and the condition of the patient. In the adult, the doses are generally from about 0.01 to about 100 mg/kg body weight, preferably about 0.01 to about 10 mg/kg body weight per day by inhalation, from about 0.01 to about 100 mg/kg body weight, preferably 0.1 to 70 mg/kg body weight, more especially 0.1 to 10 mg/kg body weight per day by oral administration, and from about 0.01 to about 50 mg/kg body weight, preferably 0.01 to 10 mg/kg body weight per day by intravenous administration. In each particular case, the doses will be determined in accordance with the factors distinctive to the subject to be treated, such as age, weight, general state of health, and other characteristics which can influence the efficacy of the medicinal product.

The products according to the present invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. It goes without saying that, for other patients, it will be necessary to prescribe not more than one or two doses per day.

Another aspect of the present invention relates to a compound of Formula (II):

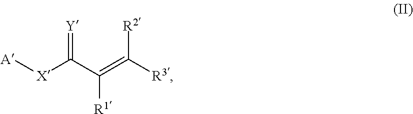

wherein

A′ is a reversible pan-Akt inhibitor or a radical thereof;

X′ is optional, and, if present, is NH, O, or S;

Y′ is O or S;

R^{1′} is H, halogen, NO₂, CN, C₁₋₆ alkyl, or aryl;

R^{2′} is H, halogen, NO₂, CN, C₁₋₆ alkyl, or aryl;

R^{3′} is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H, wherein C₁₋₆ alkyl and C₃₋₆ cycloalkyl can be optionally substituted from 1 to 3 times with a substituent selected independently at each occurrence thereof from the group consisting of H, halogen, C₁₋₆ alkyl, C₃₋₆ cycloalkyl, OC₁₋₆ alkyl, OH, NO₂, N₃, NR⁴′R⁵′, CN, CF₃, and CO₂H;

R^{4′} is H, C₁₋₆ alkyl, or aryl; and

R^{5′} is H, C₁₋₆ alkyl, or aryl,

or an oxide thereof, a pharmaceutically acceptable salt thereof, a solvate thereof, or a prodrug thereof.

The compounds of Formula (II) can be made and used as described above for the compounds of Formula (I).

### EXAMPLES

The examples below are intended to exemplify the practice of embodiments of the disclosure but are by no means intended to limit the scope thereof.

### Example 1—Materials and Methods

Unless otherwise stated, reactions were carried out under N₂ or Ar. Standard syringe and cannulating techniques were applied for transferring reagents to the reaction flask. Reaction progress was monitored with glass backed thin-layer chromatography (TLC) plates pre-coated with silica UV254 and visualized by UV at λ=254 or 365 nm, vanillin, 5% H₂SO₄, or potassium permanganate dip. Silica gel 60 (particle size 0.040-0.065 mm) was used in flash chromatography with the ratio of solvents indicated in the experimental section. All solvents used were either analytical or HPLC grade. MK-2206 for chemical synthesis was from eNovation Chemicals. ¹H and ¹³C NMR spectra were recorded on a Bruker Avance 500 (500 MHz for ¹H and 125 MHz for ¹³C) spectrometer and referenced to the residual solvent peaks of deuterated solvents. The data is reported as chemical shift (δ), multiplicity (app=apparent, s=singlet, d=doublet, t=triplet, q=quartet, dd=doublet of doublet, ddt=doublet of doublet of triplet, td=triplet of doublet, m=multiplet), coupling constant (J Hz) and relative integral.

All primers were from IDT. Phusion HotStart II polymerase was from Thermo Scientific. All restriction enzymes were from NEB. All Halo clones were contained in a pFN21a vector (Kazusa Collection) or the PCS2+8 vector (both of which drive expression from a CMV promoter). Complete EDTA free protease inhibitor was from Roche. 1×Bradford dye was from Bio-Rad. TALON metal affinity resin and His60 nickel resin were from Clonetech. Cy5 azide and Cu(TBTA) were from Lumiprobe. Dithiothreitol (DTT) and TCEP-HCl were from Goldbio Biotechnology. ATP disodium salt hydrate (ATP) was from Fisher. MK-2206.2HCl (MK-2206. S1078), afuresertib (S7521), GDC-0068 (S2808), and GSK-690693 (S1113) were from Selleckchem. 10 mM Stocks of the inhibitors were prepared in DMSO and stored in aliquots at −80° C. Streptavidin Sepharose beads were from GE Healthcare. BSA powder was from Thermo Scientific. All other chemicals and enzymes were from Sigma-Aldrich.

Akt3 kinase was obtained from Active Motif, cat. No. 31147. HEK293T cells were from American Type Culture Collection (ATCC). MDA-MB-468 and BT-549 Cell lines were gifts from the Cantley lab, Weill Cornell Medicine. MDA-MB-231, Hs578T, TSE, and 3T3 cell lines were gifts from the Cerione lab, College of Veterinary Medicine, Cornell University Medical Center. T47D and T47D MK-2206-resistant lines were gifts from the Toker lab, Harvard Medical School. Cell lines were validated to be free of mycoplasma contamination using Venor™GeM Mycoplasma Detection Kit (MP0025, Sigma-Aldrich) every three months. All cell lines were used below passage 10. 1×DPBS, 1×Trypsin (TrypLe), 100×NEAA, 100× sodium pyruvale, 100 penicillin-streptomycin, and 1×MEM+Glutamax were from Life Technologies. FBS was from Sigma-Aldrich (F2442). TransIT 2020 transfection reagent was from Mirus Bio LLC. All tissue-culture treated plasticware was from CellTreat. For all confocal imaging experiments, a Zeiss LSM710 confocal microscope was used. Imaging plates (35 mm glass bottom dish) were from Cellvis (D35-20-0-N). Quantification of fluorescence intensity was performed using ImageJ software (NIH, version 1.50 g). In-gel fluorescence analysis and imaging of western blots and Coomassie-stained gels were performed using Bio-Rad Chemi-Doc MP Imaging system. Densitometric quantitation was performed using ImageJ (NIH). Cy5 excitation source was detected using epi-illumination and 695+/−28 nm emission filter was used. Cell counting was done by Countess II FL (A25750).

### Example 2—General Procedure for Coupling Lipid Acid to MK-2206

To a solution of acid (0.06 mmol, 2.0 eq.) in DMF (1.0 mL) at 0° C. was added EDC (14 mg, 0.07 mmol, 2.5 eq.) and iPrEt₂N (23 μL, 0.13 mmol, 17 mg, 4.0 eq.). After 5 min, a solution of MK-2206 (15 mg, 0.03 mmol, 1.0 eq.) in DMF (1.0 mL) with iPrEt₂N (11 μL, 0.07 mmol, 9 mg, 4.0 eq.) was added dropwise. The resulting reaction mixture was warmed to room temperature and stirred overnight. The crude reaction mixture was poured into water (20 mL) and extracted with EtOAc (3×10 mL). The organic fractions were combined and concentrated in vacuo.

### Example 3—General Procedure for Acid Deprotection of t-Butyldimethylsilyl (TBDMS) Ethers

The TBDMS ether (0.014 mmol) was added to TFA/MeOH (9:1 v/v, 10 mL) and stirred for 4 hours. The solvent was evaporated under a stream of compressed gas overnight. The crude mixture was re-suspended in 0.1 M NaOH solution (20 mL) and extracted with EtOAc (3×20 mL). The organic fraction was dried over Na₂SO₄, filtered, and evaporated in vacuo.

### Example 4—Synthesis of Synthesis of (E)-8-(4-(1-(4-Hydroxynon-2-en-8-ynamido)cyclobutyl)phenyl)-3-oxo-9-phenyl-2,3-dihydro-[1,2,4]triazolo[3,4-f][1,6]naphthyridin-7-ium Chloride (MK-HNE)

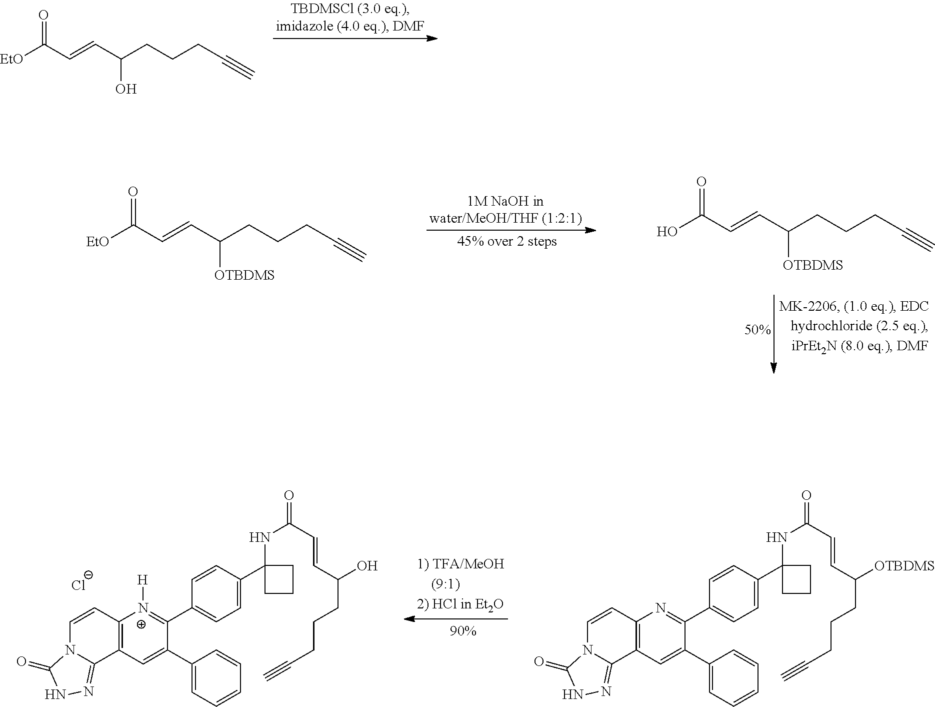 [SMILES: C#CCCCC(O)/C=C/C(=O)OCC ; C#CCCCC(/C=C/C(=O)OCC)O[Si](C)(C)C(C)(C)C ; C#CCCCC(/C=C/C(=O)O)O[Si](C)(C)C(C)(C)C ; C#CCCCC(O)/C=C/C(=O)NC1(c2ccc(-c3[nH+]c4ccn5c(=O)[nH]nc5c4cc3-c3ccccc3)cc2)CCC1 ; [Cl-] ; C#CCCCC(/C=C/C(=O)NC1(c2ccc(-c3nc4ccn5c(=O)[nH]nc5c4cc3-c3ccccc3)cc2)CCC1)O[Si](C)(C)C(C)(C)C]

### Synthesis of Ethyl (E)-4-((tert-Butyldimethylsilyl)oxy)non-2-en-8-ynoate

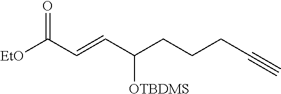 [SMILES: C#CCCCC(/C=C/C(=O)OCC)O[Si](C)(C)C(C)(C)C]

To a solution of alcohol (ethyl (E)-4-hydroxynon-2-en-8-ynoate) (200 mg, 1.0 mmol, 1.0 eq.) in DMF (5 mL) in ice was added imidazole (270 mg, 4.0 mmol, 4.0 eq.) and t-butyldimethylsilyl chloride (450 mg, 3.0 mmol, 3.0 eq.) and the resulting solution was allowed to stir overnight at room temperature. The crude reaction mixture was poured into 0.1 M HCl solution (50 mL) and extracted with Et₂O (3×10 mL). The combined organic extracts were dried over Na₂SO₄ and concentrated in vacuo. The crude product was further used without purification.

### Synthesis of (E)-4-((tert-Butyldimethylsilyl)oxy)non-2-en-8-ynoic Acid

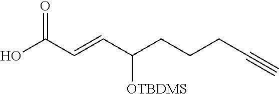 [SMILES: C#CCCCC(/C=C/C(=O)O)O[Si](C)(C)C(C)(C)C]

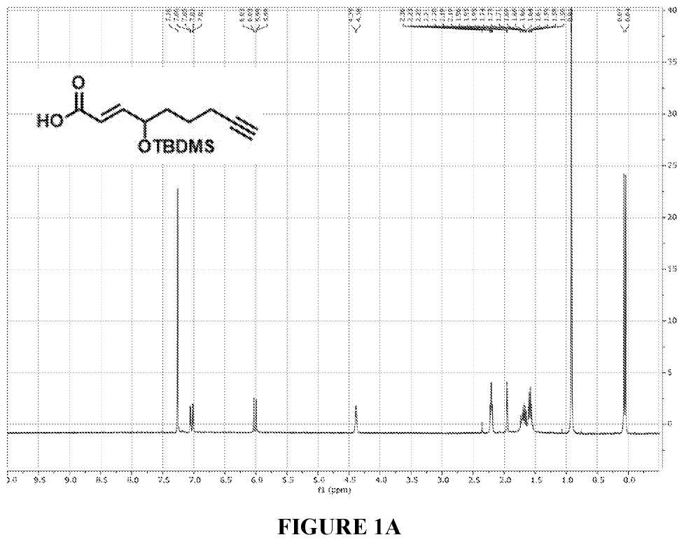

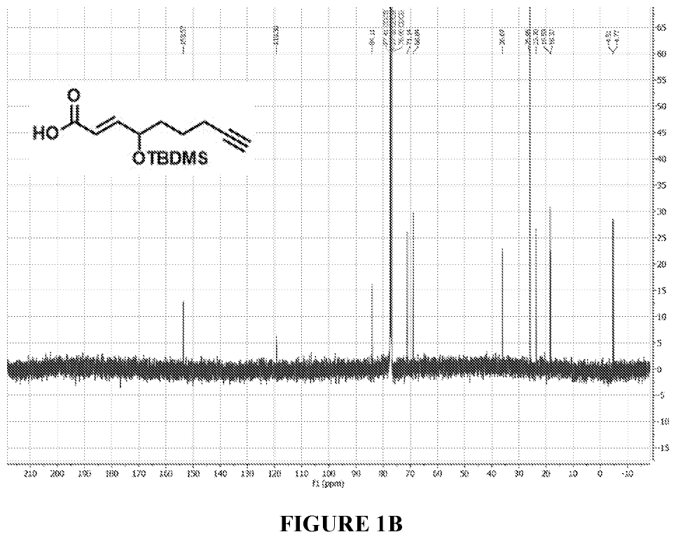

To a solution of crude ester (ethyl (E)-4-((tert-butyldimethylsilyl)oxy)non-2-en-8-ynoate) (205 mg, 0.65 mmol, 1.0 eq.) in MeOH/THF (2:1 v/v, 30 mL) was added 4 M NaOH solution (10 mL) dropwise. The reaction mixture was stirred at room temperature until TLC analysis indicated reaction completion. 3 M HCl solution was added dropwise until pH reach 2.0. This was followed by extraction with Et₂O (3×20 mL). The organic fractions were combined and concentrated in vacuo. The crude product was purified by flash chromatography (from 10:1 v/v, hexane:Et₂O with 1% AcOH) to afford the desired product as a yellow oil (130 mg, 45% over 2 steps). ¹H NMR (400 MHz, CDCl₃) δ 7.03 (dd, J=15.5, 4.4 Hz, 1H), 6.01 (dd, J=15.5, 1.5 Hz, 1H), 4.39 (app. q, J=4.9 Hz, 1H), 2.22 (dd, J=6.9, 2.7 Hz, 2H), 1.74-1.64 (m, 1H), 1.62-1.54 (m, 4H), 0.91 (s, 9H), 0.07 (s, 3H), 0.04 (s, 3H) (FIG. 1A). ¹³C NMR (125 MHz, CDCl₃) δ 153.4, 119.3, 84.1, 71.1, 68.8, 36.1, 25.9, 23.7, 18.5, 18.3, −4.5, −4.8 (FIG. 1B).

### Synthesis of (E)-4-((tert-Butyldimethylsilyl)oxy)-N-(1-(4-(3-oxo-9-phenyl-2,3-dihydro-[1,2,4]triazolo[3,4-f][1,6]naphthyridin-8-yl)phenyl)cyclobutyl)non-2-en-8-ynamide

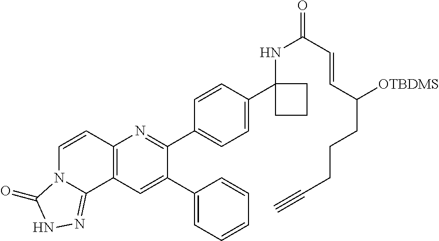 [SMILES: C#CCCCC(/C=C/C(=O)NC1(c2ccc(-c3nc4ccn5c(=O)[nH]nc5c4cc3-c3ccccc3)cc2)CCC1)O[Si](C)(C)C(C)(C)C]

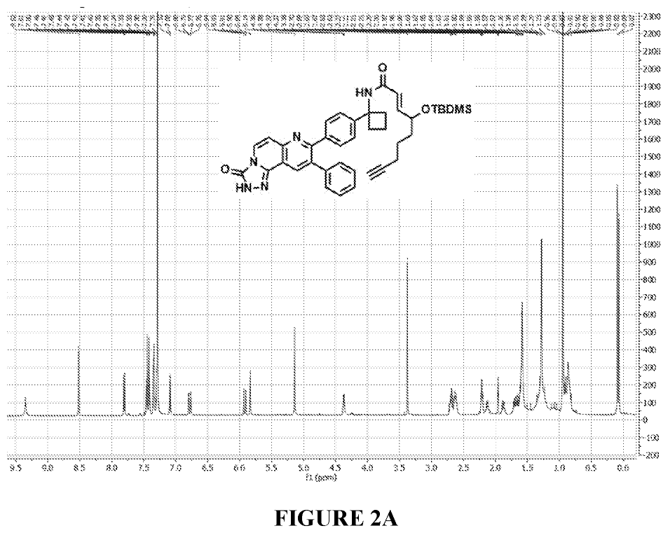

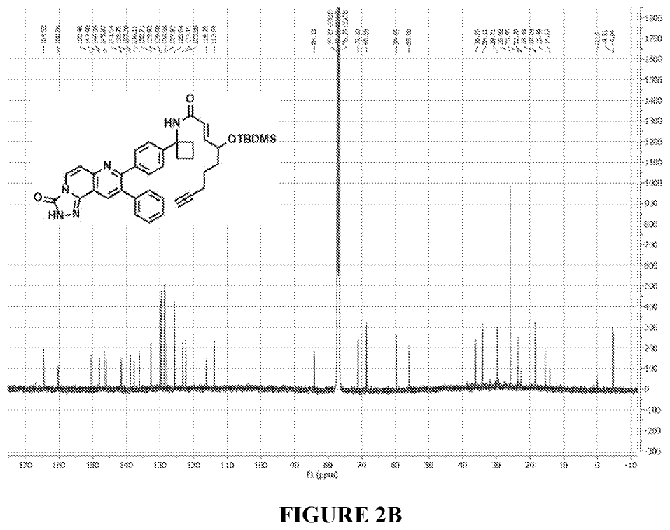

The coupling reaction followed general procedure described in Example 2. The crude product was purified by flash chromatography (from 4:1 v/v, toluene:Et₂OAc) to afford the desired product as a white solid (10 mg, 50%). ¹H NMR (500 MHz, CDCl₃) δ 9.35 (s, 1H), 8.52 (s, 1H), 7.81 (d, J=7.6 Hz, 1H), 7.45 (d, J=8.5 Hz, 2H), 7.41 (d, J=8.4 Hz, 2H), 7.35-7.33 (m, 3H), 7.30 (m, 2H), 7.09 (d, J=7.6 Hz, 1H), 6.78 (dd, J=15.1, 4.3 Hz, 1H), 5.92 (dd, J=15.1 Hz, 1H), 5.14 (s, 1H), 4.37 (m, 1H), 2.74-2.58 (m, 4H), 2.21 (td, J=7.2, 2.6 Hz, 2H), 2.12 (m, 1H), 1.96 (t, J=2.6 Hz, 1H), 1.88 (dt, J=8.4, 4.8 Hz, 1H), 1.74-1.62 (m, 1H), 1.64-1.55 (m, 4H), 0.94 (s, 9H), 0.09 (s, 3H), 0.07 (s, 3H) (FIG. 2A). ¹³C NMR (125 MHz, CD₃OD) δ164.52, 160.26, 150.46, 147.98, 146.58, 145.92, 141.54, 138.75, 137.70, 136.11, 132.71, 129.93, 129.58, 128.58, 127.92, 125.64, 123.15, 122.35, 116.25, 113.84, 84.13, 71.10, 68.59, 59.65, 55.99, 36.26, 34.11, 29.71, 25.92, 23.55, 18.43, 18.24, 15.49, −4.51, −4.84 (FIG. 2B).

### Synthesis of MK-HNE

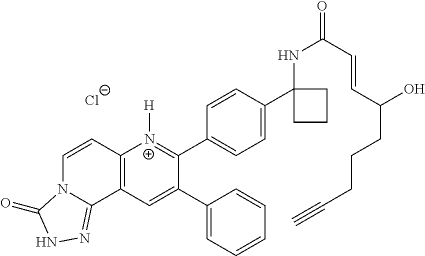 [SMILES: C#CCCCC(O)/C=C/C(=O)NC1(c2ccc(-c3[nH+]c4ccn5c(=O)[nH]nc5c4cc3-c3ccccc3)cc2)CCC1 ; [Cl-]]

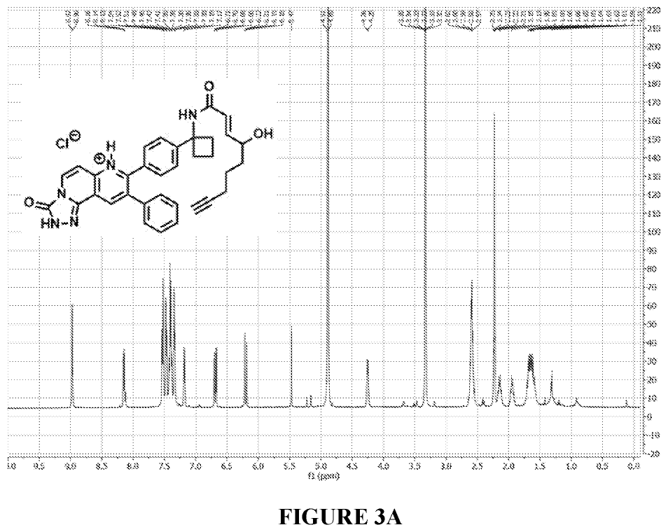

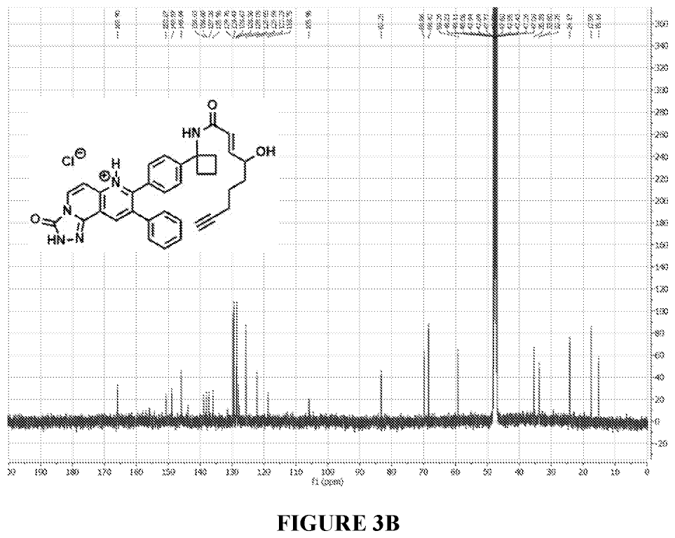

The silyl ether deprotection reaction followed the general procedure described in Example 3. The resulting crude product was re-dissolved in CH₂Cl₂ and precipitated by slow addition of 1 M HCl in Et₂O to afford the desired product as yellow powder (7.5 mg, 90%). ¹H NMR (500 MHz, CD₃OD) δ 8.97 (s, 1H), 8.15 (d, J=7.5 Hz, 1H), 7.53 (d, J=7.7 Hz, 2H), 7.47 (d, J=7.9 Hz, 2H), 7.42-7.38 (m, 3H), 7.40-7.31 (m, 2H), 7.18 (d, J=7.5 Hz, 1H), 6.69 (dd, J=15.4, 5.1 Hz, 1H), 6.20 (dd, J=15.4 Hz, 1H), 4.25 (m, 1H), 2.63-2.55 (m, 4H), 2.26-2.20 (m, 3H), 2.17-2.12 (m, 1H), 1.99-1.92 (m, 1H), 1.71-1.57 (m, 4H) (FIG. 3A). ¹³C NMR (125 MHz, CD₃OD) δ 165.90, 150.67, 148.87, 145.94, 138.93, 138.00, 137.28, 135.96, 129.76, 129.47, 128.67, 128.56, 128.09, 125.65, 125.59, 122.23, 118.75, 105.96, 83.25, 69.86, 68.42, 59.20, 35.39, 33.80, 33.75, 24.17, 17.50, 15.16 (FIG. 3B).

### Example 5—Synthesis of Synthesis of (E)-8-(4-(1-(4-Hydroxynon-2-enamido)cyclobutyl)phenyl)-3-oxo-9-phenyl-2,3-dihydro-[1,2,4]triazolo[3,4-f][1,6]naphthyridin-7-ium Chloride (MK-HNE No Alkyne)

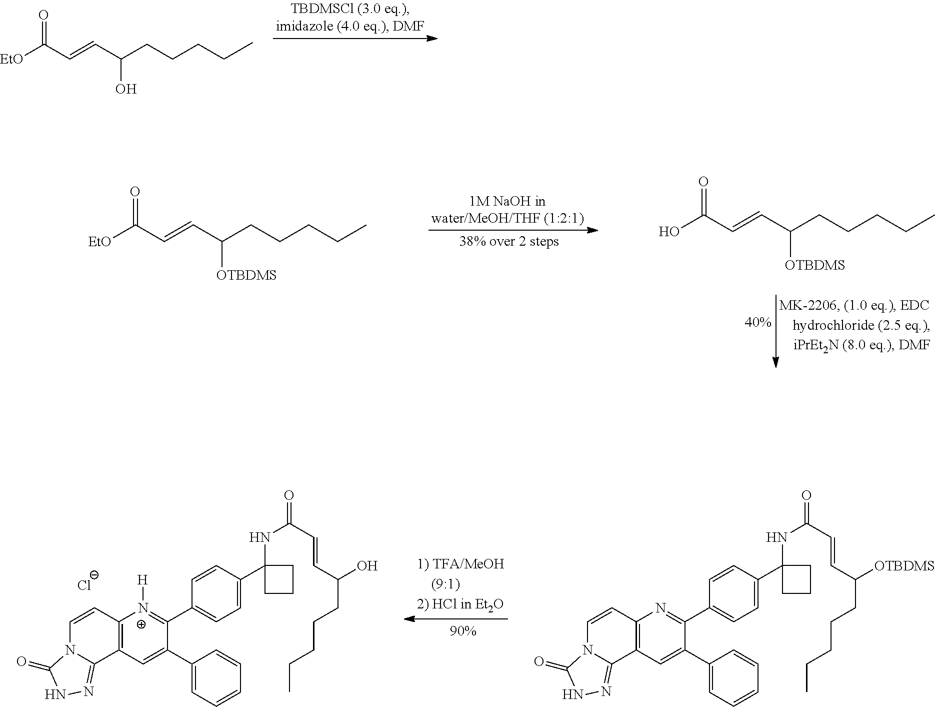 [SMILES: CCCCCC(O)/C=C/C(=O)OCC ; CCCCCC(/C=C/C(=O)OCC)O[Si](C)(C)C(C)(C)C ; CCCCCC(/C=C/C(=O)O)O[Si](C)(C)C(C)(C)C ; CCCCCC(O)/C=C/C(=O)NC1(c2ccc(-c3[nH+]c4ccn5c(=O)[nH]nc5c4cc3-c3ccccc3)cc2)CCC1 ; [Cl-] ; CCCCCC(/C=C/C(=O)NC1(c2ccc(-c3nc4ccn5c(=O)[nH]nc5c4cc3-c3ccccc3)cc2)CCC1)O[Si](C)(C)C(C)(C)C]

### Synthesis of Ethyl (E)-4-((tert-Butyldimethylsilyl)oxy)non-2-enoate

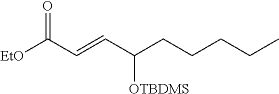 [SMILES: CCCCCC(/C=C/C(=O)OCC)O[Si](C)(C)C(C)(C)C]

To a solution of alcohol (ethyl (E)-4-hydroxynon-2-enoate) (1.8 g, 9.0 mmol, 1.0 eq.) in DMF (30 mL) in ice was added imidazole (1.6 g, 23 mmol, 2.6 eq.) and t-butyldimethylsilyl chloride (2.7 g, 18 mmol, 2.0 eq.) and the resulting solution was allowed to stir overnight at room temperature. The crude reaction mixture was poured into 0.1 M HCl solution (200 mL) and extracted with Et₂O (3×50 mL). The combined organic extracts were dried over Na₂SO₄, and concentrated in vacuo. The crude product was further used without purification.

### Synthesis of (E)-4-((tert-Butyldimethylsilyl)oxy)non-2-enoic Acid

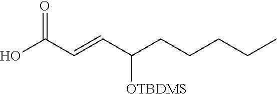 [SMILES: CCCCCC(/C=C/C(=O)O)O[Si](C)(C)C(C)(C)C]

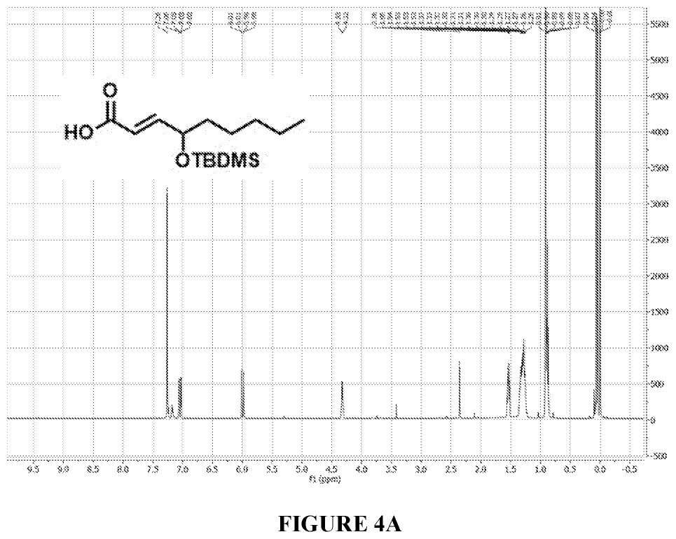

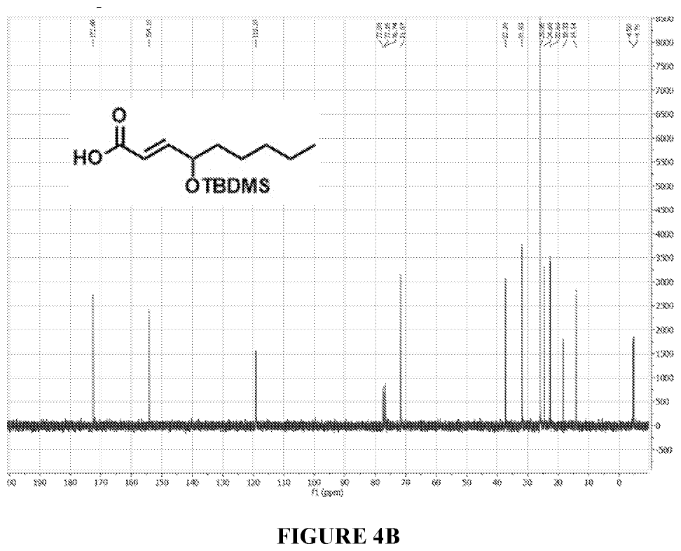

To a solution of ester (ethyl (E)-4-((tert-butyldimethylsilyl)oxy)non-2-enoate) (9.0 mmol, 1.0 eq.) in MeOH/THF (2:1 v/v, 120 mL) was added 4 M NaOH solution (40 mL) dropwise. The reaction mixture was stirred at room temperature until TLC analysis showed reaction completion. 3 M HCl solution was added dropwise until pH reach 2.0. This is followed by extraction with Et₂O (3×20 mL). The organic fractions were combined and concentrated in vacuo. The crude product was purified by flash chromatography (from 10:1 v/v, hexane:Et₂O with 1% AcOH) to afford the desired product as a colorless oil (900 mg, 38% over 2 steps). ¹H NMR (500 MHz, CDCl₃) δ 7.04 (dd, J=15.5, 4.5 Hz, 1H), 5.99 (dd, J=15.5, 1.7 Hz, 1H), 4.32 (m, 1H), 1.56-1.52 (m, 2H), 1.36-1.22 (m, 6H), 0.91 (s, 9H), 0.88 (t, J=5 Hz, 3H), 0.06 (s, 3H), 0.04 (s, 3H) (FIG. 4A). ¹³C NMR (75 MHz, CDCl₃) δ 172.60, 154.18, 119.18, 71.67, 37.30, 31.93, 25.95, 24.60, 22.69, 18.33, 14.14, −4.50, −4.78 (FIG. 4B).

### Synthesis of (E)-4-((tert-Butyldimethylsilyl)oxy)-N-(1-(4-(3-oxo-9-phenyl-2,3-dihydro-[1,2,4]triazolo[3,4-f][1,6]naphthyridin-8-yl)phenyl)cyclobutyl)non-2-enamide

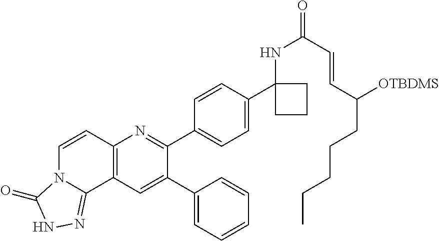 [SMILES: CCCCCC(/C=C/C(=O)NC1(c2ccc(-c3nc4ccn5c(=O)[nH]nc5c4cc3-c3ccccc3)cc2)CCC1)O[Si](C)(C)C(C)(C)C]

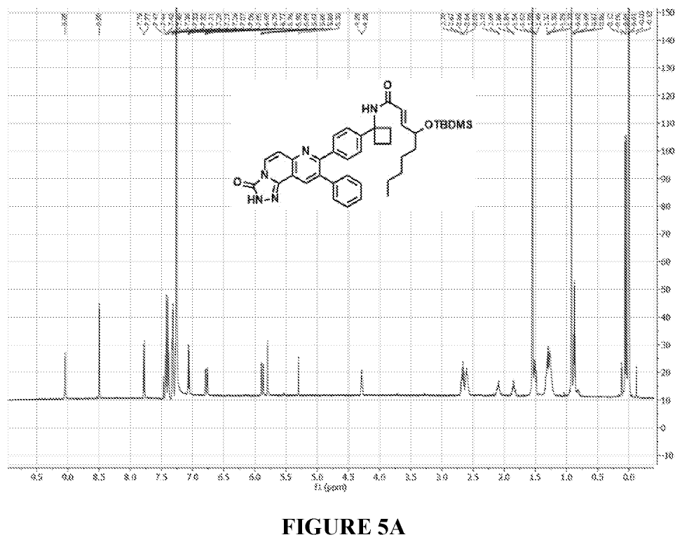

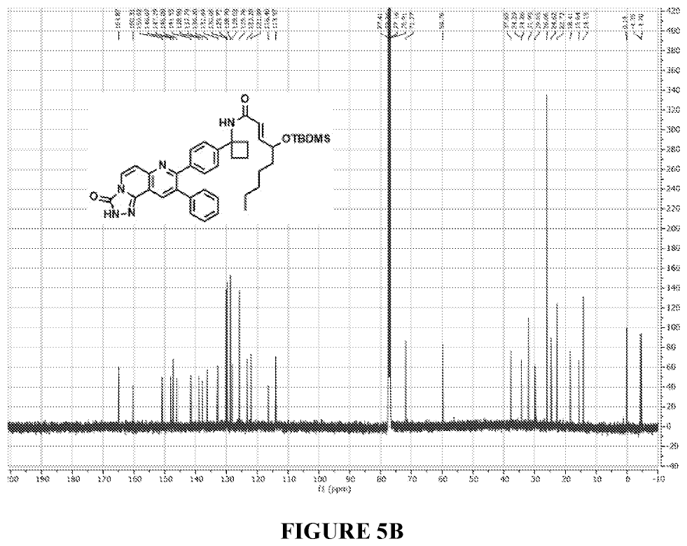

The coupling reaction was followed general procedure described in Example 2. The crude product was purified by flash chromatography (from 4:1 v/v, toluene:Et₂OAc) to afford the desired product as a white solid (513 mg, 40%). ¹H NMR (500 MHz, CDCl₃) δ 9.05 (s, 1H), 8.50 (s, 1H), 7.78 (d, J=7.6 Hz, 1H), 7.43 (d, J=8.3 Hz, 2H), 7.39 (d, J=8.3 Hz, 2H), 7.34-7.30 (m, 3H), 7.28-7.27 (m, 2H), 7.07 (d, J=7.7 Hz, 1H), 6.78 (dd, J=15.0, 4.2 Hz, 1H), 5.88 (dd, J=15.0 Hz, 1H), 5.80 (s, 1H), 4.29 (m, 1H), 2.70-2.64 (m, 2H), 2.62-2.57 (m, 2H), 2.12-2.06 (m, 1H), 1.88-1.81 (m, 1H), 1.50 (t, J=7.5 Hz, 2H), 1.33-1.28 (m, 2H), 1.27-1.24 (s, 2H), 0.90 (s, 9H), 0.88 (t, J 5 Hz, 3H), 0.06 (s, 3H), 0.04 (s, 3H) (FIG. 5A). ¹³C NMR (125 MHz, CDCl₃) δ 164.87, 160.31, 150.82, 148.07, 147.29, 146.09, 141.55, 138.90, 137.79, 136.20, 132.84, 130.05, 129.72, 128.70, 128.02, 125.76, 123.28, 121.99, 116.40, 113.97, 59.76, 37.65, 34.29, 34.26, 31.99, 29.85, 26.06, 24.62, 22.72, 18.41, 15.64, 14.19, 0.14, −4.35, −4.70 (FIG. 5B).

### Synthesis of (E)-8-(4-(1-(4-Hydroxynon-2-enamido)cyclobutyl)phenyl)-3-oxo-9-phenyl-2,3-dihydro-[1,2,4]triazolo[3,4-f][1,6]naphthyridin-7-ium Chloride

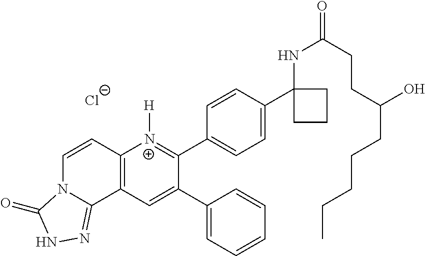 [SMILES: CCCCCC(O)CCC(=O)NC1(c2ccc(-c3[nH+]c4ccn5c(=O)[nH]nc5c4cc3-c3ccccc3)cc2)CCC1 ; [Cl-]]

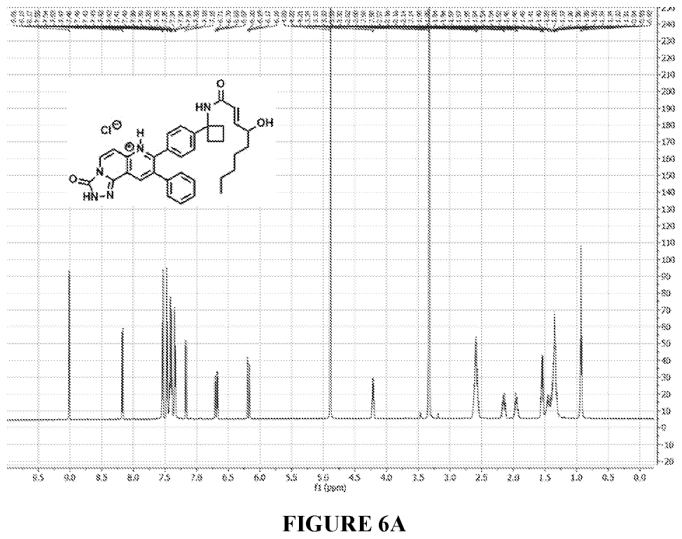

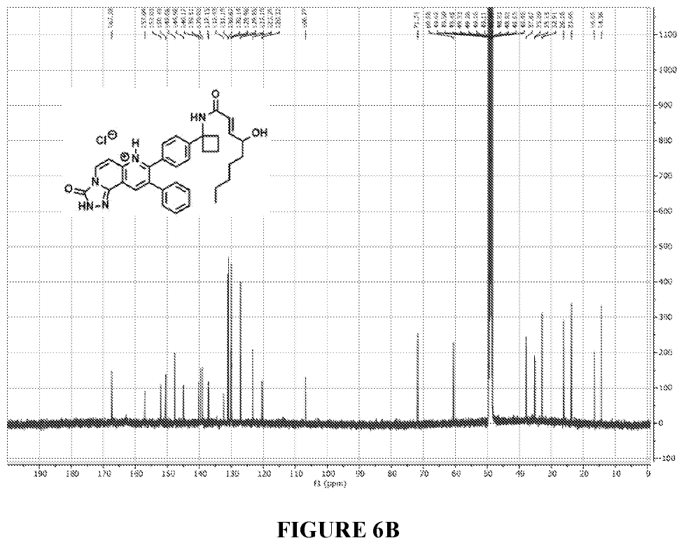

The resulting crude product was re-dissolved in CH₂Cl₂ and precipitated by slow addition of 1 M HCl in Et₂O to afford the desired product as yellow powder (408 mg, 90%). ¹H NMR (500 MHz,CD₃OD) δ 9.01 (s, 1H), 8.17 (d, J=7.6 Hz, 1H), 7.54 (d, J=8.3 Hz, 2H), 7.47 (d, J=8.4 Hz, 2H), 7.44-7.37 (m, 3H), 7.34-7.33 (m, 2H), 7.17 (d, J=7.7 Hz, 1H), 6.69 (dd, J=15.4, 5.1 Hz, 1H), 6.18 (dd, J=15.4, 1.6 Hz, 1H), 4.22 (app. q, J=6.0 Hz, 1H), 2.60-2.54 (m, 4H), 2.20-2.11 (m, 1H), 1.99-1.91 (m, 1H), 1.57-1.52 (m, 2H), 1.49-1.34 (m, 2H), 1.37-1.29 (m, 4H), 0.93 (t, J=6.8 Hz, 3H) (FIG. 6A). ¹³C NMR (125 MHz, CD₃OD) δ 167.38, 157.04, 152.03, 150.49, 147.66, 144.92, 140.17, 139.51, 139.02, 137.15, 132.43, 131.10, 130.83, 130.14, 129.98, 129.85, 127.10, 123.36, 120.32, 106.77, 71.74, 60.58, 35.20, 35.15, 32.91, 26.16, 23.66, 16.55, 14.36 (FIG. 6B).

### Example 6—Synthesis of (E)-8-(4-(1-(4-Fluoronon-2-en-8-ynamido)cyclobutyl)phenyl)-3-oxo-9-phenyl-2,3-dihydro-[1,2,4]triazolo[3,4-f][1,6]naphthyridin-7-ium Chloride (MK-FNE)

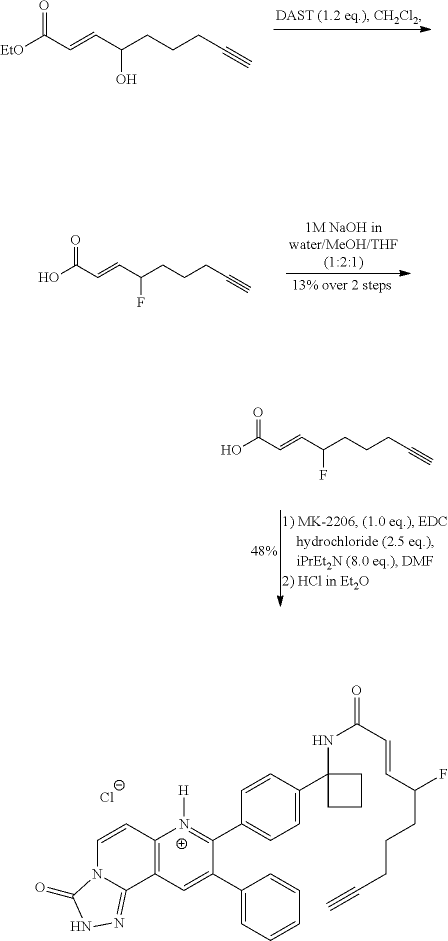 [SMILES: C#CCCCC(O)/C=C/C(=O)OCC ; C#CCCCC(F)/C=C/C(=O)O ; C#CCCCC(F)/C=C/C(=O)O ; C#CCCCC(F)/C=C/C(=O)NC1(c2ccc(-c3[nH+]c4ccn5c(=O)[nH]nc5c4cc3-c3ccccc3)cc2)CCC1 ; [Cl-]]

### Synthesis of (E)-4-Fluoronon-2-en-8-ynoic Acid

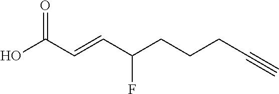

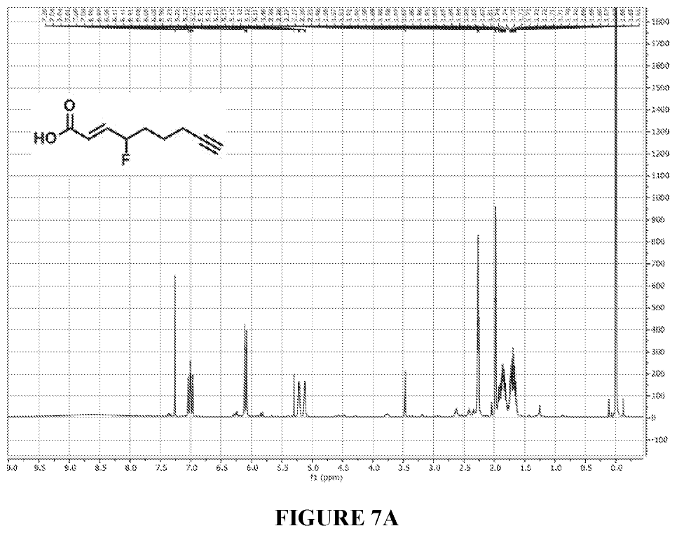

To a solution of ester (ethyl (E)-4-hydroxynon-2-en-8-ynoate) (125 mg, 0.64 mmol, 1.0 eq.) in CH₂Cl₂ (2 mL) was added DAST (100 μL, 123 mg, 0.77 mmol, 1.2 eq.) in CH₂Cl₂ (2 mL) dropwise. The resulting solution was stirred for 5 min before addition of sat. NaHCO₃ solution (5 mL). The organic fraction was separated, dried over Na₂SO₄, and evaporated in vacuo. The desired ester was separated from most by-products through flash chromatography (20:1 v/v, hexane/Et₂O). The resulting product was dissolved in MeOH/THF (2:1 v/v, 9 mL) followed by slow addition of 4 M NaOH solution (3 mL). The hydrolysis reaction was monitored by TLC analysis. After completion, 3 M HCl solution was added dropwise until pH reach 2.0. The resulting solution was extracted with Et₂O (3×20 mL) and the organic fractions were combined, dried, and evaporated. The desired product was afforded as a colorless oil (14 mg, 13%) through flash chromatography (20:1 v/v, hexane/Et₂O with 1% AcOH). ¹H NMR (500 MHz, CDCl₃) δ 7.00 (ddd, J=21.1, 15.7, 3.8 Hz, 1H), 6.10 (dt, J=15.6 Hz, 1H), 5.19 (m, 1H), 2.27 (td, J=6.9, 2.6 Hz, 2H), 1.98 (t, J=2.7 Hz, 1H), 1.94-1.80 (m, 2H), 1.70 (m, 2H) (FIG. 7A). ¹³C NMR (125 MHz, CDCl₃) δ 171.15, 147.53, 147.39, 120.68, 120.59, 91.43, 90.04, 83.49, 76.91, 69.29, 33.61, 33.45, 23.53, 23.50, 18.19 (FIG. 7B).

### Synthesis of MK-FNE

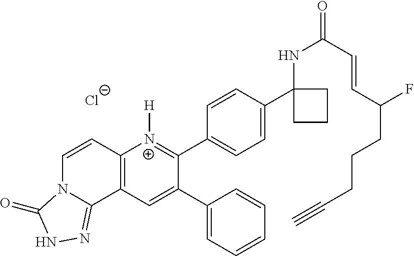 [SMILES: C#CCCCC(F)/C=C/C(=O)NC1(c2ccc(-c3[nH+]c4ccn5c(=O)[nH]nc5c4cc3-c3ccccc3)cc2)CCC1 ; [Cl-]]

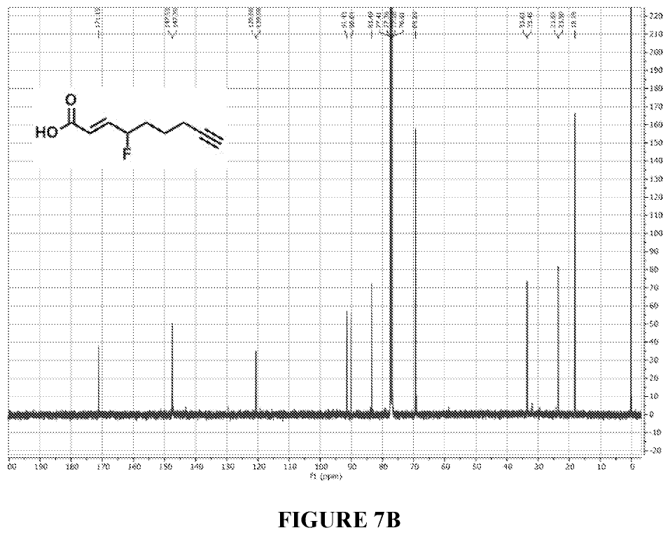

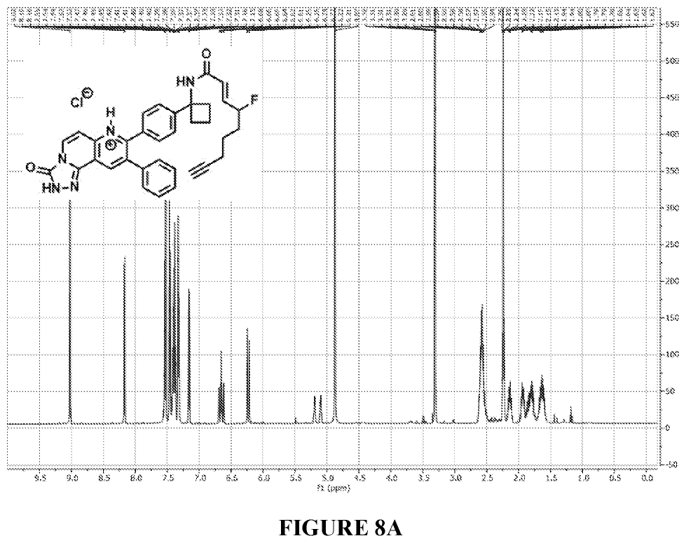

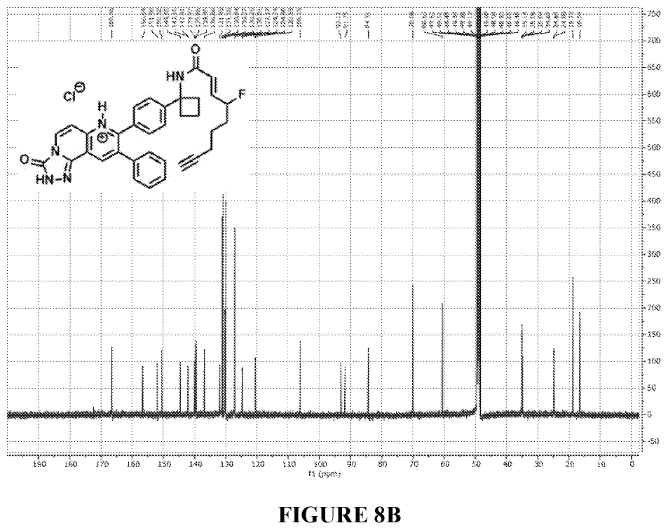

The coupling reaction followed the general procedure described in Example 2. The crude product was first purified by flash chromatography (from 4:1 v/v, toluene:Et₂OAc) followed by HCl precipitation to afford the desired product as yellow powder (12 mg, 48%). ¹H NMR (500 MHz, CD₃OD) δ 9.02 (s, 1H), 8.17 (d, J=7.6 Hz, 1H), 7.53 (d, J=8.0 Hz, 2H), 7.46 (d, J=8.4 Hz, 2H), 7.42-7.36 (m, 3H), 7.33-7.31 (m, 2H), 7.16 (d, J=7.7 Hz, 1H), 6.65 (ddd, J=19.9, 15.5, 4.4 Hz, 1H), 6.23 (dt, J=15.6, 1H), 5.09 (m, 1H), 2.64-2.51 (m, 4H), 2.27-2.20 (m, 3H), 2.18-2.09 (m, 1H), 1.98-1.89 (m, 1H), 1.88-1.74 (m, 2H), 1.63 (m, 2H) (FIG. 8A). ¹³C NMR (125 MHz, CD₃OD) δ 166.46, 156.64, 151.98, 150.52, 144.56, 142.16, 142.01, 139.97, 139.66, 139.46, 136.88, 131.95, 131.16, 130.84, 130.27, 130.25, 130.01, 127.17, 124.74, 124.66, 120.53, 106.15, 93.11, 91.75, 84.31, 70.08, 60.63, 35.14, 35.06, 35.04, 34.87, 24.84, 24.80, 18.73, 16.54 (FIG. 8B).

### Example 7—Synthesis of (E)-8-(4-(1-(Non-2-en-8-ynamido)cyclobutyl)phenyl)-3-oxo-9-phenyl-2,3-dihydro-[1,2,4]triazolo[3,4-f][1,6]naphthyridin-7-ium Chloride (MK-dHNE)

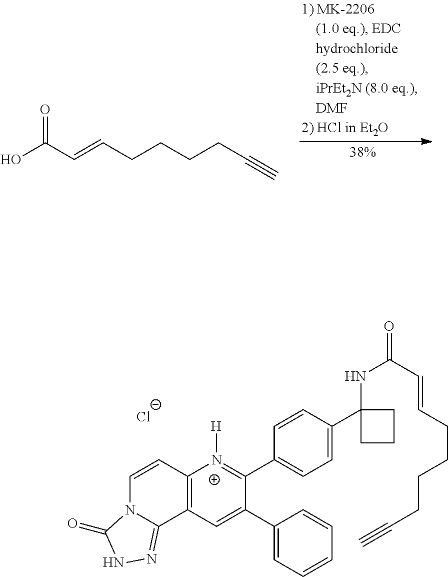 [SMILES: C#CCCCC/C=C/C(=O)O ; C#CCCCC/C=C/C(=O)NC1(c2ccc(-c3[nH+]c4ccn5c(=O)[nH]nc5c4cc3-c3ccccc3)cc2)CCC1 ; [Cl-]]

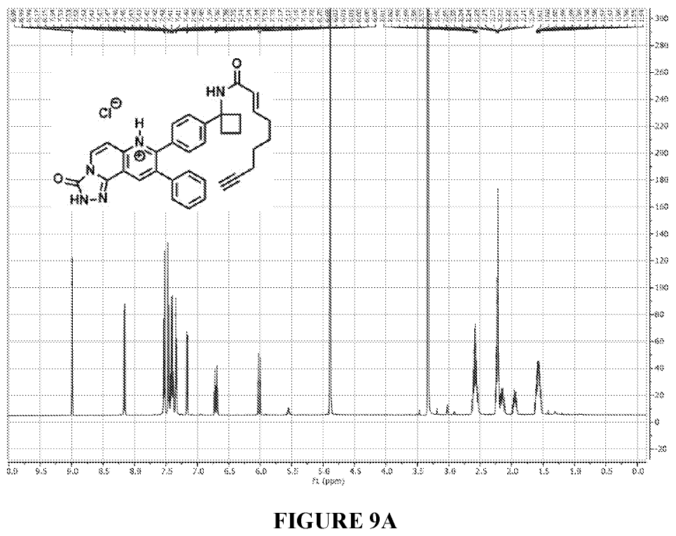

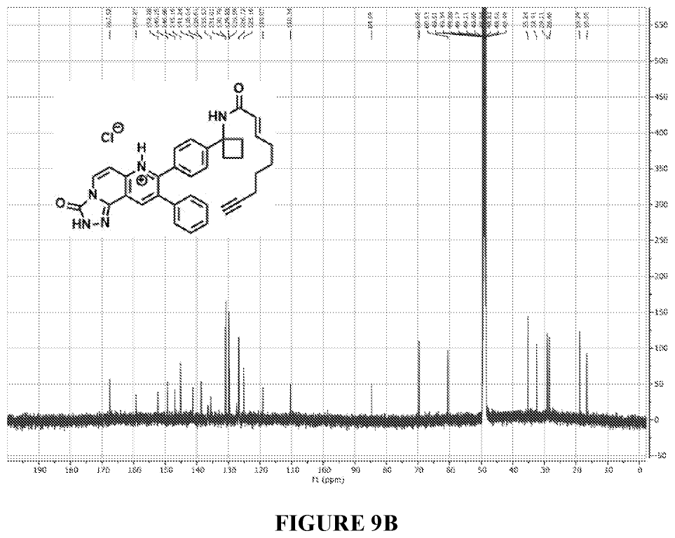

The coupling reaction followed the general procedure described in Example 2. The crude product was first purified by flash chromatography (from 4:1 v/v, toluene:EtOAc) followed by HCl precipitation to afford the desired product as yellow powder (10 mg, 38%). ¹H NMR (500 MHz, CD₃OD) δ 8.99 (s, 1H), 8.16 (d, J=7.7 Hz, 1H), 7.53 (d, J=8.5 Hz, 2H), 7.46 (d, J=8.5 Hz, 2H), 7.43-7.37 (m, 3H), 7.35-7.33 (m, 2H), 7.16 (d, J=7.8 Hz, 1H), 6.71 (dt, J=15.5, 7.0 Hz, 1H), 6.01 (dt, J=15.2, 1H), 2.65-2.52 (m, 4H), 2.28-2.17 (m, 5H), 2.14 (m, 1H), 1.95 (m, 1H), 1.65-1.50 (m, 4H) (FIG. 9A). ¹³C NMR (126 MHz, CD₃OD) δ 167.52, 159.27, 152.28, 149.25, 146.96, 145.16, 141.24, 138.64, 138.61, 135.53, 131.01, 130.79, 129.83, 129.59, 126.72, 125.16, 119.07, 110.34, 84.69, 69.68, 60.53, 35.24, 32.41, 29.11, 28.46, 18.79, 16.56 (FIG. 9B).

### Example 8—Synthesis of 8-(4-(1-(4-Hydroxynon-8-ynamido)cyclobutyl)phenyl)-3-oxo-9-phenyl-2,3-dihydro-[1,2,4]triazolo[3,4-f][1,6]naphthyridin-7-ium Chloride (MK-HNA)

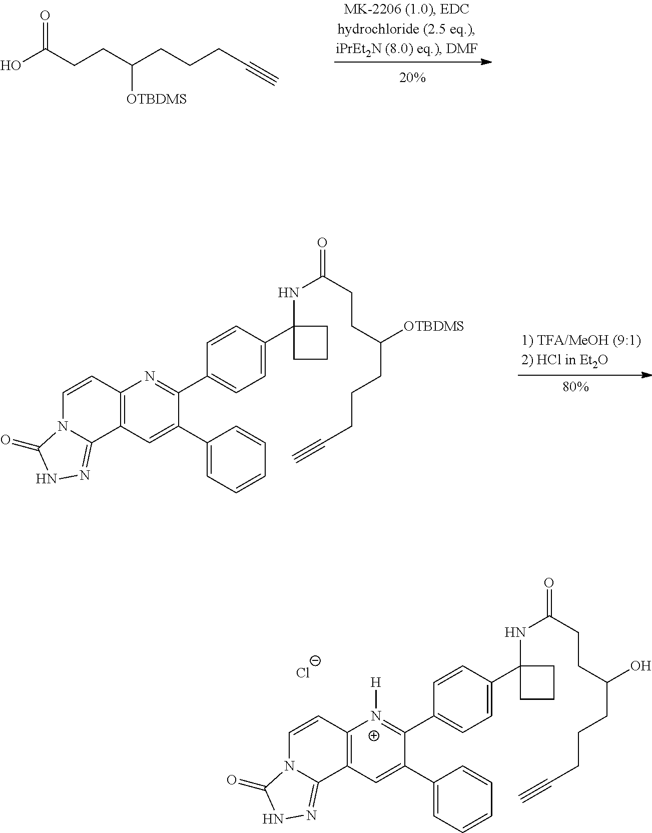 [SMILES: C#CCCCC(CCC(=O)O)O[Si](C)(C)C(C)(C)C ; C#CCCCC(CCC(=O)NC1(c2ccc(-c3nc4ccn5c(=O)[nH]nc5c4cc3-c3ccccc3)cc2)CCC1)O[Si](C)(C)C(C)(C)C ; C#CCCCC(O)CCC(=O)NC1(c2ccc(-c3[nH+]c4ccn5c(=O)[nH]nc5c4cc3-c3ccccc3)cc2)CCC1 ; [Cl-]]

### Synthesis of 4-((tert-Butyldimethylsilyl)oxy)-N-(1-(4-(3-oxo-9-phenyl-2,3-dihydro-[1,2,4]triazolo[3,4-f][1,6]naphthyridin-8-yl)phenyl)cyclobutyl)non-8-ynamide

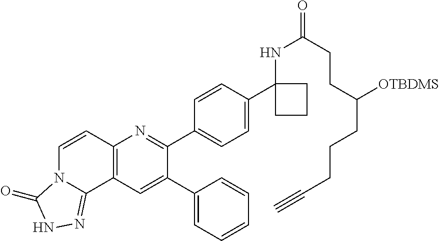 [SMILES: C#CCCCC(CCC(=O)NC1(c2ccc(-c3nc4ccn5c(=O)[nH]nc5c4cc3-c3ccccc3)cc2)CCC1)O[Si](C)(C)C(C)(C)C]

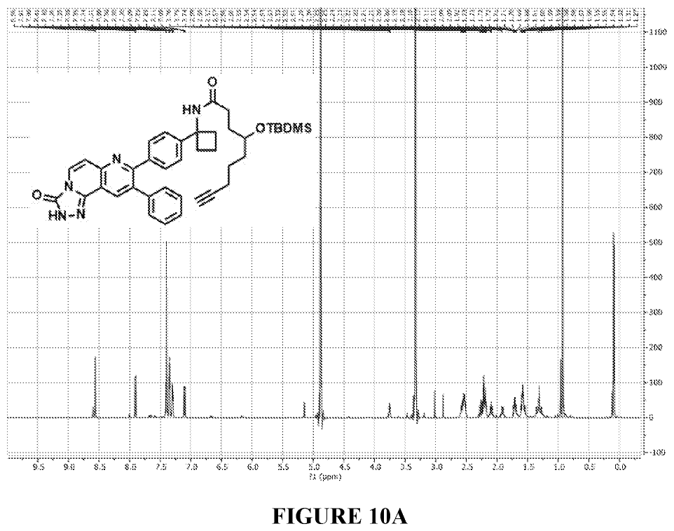

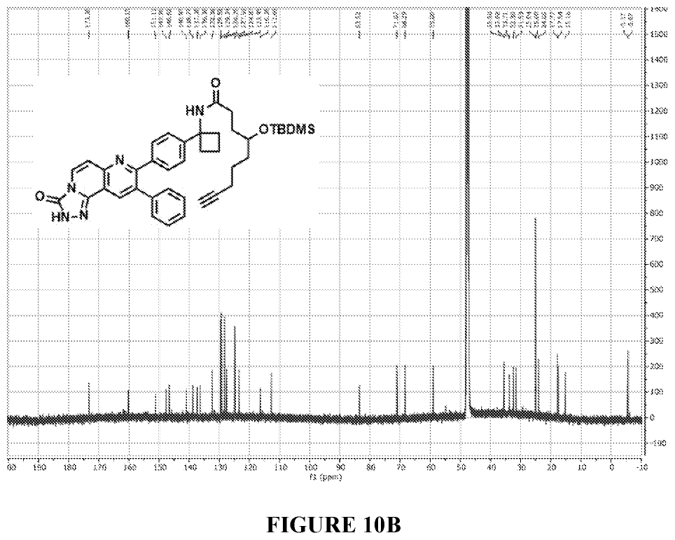

The coupling reaction followed the general procedure described in Example 2. The crude product was purified by flash chromatography (from 4:1 v/v, toluene:EtOAc) to afford the desired product as a white solid (10 mg, 20%). ¹H NMR (500 MHz, CDCl₃) δ 8.56 (s, 1H), 7.91 (d, J=7.6 Hz, 1H), 7.41-7.40 (m, 4H), 7.36-7.34 (m, 3H), 7.32-7.27 (m, 2H), 7.10 (d, J=7.6 Hz, 1H), 3.75 (m, 1H), 2.60-2.49 (m, 4H), 2.30-2.15 (m, 5H), 2.09 (m, 1H), 1.90 (m, 1H), 1.75-1.67 (m, 2H), 1.62-1.52 (m, 4H), 0.93 (s, 9H), 0.09 (s, 3H), 0.08 (s, 3H) (FIG. 10A). ¹³C NMR (126 MHz, CD₃OD) δ 173.30, 160.15, 151.13, 147.70, 146.62, 140.97, 138.77, 137.30, 136.30, 132.36, 129.52, 129.34, 128.25, 127.59, 124.87, 123.45, 116.36, 112.69, 83.52, 71.07, 68.29, 59.06, 35.50, 33.82, 33.71, 32.38, 31.53, 25.04, 25.00, 24.05, 17.77, 17.54, 15.16, −5.57, −5.67 (FIG. 10B).

### Synthesis of MK-HNA

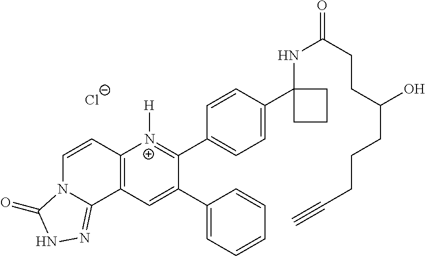 [SMILES: C#CCCCC(O)CCC(=O)NC1(c2ccc(-c3[nH+]c4ccn5c(=O)[nH]nc5c4cc3-c3ccccc3)cc2)CCC1 ; [Cl-]]

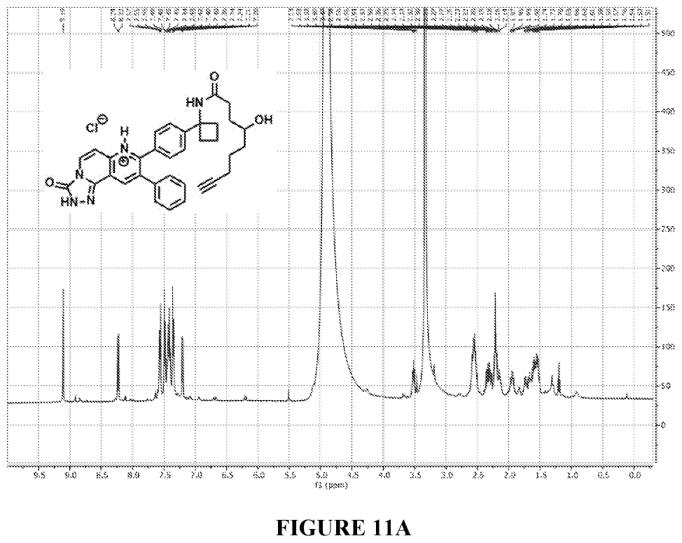

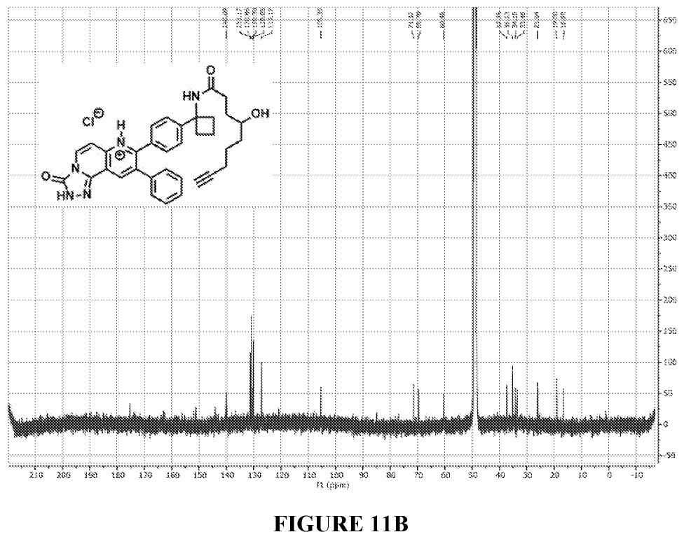

The silyl ether deprotection reaction followed the general procedure described in Example 3. The resulting crude product was re-dissolved in CH₂Cl₂ and precipitated by slow addition of 1 M HCl in Et₂O to afford the desired product as yellow powder (7 mg, 80%). ¹H NMR (500 MHz, CD₃OD) δ 9.10 (s, 1H), 8.23 (d, J=7.7 Hz, 1H), 7.56 (d, J=8.0 Hz, 2H), 7.49 (d, J=8.1 Hz, 2H), 7.46-7.40 (m, 3H), 7.35 (app. d, J=7.1 Hz, 2H), 7.20 (d, J=7.6 Hz, 1H), 3.51 (m, 1H), 2.59-2.49 (m, 4H), 2.38-2.25 (m, 2H), 2.24-2.18 (m, 3H), 2.14 (m, 1H), 1.94 (m, 1H), 1.78-1.63 (m, 2H), 1.62-1.50 (m, 4H) (FIG. 11A). ¹³C NMR (125 MHz, CD₃OD) δ 140.09, 131.17, 130.86, 130.39, 130.05, 127.17, 105.35, 71.37, 69.70, 60.48, 37.35, 35.13, 34.18, 33.46, 25.94, 19.00, 16.50 (FIG. 11B).

### Example 9—Synthesis of (E)-8-(4-(1-(4-Fluoronon-2-enamido)cyclobutyl)phenyl)-3-oxo-9-phenyl-2,3-dihydro-[1,2,4]triazolo[3,4-f][1,6]naphthyridin-7-ium Chloride (MK-FNE No Alkyne)

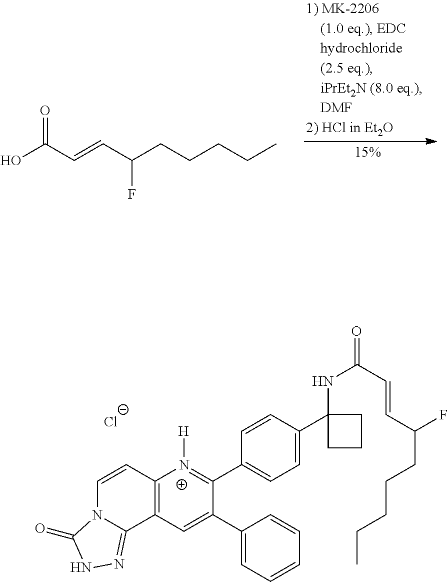 [SMILES: CCCCCC(F)/C=C/C(=O)O ; CCCCCC(F)/C=C/C(=O)NC1(c2ccc(-c3[nH+]c4ccn5c(=O)[nH]nc5c4cc3-c3ccccc3)cc2)CCC1 ; [Cl-]]

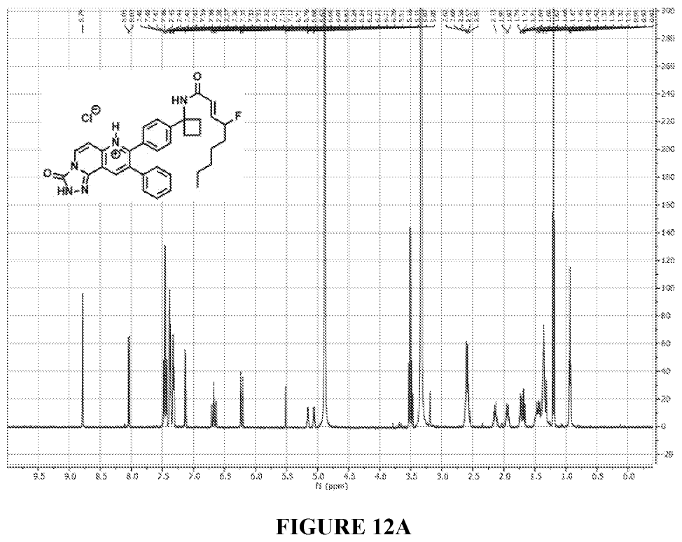

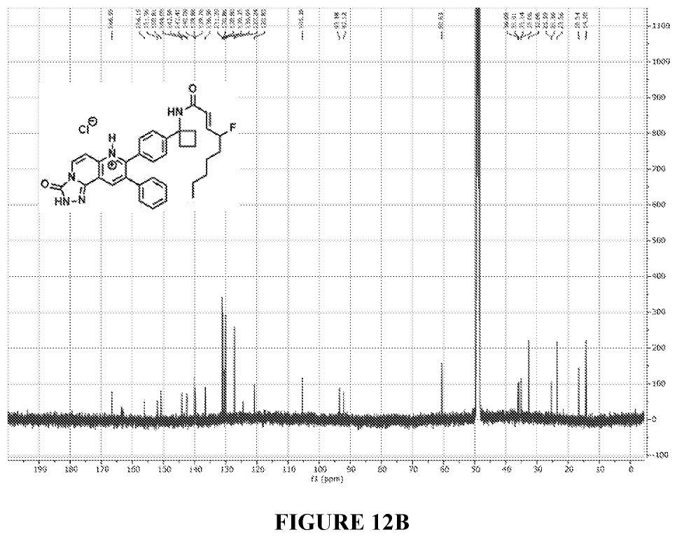

The coupling reaction followed the general procedure described in Example 2. The crude product was first purified by flash chromatography (from 4:1 v/v, toluene:Et₂OAc) followed by HCl precipitation to afford the desired product as a white solid (7 mg, 15%). ¹H NMR (500 MHz, CD₃OD) δ 8.79 (s, 1H), 8.04 (d, J=7.6 Hz, 1H), 7.47 (d, J=8.5 Hz, 2H), 7.44 (d, J=8.5 Hz, 2H), 7.39-7.36 (m, 3H), 7.33-7.31 (m, 2H), 7.13 (d, J=7.7 Hz, 1H), 6.67 (ddd, J=19.9, 15.5, 4.4 Hz, 1H), 6.22 (dt, J=15.5, 1H), 5.11 (m, 1H), 2.64-2.54 (m, 4H), 2.18-2.09 (m, 1H), 1.98-1.91 (m, 1H), 1.75-1.64 (m, 2H), 1.50-1.40 (m, 2H), 1.38-0.92 (m, 7H) (FIG. 12A). ¹³C NMR (125 MHz, CD₃OD) δ 166.59, 156.15, 151.96, 150.81, 144.09, 142.56, 142.41, 140.09, 139.88, 139.76, 136.56, 131.20, 130.86, 130.80, 130.35, 130.04, 127.24, 120.83, 105.38, 93.48, 92.12, 60.63, 36.08, 35.91, 35.14, 35.06, 32.66, 25.39, 25.36, 23.56, 16.54, 14.30 (FIG. 12B).

### Example 10—Synthesis of 8-(4-(1-(3-Hydroxy-2-methylenenon-8-ynamido)cyclobutyl)phenyl)-3-oxo-9-phenyl-2,3-dihydro-[1,2,4]triazolo[3,4-f][1,6]naphthyridin-7-ium Chloride (MK-G)

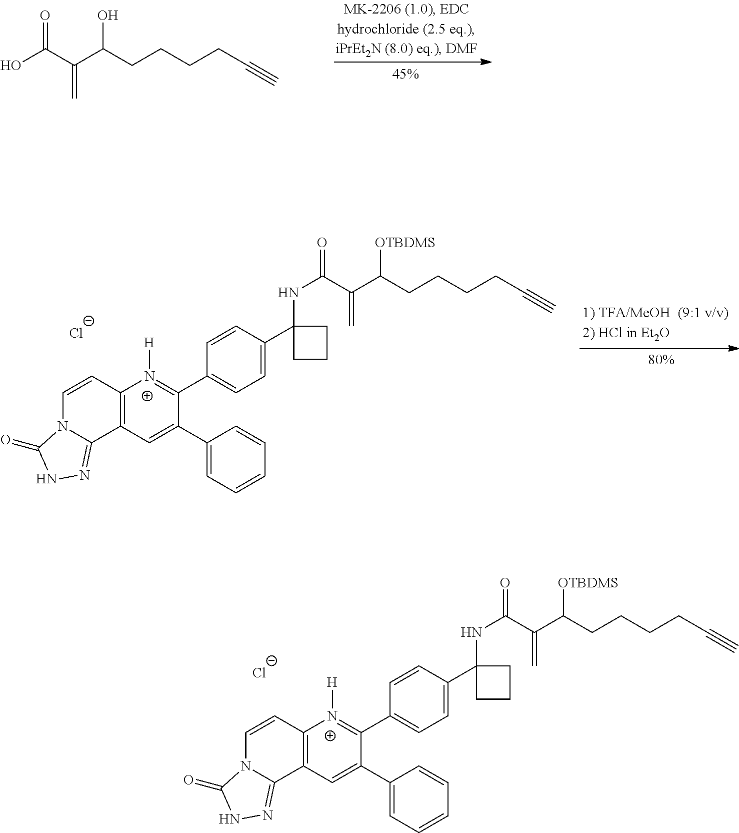 [SMILES: C#CCCCCC(O)C(=C)C(=O)O ; C#CCCCCC(O[Si](C)(C)C(C)(C)C)C(=C)C(=O)NC1(c2ccc(-c3[nH+]c4ccn5c(=O)[nH]nc5c4cc3-c3ccccc3)cc2)CCC1 ; [Cl-] ; C#CCCCCC(O[Si](C)(C)C(C)(C)C)C(=C)C(=O)NC1(c2ccc(-c3[nH+]c4ccn5c(=O)[nH]nc5c4cc3-c3ccccc3)cc2)CCC1 ; [Cl-]]

### Synthesis of 3-((tert-butyldimethylsilyl)oxy)-2-methylene-N-(1-(4-(3-oxo-9-phenyl-2,3-dihydro-[1,2,4]triazolo[3,4-f][1,6]naphthyridin-8-yl)phenyl)cyclobutyl)non-8-ynamide

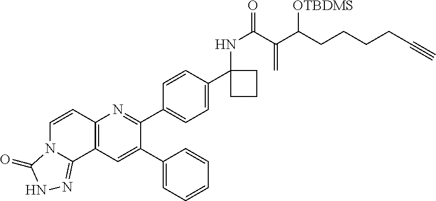 [SMILES: C#CCCCCC(O[Si](C)(C)C(C)(C)C)C(=C)C(=O)NC1(c2ccc(-c3nc4ccn5c(=O)[nH]nc5c4cc3-c3ccccc3)cc2)CCC1]

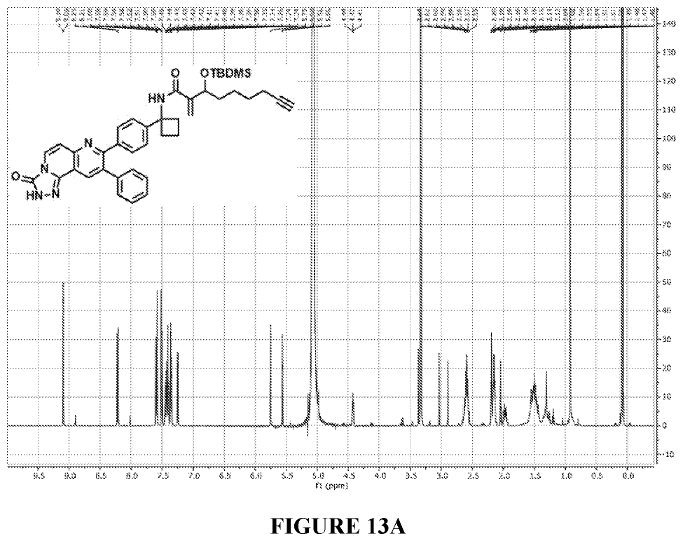

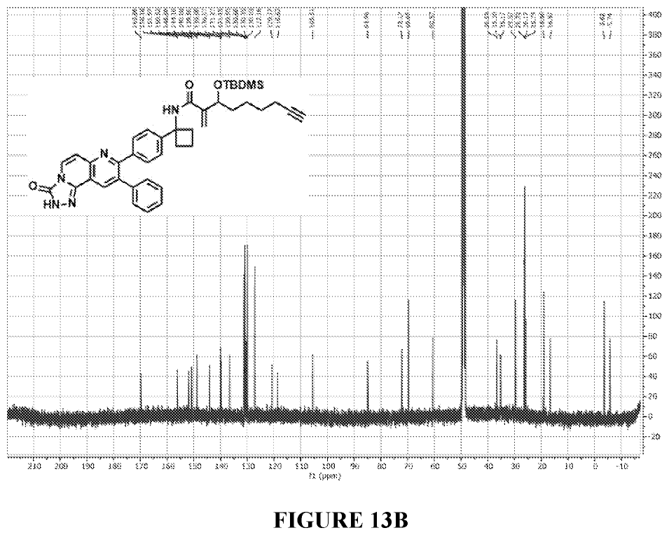

The coupling reaction followed the general procedure described in Example 2. The crude product was purified by flash chromatography (from 4:1 v/v, toluene:Et₂OAc) to afford the desired product as a white solid (10 mg, 45%). ¹H NMR (500 MHz, CD₃OD) δ 9.10 (s, 1H), 8.22 (d, J=7.7 Hz, 1H), 7.59 (d, J=8.5 Hz, 2H), 7.51 (d, J=8.5 Hz, 2H), 7.46-7.38 (m, 3H), 7.36-7.34 (m, 2H), 7.25 (d, J=7.5 Hz, 1H), 5.75 (s, 1H), 5.56 (s, 1H), 4.42 (m, 1H), 2.65-2.53 (m, 4H), 2.19 (t, J=2.6 Hz, 1H), 2.17-2.13 (m, 3H), 1.97 (m, 1H), 1.58-1.42 (m, 4H), 1.32-1.26 (m, 2H), 0.92 (s, 9H), 0.09 (s, 3H), 0.07 (s, 3H) (FIG. 13A). ¹³C NMR (125 MHz, CD₃OD) δ 169.86, 156.16, 151.97, 150.82, 148.88, 144.10, 140.08, 139.86, 139.80, 136.57, 131.27, 131.15, 130.85, 130.68, 130.35, 130.03, 127.16, 120.77, 118.63, 105.51, 84.96, 72.17, 69.65, 60.57, 36.59, 35.30, 35.17, 29.57, 26.32, 26.17, 25.74, 18.96, 16.57, −3.62, −5.74 (FIG. 13B).

### Synthesis of MK-G

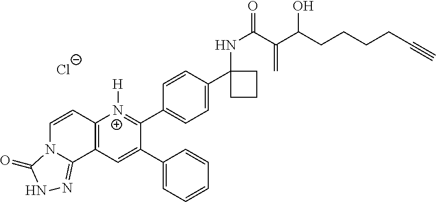 [SMILES: C#CCCCCC(O)C(=C)C(=O)NC1(c2ccc(-c3[nH+]c4ccn5c(=O)[nH]nc5c4cc3-c3ccccc3)cc2)CCC1 ; [Cl-]]

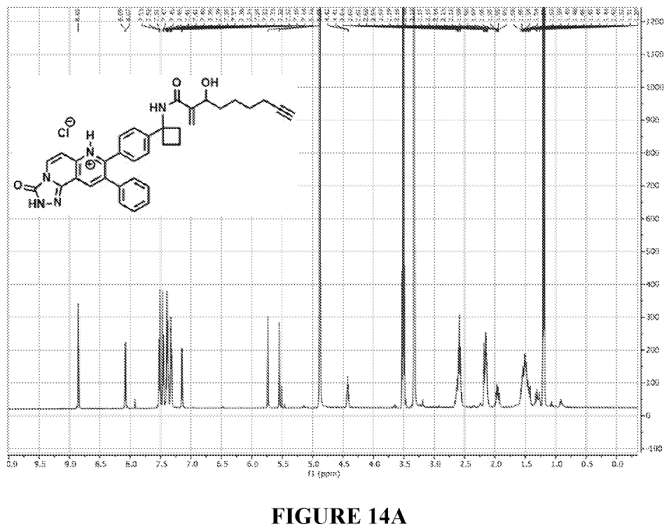

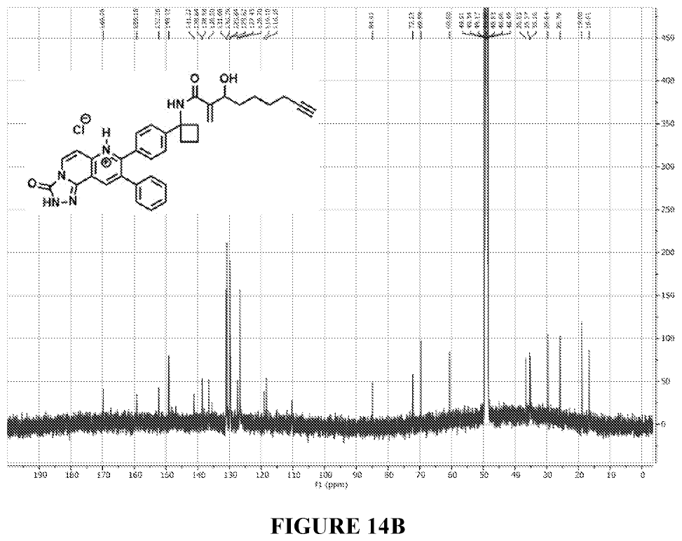

The silyl ether deprotection reaction followed the general procedure described in Example 3. The resulting crude product was re-dissolved in CH₂Cl₂ and precipitated by slow addition of 1 M HCl in Et₂O to afford the desired product as yellow powder (8.5 mg, 80%). ¹H NMR (500 MHz,CD₃OD) δ 8.85 (s, 1H), 8.08 (d, J=7.7 Hz, 1H), 7.52 (d, J=8.4 Hz, 2H), 7.46 (d, J=8.3 Hz, 2H), 7.43-7.35 (m, 3H), 7.35-7.30 (m, 2H), 7.15 (d, J=7.7 Hz, 1H), 5.74 (s, 1H), 5.55 (s, 1H), 4.42 (m, 1H), 2.65-2.54 (m, 4H), 2.18 (t, J=2.5 Hz, 1H), 2.16-2.12 (m, 3H), 1.95 (m, 1H), 1.57-1.40 (m, 4H), 1.34-1.27 (m, 2H) (FIG. 14A). ¹³C NMR (125 MHz, CD₃OD) δ 169.76, 159.16, 152.26, 149.12, 141.22, 138.64, 138.58, 136.50, 131.08, 130.79, 129.84, 129.62, 127.45, 126.70, 119.10, 118.35, 84.93, 72.23, 69.59, 60.60, 36.63, 35.37, 35.16, 29.64, 25.78, 19.00, 16.61 (FIG. 14B).

### Example 11—Cell Culture Conditions and Harvesting

HEK293T cells were cultured in MEM medium; all other cell lines mentioned in this application were cultured in DMEM medium. Complete medium in the following examples stands for the culture medium supplemented with 10% FBS and 1% of a stock solution containing 10,000 IU/ml penicillin, and 10,000 μg/ml streptomycin, 100 mM pyruvate (final concentration 1 mM) and non-essential amino acid (100×, 1140076, ThermoFisher Scientific). The cell culture was placed in an incubator at 37° C. under a humidified atmosphere containing 5% CO₂. Rinse medium was the corresponding complete medium without FBS. Cells were harvested with TrypLE™ Express (12605-028, Gibco® by Life Technologies™) followed by the addition of the corresponding complete medium to quench trypsin activity.

### Example 12—Cell Transfection

The HEK293T cells were split in a 6-well plate. After reaching 60% confluence, cells were transfected with 2.5 μg of the designated plasmid(s) in the stated ratios and 7.5 μL TransIT® 2020 in MEM medium.

### Example 13—Cell Lysis and Lysate Collection

Cells were lysed in 50 mM HEPES, 100 mM NaCl, 1 mM TCEP, and 1% Triton-X solution containing 1×Roche EDTA-free Protease inhibitor by rapid freeze-thaw (×3). Cell debris was removed and the supernatant was collected after centrifugation at 20,000×g for 10 min at 4° C. Protein concentration was determined using Bradford assay.

### Example 14—SDS-PAGE

10% SDS PAGE gels were prepared according to a protocol of Bio-Rad. The prepared samples were loaded into wells (˜35 μL for a 10-well lane), and electrophoresis was carried out using 120 V for 10 min followed by 150 V until the run was completed.

### Example 15—Western Blotting

The proteins in gel were transferred onto a PVDF membrane in Towbin buffer at 90 V for 2 hours at 4° C. or at 33 V overnight at 4° C. The membrane was blocked with 5% BSA in 100 mM Tris pH 7.6, 150 mM NaCl, 0.1% Tween (TBS-T1) and probed with various antibodies at the indicated dilutions (Table 1) typically in 1% BSA in TBS-T1 overnight in 4° C. cold room for primary; then washed 3 times in TBS-T1 for 10 min each; then probed with secondary antibody for 1 hour at room temperature; then washed two times in TBS-T1 followed by one wash in TBS. Proteins were visualized using Pierce™ ECL Western Blotting Substrate (32106, Thermo Fisher), Pierce™ ECL2 Western Blotting Substrate (32132, Thermo Fisher) or SuperSignal™ Western Blot Enhancer (46640, Thermo Fisher), depending on the signal strength.

TABLE 1
Dilutions and Incubation Time for Antibodies

| | | Incubation |
|---|---|---|
| Antibody (commercial source) | Dilution | time (h) |
| Anti-Halo monoclonal antibody | 1:1,000 in 1% | 16 |
| (Promega) | BSA in TBS-T1 | |
| Phospho-Akt (Thr308) antibody | 1:1,000 in 1% | 16 |
| (9275, Cell Signaling | BSA in TBS-T1 | |
| Technology) | | |
| Akt3 monoclonal antibody | 1:1,000 in 1% | 16 |
| (4059, Cell Signaling) | BSA in TBS-T1 | |
| Akt2 (D6G4) Antibody (3063, | 1:1,000 in 1% | 16 |
| Cell Signaling) | BSA in TBS-T1 | |
| Akt (pan) (C67E7) monoclonal | 1:1,000 in 1% | 16 |
| antibody (4691, Cell Signaling) | BSA in TBS-T1 | |
| Anti-AKT1 antibody [4D6] | 1:1,000 in 1% | 16 |
| (ab124341, Abcam) | BSA in TBS-T1 | |
| Monoclonal anti-β-actin- | 1:10,000 in 1% | 1 |
| peroxidase antibody (A3854, | milk in TBS-T1 | |
| Sigma Aldrich) | | |
| Monoclonal anti- | 1:10,000 in 1% | 1 |
| GAPDH-Peroxidase | milk in TBS-T1 | |
| antibody (G9295, Sigma Aldrich) | | |
| Anti-rabbit IgG, HRP-linked | 1:5,000 in 1% | 1 |
| Antibody (7074, Cell Signaling) | BSA in TBS-T1 | |
| Anti-mouse IgG, HRP-linked | 1:5,000 in 1% | 1 |
| Antibody (7076, Cell Signaling) | BSA in TBS-T1 | |

### Example 16—in-Gel Fluorescence Assay

Cell transfection with the designated plasmid encoding a given HaloTag-Akt fusion gene was carried out following the general procedure described in Example 12. After 20-24 hours, the transfection media were aspirated and replaced with fresh 2 mL rinse MEM medium supplemented with 3% FBS containing the indicated concentration of inhibitor or the equivalent final volume of DMSO and the plates were incubated for the indicated period of time. Cells were then harvested and lysed following the general procedure described in Examples 11 and 13. A portion of the lysate was made up to 25 μL final volume containing, in final concentrations, 5% t-BuOH, [2 mM TCEP, 1% SDS, 1 mM CuSO₄, 0.1 mM Cu(TBTA), 10 μM Cy5 azide made up as a 10× stock in water] and 1.0 mg/mL lysate protein. The samples were incubated at 37° C. for 30 min and subsequently quenched with 8 μL of 4×Laemmeli dye containing 6% f ME. After an additional 5-min incubation at 37° C., samples were resolved on a 10% SDS-PAGE gel following the general procedure described in Example 14. After electrophoresis, the gel was rinsed three times with ddH₂O and imaged on a Bio-Rad Chemi-doc-MP Imager. Where applicable, the gel was transferred to a PVDF membrane for western blot analysis following the general procedure described in Example 15.

### Example 17—Biotin-Azide Pulldown of Endogenous Akt Kinases from Mammalian Lysate

HEK293T cells were split in 100 cm² plates. After reaching 60% confluence, media were aspirated and replaced with fresh 10 mL rinse media with 3% FBS and 5 μM MK-1-NE in DMSO or the equivalent volume of DMSO for 12 hours. Cells were harvested, washed twice with chilled 1×DPBS and flash frozen. Cell lysis was performed in 200 μL lysis buffer following general procedure described in Example 13, Each lysate was subsequently diluted to 2 mg/mL with lysis buffer and a small fraction of each lysate sample was kept for input gel. The remaining lysates were subjected to click reaction with biotin-azide for 30 min at 37° C. The final concentrations of each component was: 2 mM TCEP, 5% t-BuOH, 1% SDS, 1 mM CuSO₄, 0.1 mM Cu(TBTA), 10 μM biotin-azide and 1.2 mg/mL lysate protein. The lysate proteins were precipitated by adding four volumes of ethanol pre-chilled at −20° C. The sample was vortexed and incubated at −80° C. overnight (or at least 4 hours). The precipitant was collected by centrifugation at 20,000×g for 30 min at 4° C. and washed twice with pre-chilled methanol then acetone. The pellet was resuspended in 25 μL 50 mM HEPES (pH 7.6), 8% LDS and 0.5 μM EDTA and dissolved by vortexing and heating at 42° C. for 5 min. LDS was diluted to a final concentration of 0.5% by diluting the sample with 375 μL 50 mM HEPES (pH 7.6) and added to 50 μL bed volume of streptavidin Sepharose beads pre-equilibrated with 50 mM HEPES (pH 7.6) and 0.5% LDS. The sample was incubated with beads for 2-3 hours at room temperature by end-over-end rotation after which the supernatant was removed after centrifugation at 500×g for 3 min. The beads were washed three times with 400 μL of 50 mM HEPES (pH 7.6) with 0.5% LDS with end-over-end rotation at room temperature for 30 min during each wash. Bound proteins were eluted by boiling the beads at 98° C. for 10 min with 30 μL of 4×Laemmeli dye containing 6% βME. The sample was subjected to SDS-PAGE and transferred to a PVDF membrane for western blot analysis.

### Example 18—FRET Assay in Live Mammalian Cells

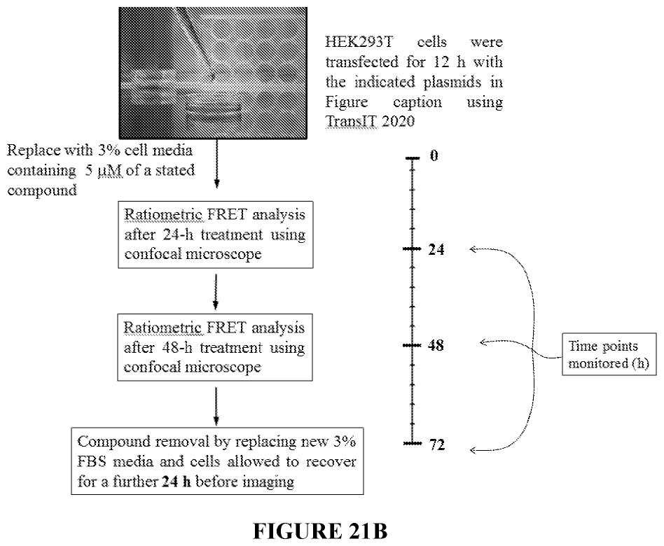

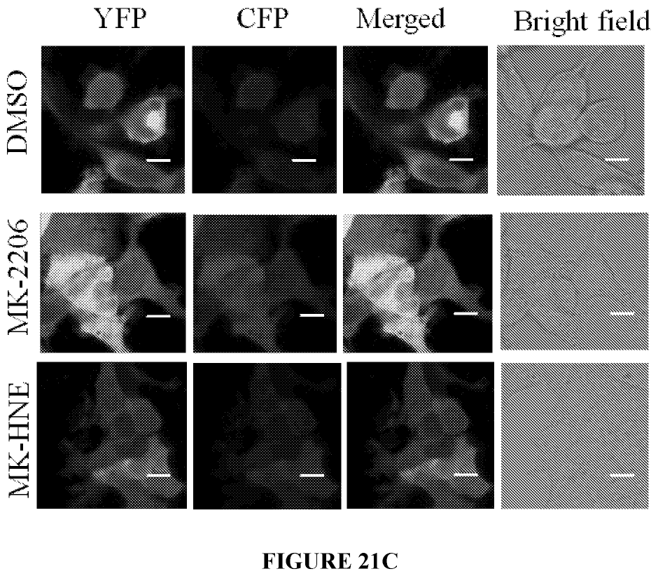

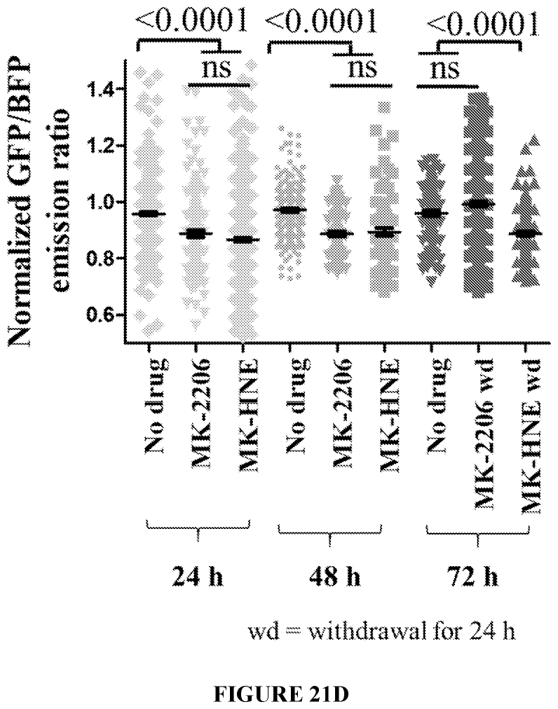

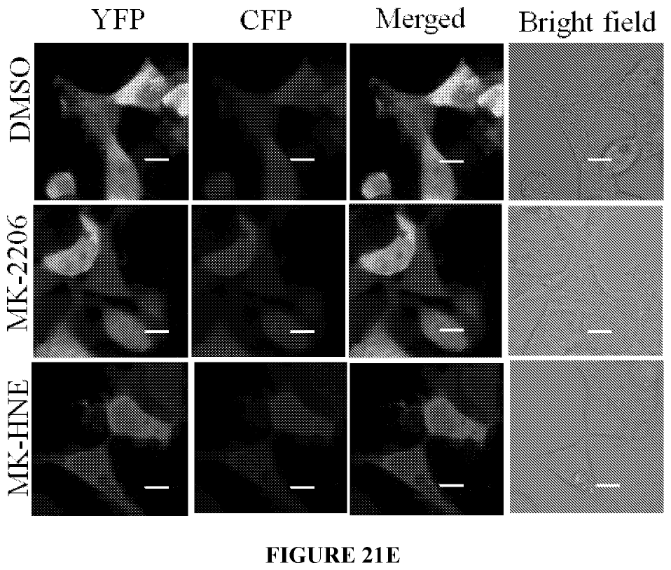

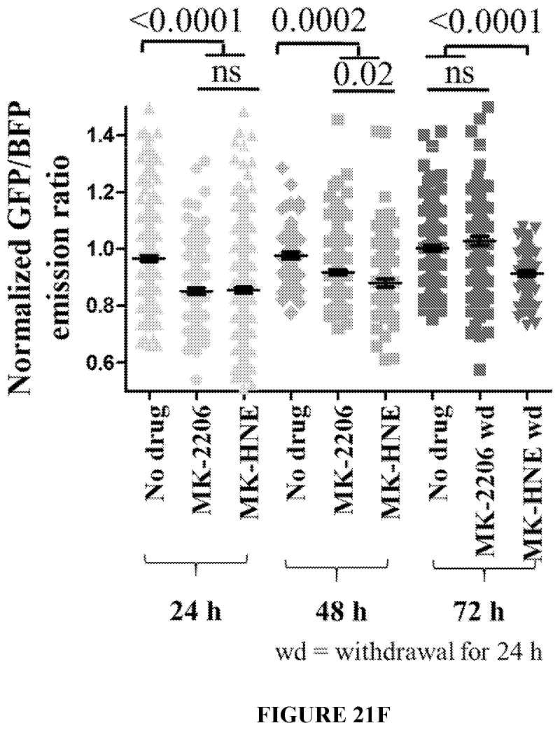

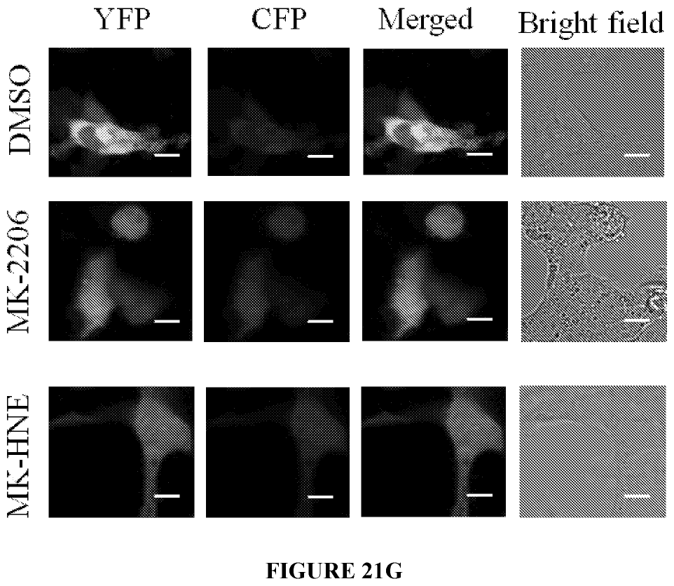

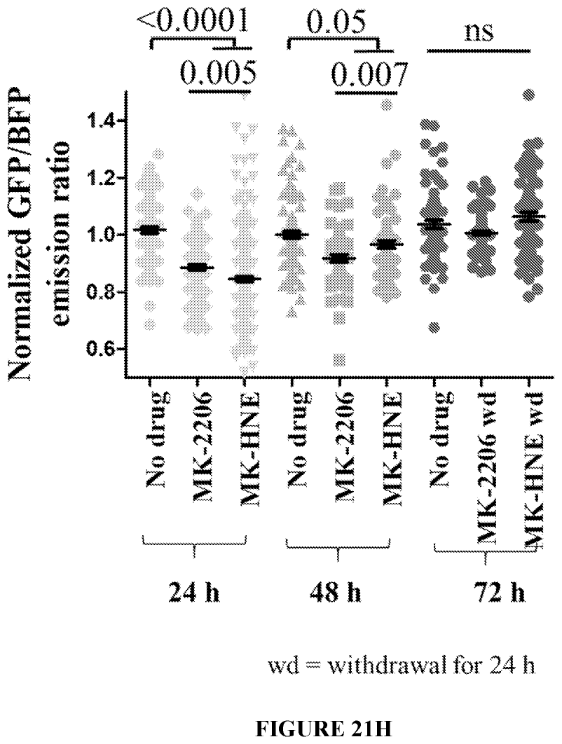

HEK293T cells were plated in imaging plates at 60% confluence. After 24 hours, cells in each plate were transfected with 1 μg AktAR reporter plasmid and 1 μg plasmid of the designated HaloTag fusion Akt gene in PCS2+8 vector, using TransIT 2020 (6 μL) (FIG. 21B). 12 hours after transfection, cells were treated with 5 μM inhibitor or the equivalent final volume of DMSO in rinse medium containing 3% FBS and incubated for 24 hours before imaging. FRET imaging was performed using a Zeiss LSM 710 confocal microscope as previously described. Briefly, a 458 nm argon laser was used for excitation. The signals in the cyan channel (463-498 nm) and the yellow channel (525-620 nm) were recorded. Cells were then incubated in drug medium for another 24 hours before next imaging. The medium was aspirated and the cells were gently rinsed with 3% FBS in rinse medium twice every 30 min over the next hour, Fresh rinse medium with 3% FBS was added and the cells were imaged again after 24 hours. For quantification, the mean CFP and YFP signal intensity was measured using Image by drawing a freehand circle around the cells, and the ratio image was calculated. Graphing and data analysis (Student's t-test) were performed using Prism software.

### Example 19—ELISA

Anti-Halo antibody at 1 μg/mL concentration in sodium bicarbonate buffer (pH 9.6) was added to a 96-well white plate (80 μL per plate) at 4° C. for 24 hours. After this, the incubation buffer was removed and the wells were washed once with TBS-T2 (100 mM Tris, 150 mM NaCl 0.03% Tween-20) and then blocked in 5% BSA in TBS-T2 (280 μL per well) for 3-5 hours at room temperature. After this time, BSA was washed away using TBS-T2, then wells were filled with 150 μL blocking buffer (1% BSA, 5 mM sodium orthovanadate, 20 mM NaF). Cells were lysed in lysis buffer (following the procedure described in Example 13 without the addition of TCEP in lysis buffer) with addition of 5 mM sodium orthovanadate, 20 mM NaF and 1×ROCHE complete minus EDTA protease inhibitors. 100 μg of each lysate (quantified by Bradford relative to BSA) was added to each well. This was shown to be the saturation conditions wherein the amount of phosphorylated protein detected reflects the ratio of phosphorylated to non-phosphorylated protein in the lysate. The mixture was incubated at 4° C. overnight. After this time, wells were washed with TBS-T2 three times, then primary antibody was added in 1% BSA in TBS-T2 overnight at 4° C. After this time, the wells were washed and HRP-conjugated secondary antibody was added in 1% milk in TBS-T2. After 1 hour at room temperature, wells were washed three times with TBS-T2 for 15 min then once with TBS for 20 min, after which 50 μL TBS was added to each well. HRP was detected using an autoinjector program on the Cytation 3 plate reader (Biotek). Femto ELISA substrate was used, injecting 50 μL Femto ELISA substrates 1 and 2 per well. Signals were calculated relative to that of a well coated in antibody and treated with untransfected lysate.

### Example 20—NADH-Coupled Akt Activity Assay

To a 96-well plate with varying amounts of indicated inhibitors was added the kinetic assay mixture containing, in final concentrations, 50 mM HEPES, 100 mM NaCl, 10 mM MgCl₂, 5 mM phosphoenolpyruvate, 500 μM NADH, 100 μM crosstide (the Akt substrate, cat. No. sc-471145, Santa Cruz), pyruvate kinase (24-40 units/mL), lactate dehydrogenase (36-56 units/mL), 4 mM TCEP, 500 μM ATP. The solution was allowed to equilibrate at room temperature for 5 min followed by the addition of Akt3 enzyme (0.3 jag, 100 nM). The resulting solution was mixed thoroughly by pipetting. The progress of phosphorylation was monitored by reading absorbance at 340 nm over the course of study. Curve fitting and data analyses were performed using Prism software.

### Example 21—Cell Viability Assay

Indicated cells were plated in a 96-well plate containing the DMEM complete medium (see Example 11 for complete medium formulation, 100 μL per well) with a density of 3,000-5,000 cells per well for 12 hours followed by the addition of varying concentrations of inhibitors in rinse medium (100 μL). The cells were allowed to grow for 72 hours and 100 μL of the culture medium was removed from each well. This was followed by the addition of 10 μL AlamarBlue® reagent to each well. The fluorescence emission signals at 590 nm were read by Cytation 3 cell imaging multi-mode reader (BioTeK) using an excitation wavelength of 560 nm. Curve fitting and data analysis were performed using Prism software.

### Example 22—Drug Effect Persistence Assay

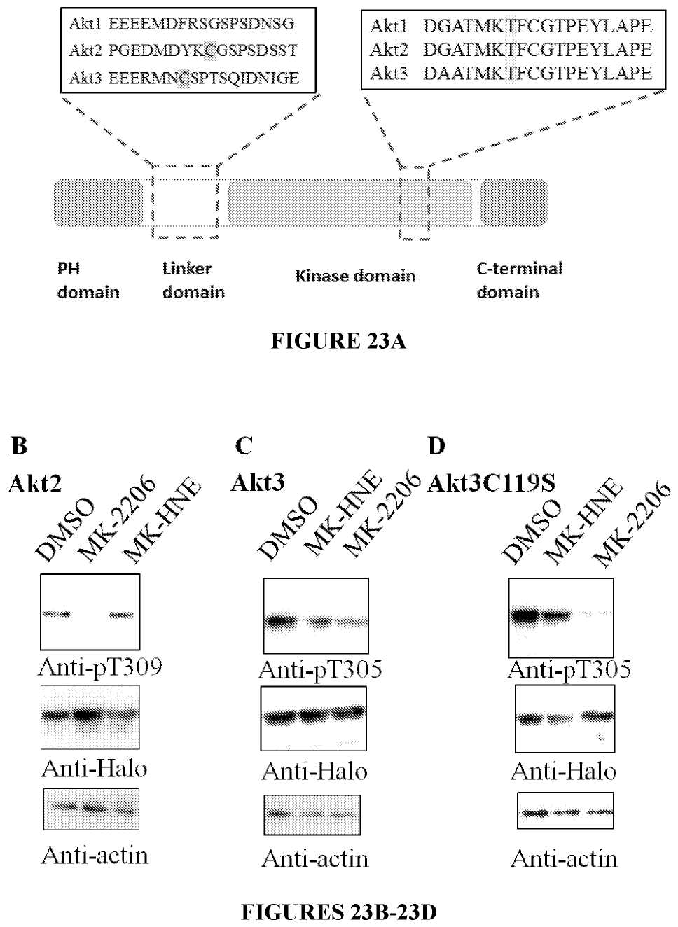

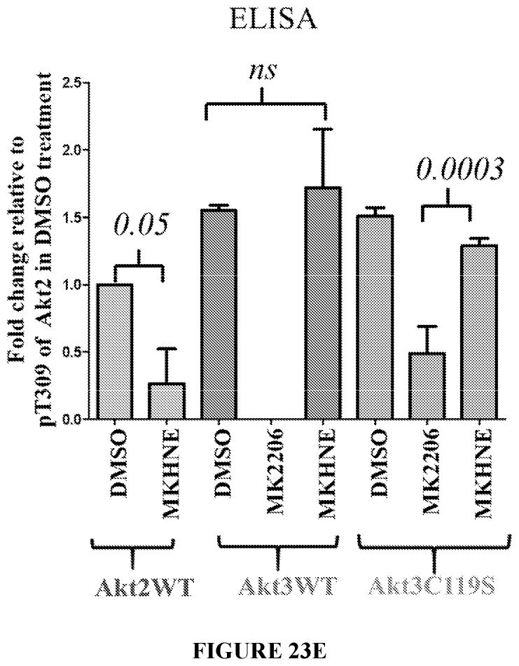

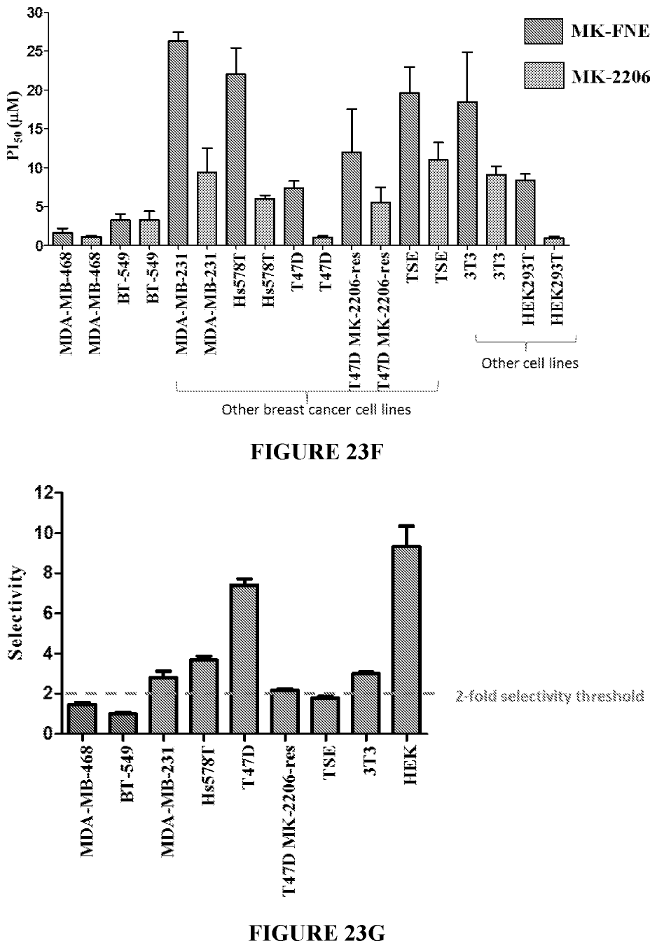

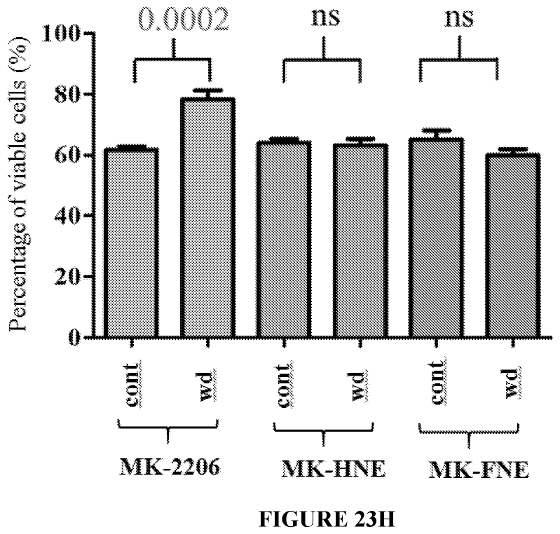

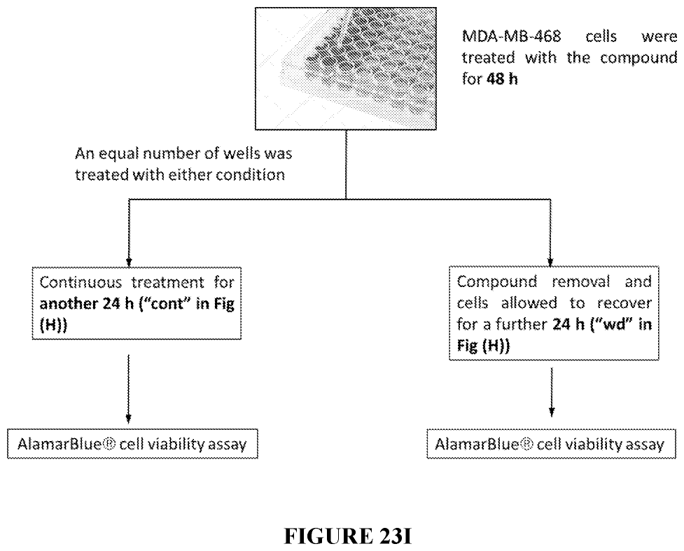

MDA-MB-468 cells were plated in a 96-well plate at a density of 3,000-5,000 cells per well for 10 hours before the addition of varying concentrations of inhibitors. The cells were allowed to grow for 48 hours. The cell medium was aspirated and the cells were washed once with DMEM medium. The cells continued to grow in 5% FBS DMEM medium for 24 hours before adding 1/10th volume of AlamarBlue® reagent to cells in culture medium. The fluorescence emission signals at 590 nm were read using a Cytation 3 cell imaging multi-mode reader (BioTeK) using an excitation wavelength of 560 nm. Curve fitting and data analyses were performed using Prism software (FIG. 23I).

### Example 23—Assay for Drug Synergism with Akt Isoform-Specific Knockdown

MDA-MB-468 cells were plated in a 6-well plate to reach 80% confluence 10 hours post seeding. At this time, cells were transfected with 75 pmol siRNA selectively targeting the indicated Akt isoform or control siRNA using lipofectamine 3000 (7.5 μL) (Tables 2-3), After 12 hours, culture medium was aspirated and the cells were harvested and plated in a 96-well plate at a density of 3,000-5,000 cells per well in complete culture medium. The cells were allowed to grow for 12 hours before addition of an equivalent volume of rinse medium containing varying concentrations of MK-2206, MK-FNE, or DMSO. The cells continued to grow for 72 hours before adding 1/10th volume of AlamarBlue® reagent to culture medium. The fluorescence emission signals at 590 nm were read by Cytation 3 cell imaging multi-mode reader (BioTeK) using an excitation wavelength of 560 nm. Curve fitting and data analysis were performed using Prism software. The effect of the different knockdowns on the drug action was expressed by calculating the fold selectivity (S):

Fold Selectivity={MK-FNE:[Average of EC₅₀(siConts)]/EC₅₀(siAktX)}÷{MK-2206:[Average of EC₅₀(siConts)]/EC₅₀(siAktX)}

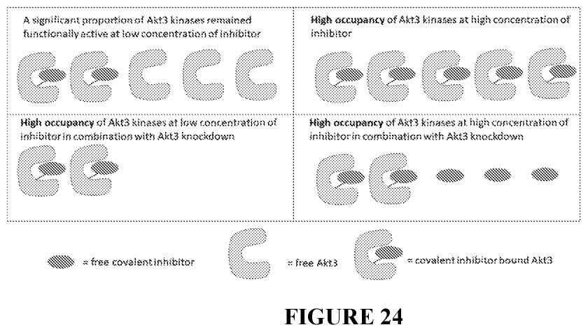

Using this metric, if knockdown of a protein selectively sensitizes to M K-FNE treatment a number greater than 1 will be observed, whereas if knockdown of a specific protein selectively sensitizes to MK-2206 a number less than 1 will be observed, siAKT1 selectively sensitized cells to MK-2206 (Fold Selectivity=0.5 and 0.4 respectively), consistent with Akt1 being the principal target of MK-2206 and a less important target for Akt3. Two different siAkt2 siRNAs gave different outputs, both of which were modest, indicating Akt2 may be targeted by each drug similarly, or Akt2 is not particularly important to this line. Remarkably, siAKT3 selectively sensitized cells to MK-FNE (Fold Selectivity=3.9 and 3.7). These results confirmed that in cells, MK-FNE is a potent and selective Akt3 inhibitor and is substantially different in a pharmaceutical spectrum from MK-2206. FIG. 24 shows a rational for these observed effects.

TABLE 2
Theromofisher siRNA Sequence

| | sense | antisense |
|---|---|---|
| siAkt1 | GGCUCCCCUCAACAACUUCTT | GAAGUUGUUGAGGGGAG |
| (42811) | (SEQ ID NO: 7) | CCTC (SEQ ID NO: 8) |
| siAkt2 | GGAUGAAGUCGCUCACACATT | UGUGUGAGCGACUUCAU |
| (103305) | (SEQ ID NO: 9) | CCTT (SEQ ID NO: 10) |
| siAkt3 | GGACCGCACACGUUUCUAUTT | AUAGAAACGUGUGCGGU |
| (110901) | (SEQ ID NO: 11) | CCTC (SEQ ID NO: 12) |

TABLE 3
Dharmacon siRNA for Akt3 Sequences

| Catalog number | Sequence |
|---|---|
| (J-003002-14-0002) | GAAGAGGGGAGAAUAUAUA |
| | (SEQ ID NO: 13) |
| (J-003002-16-0002) | GACAGAUGGCUCAUUCAUA |
| | (SEQ ID NO. 14) |

### Example 24—Cell Cycle Analysis Using Flow Cytometry

MDA-MB-468 cells in a 6-well plate at 10% confluence were treated with HNE alkyne or the indicated drug at PI₆₀ or PI₈₀ concentration for 72 hours at 37° C. Negative control contained the equivalent volume of DMSO. Cells were grown and analyzed in separate batches of 1-2 replicates per condition that were run 2-3 times each. Each time the data points were normalized to the control set for each batch. At the indicated time post compound treatment, the culture medium was aspirated and the adherent cells were detached from the plate using trypsin, quenched with complete media, centrifuged (700 g), washed once with PBS, centrifuged (700 g), resuspended in PBS, then fixed with 70% ethanol for 24 hours. The cell suspensions were spun down at 1,000 g and washed with 500 μl of PBS twice. Each cell pellet was treated with 500 μL staining solution containing 50 μl of Ribonuclease A (≥70 Kunitz units/mg proteins, R6513, Sigma-Aldrich) and 50 μl of a propidium iodide (1 mg/ml, Sigma-Aldrich) solution in PBS with 1% BSA. The resulting solution was incubated with agitation at room temperature for 30 min in the dark.

The stained cells were assayed on a BD LSR II flow cytometer. FlowJo® was used to deconvolute the signals. Cells were grouped first by forward and side scatter (area), then that group was regrouped using forward (width) versus forward (area) scatter to give single cells. Cells were than analyzed using cellular DNA content to quantitate the percentage of cells in the respective phases (G₁, S, G₂/M and sub-G₀) of the cell cycle.

### Example 25—Covalent Inhibition of Akt Requires the Enone Moiety

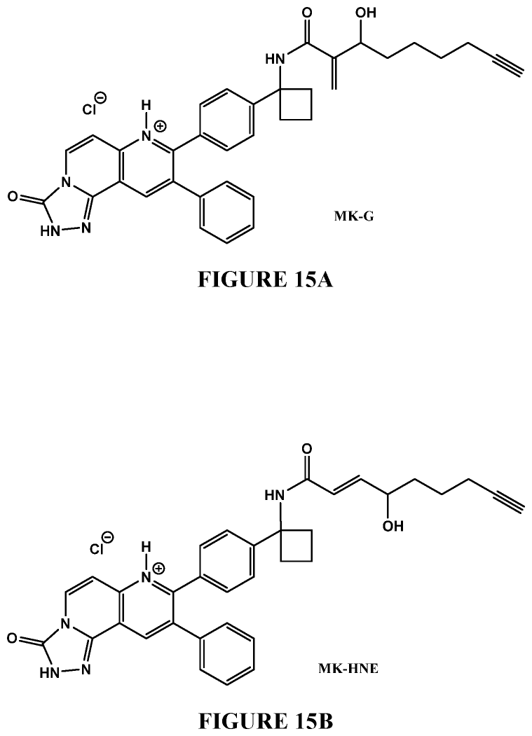

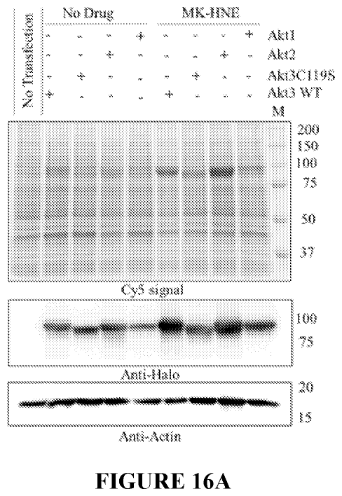

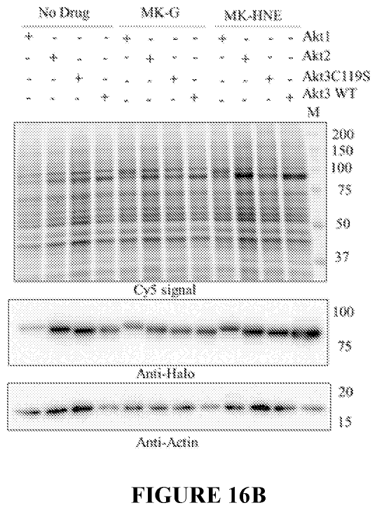

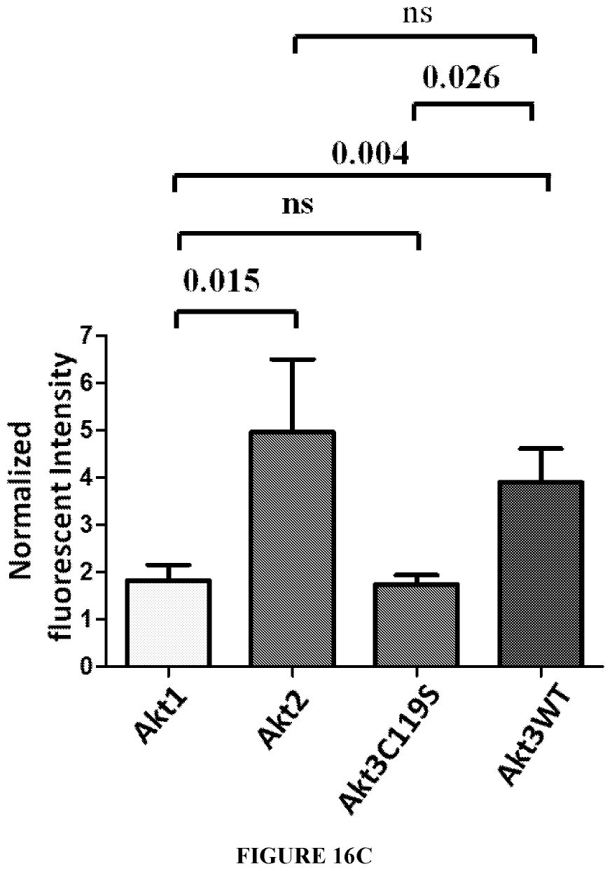

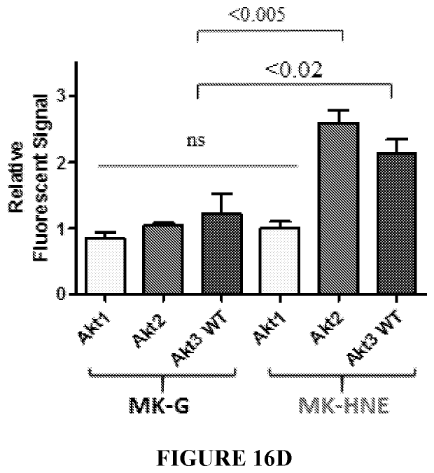

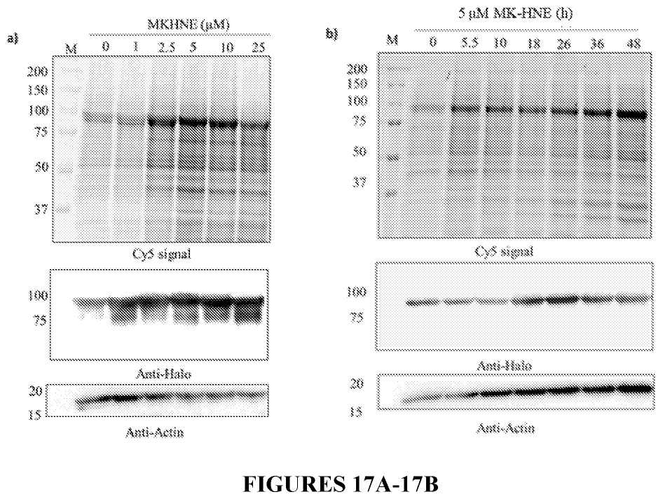

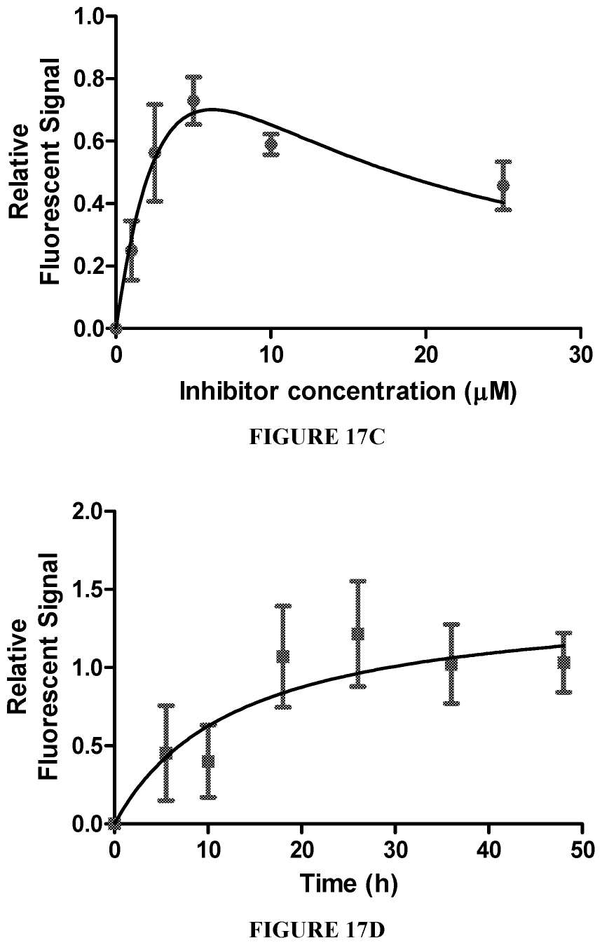

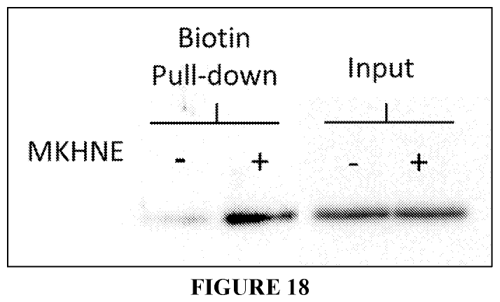

The reversible pan-Akt inhibitor MK-2206 was converted to an isozyme-specific irreversible/covalent inhibitor analogue (MK-HNE) by attaching an electrophilic appendage mimicking the native signaling lipid HNE (FIGS. 15A-15B, 16A-16D, 17A-17D, and 18). This design was aimed at specifically targeting innate redox-sensing cysteine residues C124 and C119 of Akt2 and Akt3, respectively, and achieving dominant-negative loss of function in terms of downregulating Akt2 and Akt3 oncogenic kinase activity. By contrast, no covalent inhibition by MK-HNE was found on Akt1 isoform (FIGS. 15A-15B, 16A-16D, and 17A-17D). Of equal importance, the C119S mutant of Akt3—that is defective for HNE sensing but otherwise functional for kinase activity—also showed no covalent modification by MK-HNE. These results were derived from orthogonal in vitro and cell-based assays, namely, kinetic activity assay with purified enzymes and fluorescent labeling in cell lysate. Importantly, a more reactive structural analogue of MK-HNE (called MK-G) containing a geminal-disubstituted alkene—in place of the trans-disubstituted alkene in MK-HNE—showed no isozyme-selective inhibition (FIGS. 15A-15B, 16A-16D, and 17A-17D). The selectivity underscores the virtue of the design, which relies on the structural resemblance between the lipid-electrophile-mimic appendage present within MK-HNE and the native redox signal HNE. These results further indicate that MK-HNE irreversibly interacts with the allosteric site of Akt2 and Akt3, likely mitigating off-target effects often elicited by the existing ATP-competitive kinase inhibitors (such as GSK690693 and AZD5363) (Nitulescu et al., “Akt Inhibitors in Cancer Treatment: The Long Journey From Drug Discovery to Clinical Use (Review),” Int. J. Oncol. 48(3):869-885 (2016), which is hereby incorporated by reference in its entirety). The current reversible pan-Akt inhibitors, namely, MK-2206, GSK690693, and inhibitor VIII, target Akt1 in favor of the other isoforms. By contrast, covalent inhibitor MK-HNE has a novel reverse inhibition profile (Akt inhibition efficiency: Akt3≈Akt2>>Akt1). Many modern pharmaceuticals, such as Orlistat and Afatinib, contain electrophilic appendages that either covalently hijack active-site nucleophiles, or target spectator nucleophilic side-chains which do not participate in enzymatic chemistry (Baillie, “Targeted Covalent Inhibitors for Drug Design,” Angew. Chem. Int. Ed. 55(43): 13408-13421 (2016), which is hereby incorporated by reference in its entirety). However, this invention suggests that innate electrophile-sensing residues are a quintessential resource for development of covalent drugs and the resulting Akt2- and Akt3-specific inhibition by MK-HNE can perturb the previously-untapped moonlighting roles of Akt kinase linked to redox sensing.

The reversible pan-Akt inhibitor (phase-II drug) MK-2206 was converted to an Akt3-isozyme-specific analogue (MK-HNE). This modification enabled such Akt inhibitors to possess a novel Akt-isoform selection panel, where the increment of selectivity follows: Akt1<<Akt2≈Akt3.

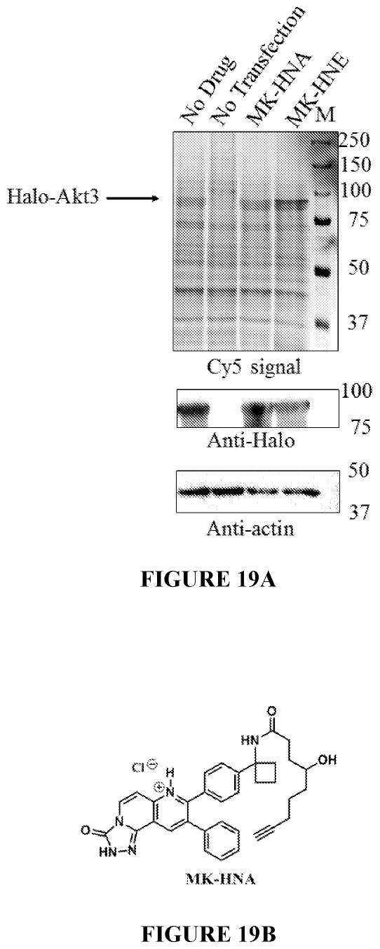

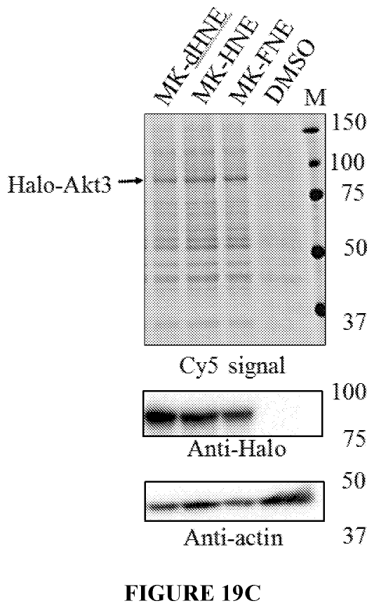

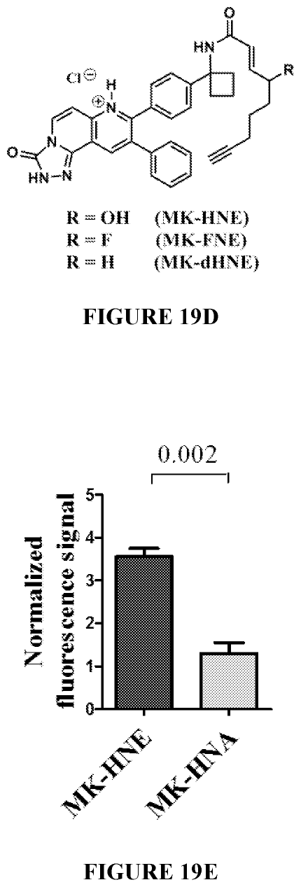

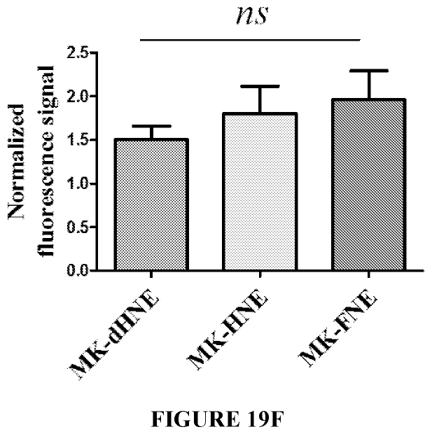

Data indicated that the enone function within MK-HNE is required for covalent binding to Akt3. To validate this hypothesis, an isosteric analog that lacks the enone moiety was created (FIG. 19B). This compound, called MK-HNA, would not be expected to interact covalently with Akt provided the mechanism of MK-HNE involves covalent conjugation between the enone and Akt3-cysteine(C119). Consistent with this hypothesis, no covalent labeling was observed when cells expressing Halo-Akt3 were treated with MK-HNA, but labeling was observed with MK-HNE (FIGS. 19A and 19E).

To understand some of the SAR of these covalent inhibitors, two other inhibitors based on MK-HNE, namely, MK-dHNE and MK-FNE were designed (FIG. 19D). All three inhibitors were able to elicit covalent labeling of Halo-Akt3 expressed in HEK293T cells (FIGS. 19C and 19F).

### Example 26—MK-HNE and Derivatives Inhibit Akt3 in a Time-Dependent Manner

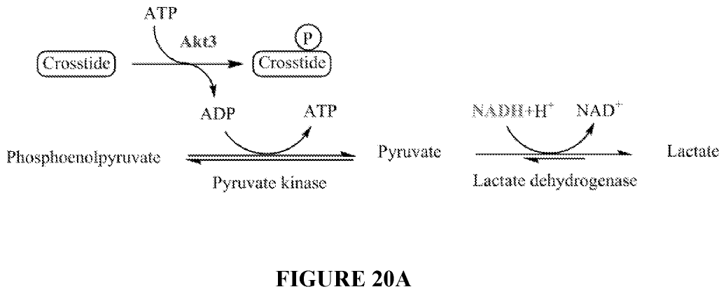

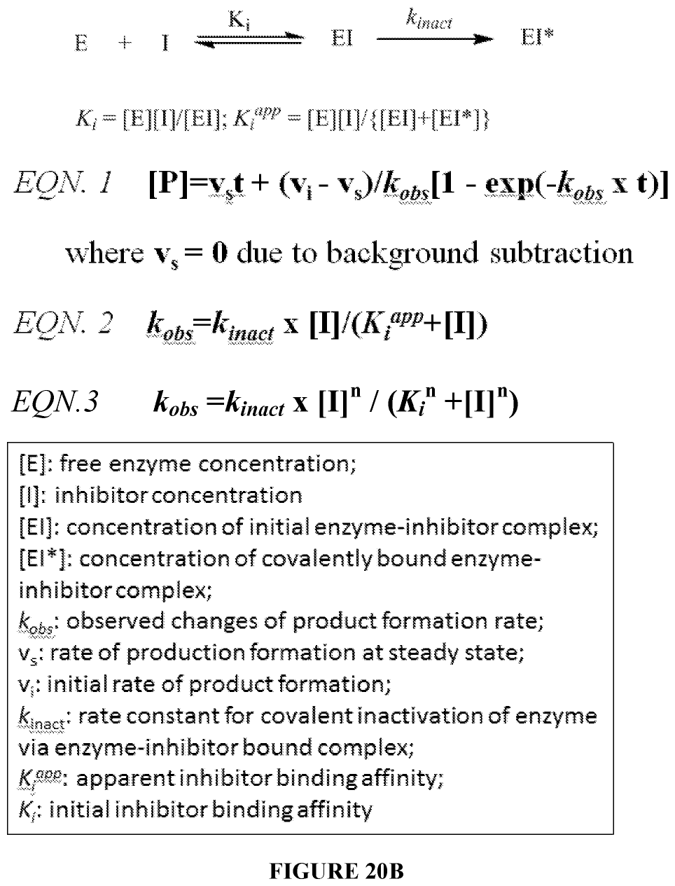

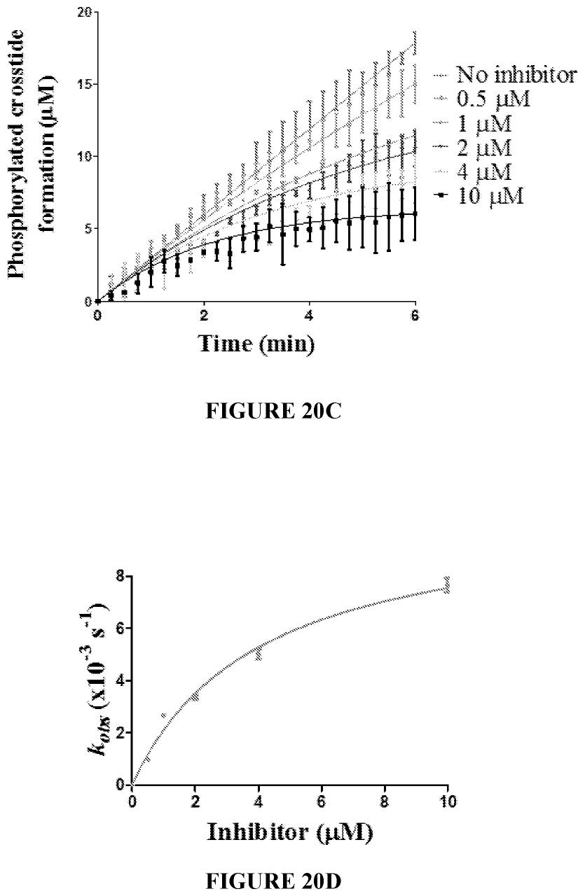

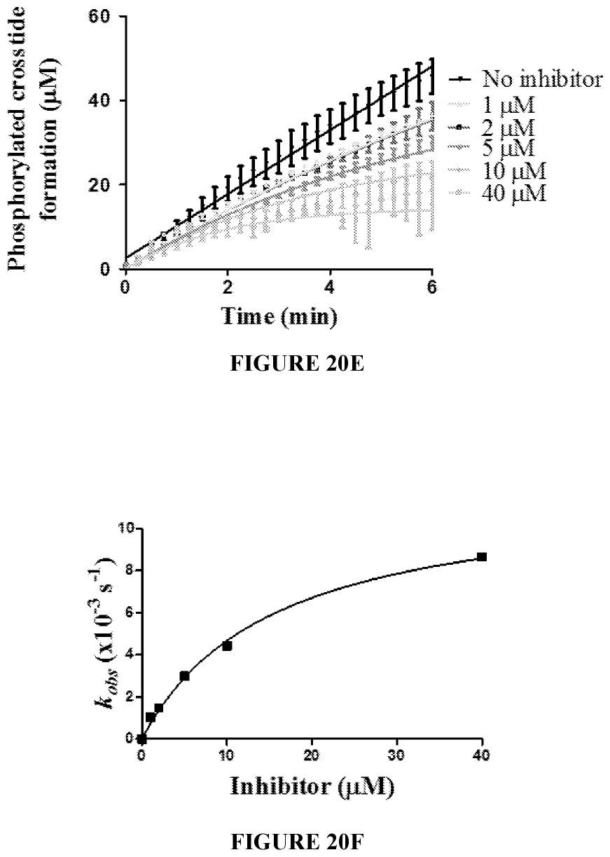

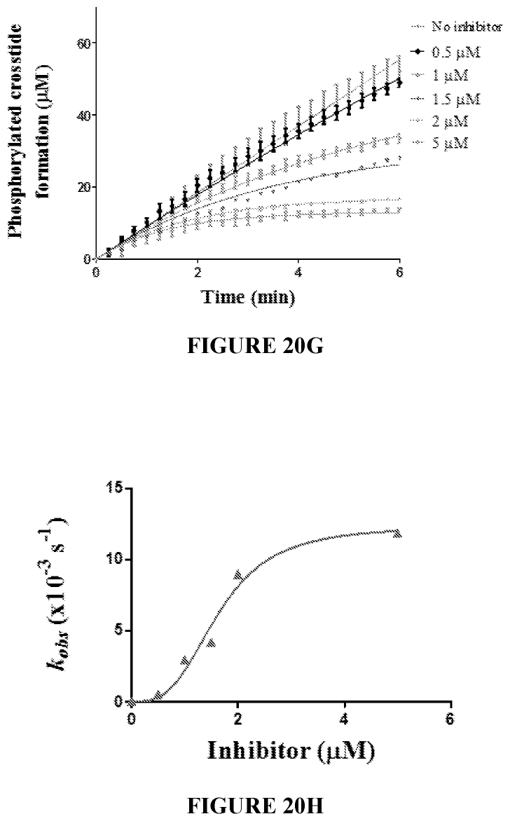

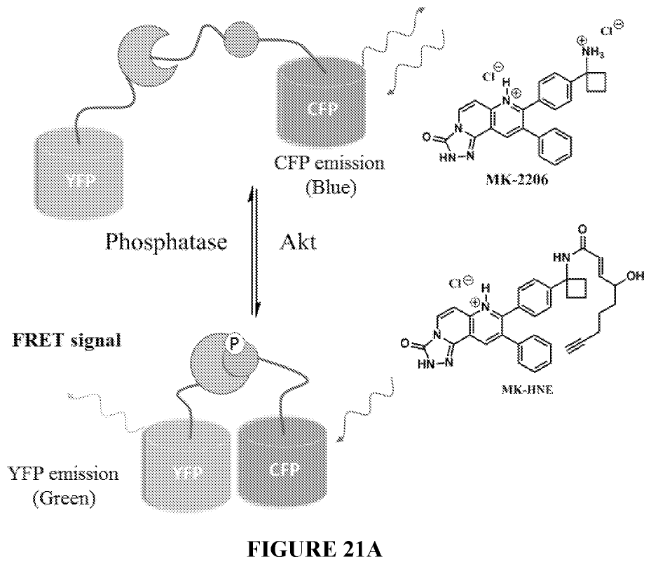

To gain a more quantitative understanding of the covalent inhibitors, inhibition of Akt3 by MK-HNE in vitro was investigated. A coupled spectrophotometric assay for kinase activity that employs phosphoenolpyruvate/pyruvate kinase to regenerate ATP from ADP formed upon each enzyme turnover was used (FIG. 20A). The pyruvate formed during ATP formation was then reduced to lactate by lactate dehydrogenase (LDH), in the process consuming one molecule of NADH per turnover. Loss of NADH was measured by decrease in A340 as a function of time. When Akt3 was treated with MK-HNE, a quintessential inhibition profile for a time-dependent inhibitor was observed.

Fitting the progress curves to a traditional time-dependent inhibition progress-curve equation gave a series of k_{obs} values (EQN. 1; FIG. 20B), that were fit to a Michaelis-Menten-type equation to derive k_{inact} and Kᵢ (EQN. 2; FIG. 20B). Repeating this analysis for the other inhibitors showed that MK-FNE (FIGS. 20G, 20H)>MK-HNE (FIGS. 20C, 20D)>MK-dHNE (FIGS. 20E and 20F). Pleasingly, MK-FNE had k_{inact}/Kᵢ close to 10⁴ M⁻¹S⁻¹ (Table 4). Interestingly, this inhibitor showed cooperative binding to Akt3 (Hill coefficient, n˜3). This is believed to be the first Akt-inhibitor characterized to have a cooperative inhibition profile.

TABLE 4
Parameters for MK-HNE, MK-dHNE, and MK-FNE

| | MK-HNE | MK-dHNE | MK-FNE |
|---|---|---|---|
| k_{inact} (s⁻¹) | 0.011 ± 0.001 | 0.012 ± 0.001 | 0.012 ± 0.002 |
| Kᵢ^{app} (mM) | 4.0 ± 0.5 | 15.5 ± 1.5 | 1.6 ± 0.2 |
| k_{inact}/Kᵢ^{app} (M⁻¹s⁻¹) | 2700 ± 340 | 770 ± 80 | 6700 ± 1200 |
| n | — | — | 3.2 |

Importantly, the improvement of MK-FNE over MK-HNE shows that optimization of the core structure to the protein of interest is possible and further shows that biologically meaningful inhibition rates can be simply achieved.

### Example 27—Unlike MK-2206, MK-HNE Inhibition of Akt is Persistent in Cells, Persistence Requires C119

One of the key benefits of covalent inhibition is the ability to uncouple pharmacokinetics from pharmacodynamics. This effect is achieved, because once irreversible inhibition occurs, an irreversible inhibitor will remain bound to its target post clearance of the unbound inhibitor. On the other hand, for a reversible inhibitor, as drug concentration drops, the drug is released from its target and inhibition is lost. This situation can be modeled in cell culture by removal of the drug from the media and measuring changes in activity as a function of time post drug removal. Akt inhibition was assayed using AKTAR (FIG. 21A). This fusion of two fluorescent proteins emits two different frequencies of light when excited with blue light, depending on the activity of Akt. When the construct is selectively phosphorylated by Akt, both blue and green light are emitted, due to fluorescence resonance energy transfer (FRET) from the CFP to the YFP. However, when the protein is not phosphorylated, emission is mainly in the blue channel. Thus the ratio of green to blue emission is a direct readout of Akt activity in live cells.

In this assay, specific isoforms of Akt were overexpressed to enable direct measurement of activity of that specific isoform in cells. Cells expressing Halo-Akt3 or Halo-Akt2 and AKTAR were treated with MK-2206 or MK-FNE for 24 hours. Both compounds elicited a decrease in green/blue fluorescence, consistent with Akt3 and Akt2 inhibition (FIGS. 21C-21F). Akt inhibition was maintained over the next 24 hours (during which time drug was still present, a flow chart for this experiment is found in FIG. 21A). After this time, each drug was removed from the media and cells were left to rest for a further 24 hours. After this time MK-2206-treated cells, Green/Blue levels had returned to those observed in untreated cells. However, for MK-HNE-treated cells, significant Akt3 inhibition was retained.

When the same experiment was performed in cells expressing Akt3(C119S) (electrophile-sensing defective but otherwise functional point mutant) (Long et al., Akt3 is a Privileged First Responder in Isozyme-Specific Electrophile Response,” Nat. Chem. Biol. 13(3):333-338 (2017), which is hereby incorporated by reference in its entirety), Akt inhibition was observed upon treatment with both MK-2206 and MK-HNE (FIGS. 21G, 21H). However, when MK-2206 and MK-HNE were removed from the media, no inhibition was observed in either case after 24 hours. This observation is consistent with covalent inhibition of Akt3 occurring through C119. Thus, MK-HNE inhibition of Akt2 and Akt3 is significantly different from MK-2206. This type of profile would be expected to lead to higher efficacy in animal models.

### Example 28—MK-FNE is Similarly Toxic to PTEN-Negative TNBC Cancer Lines as MK-2206

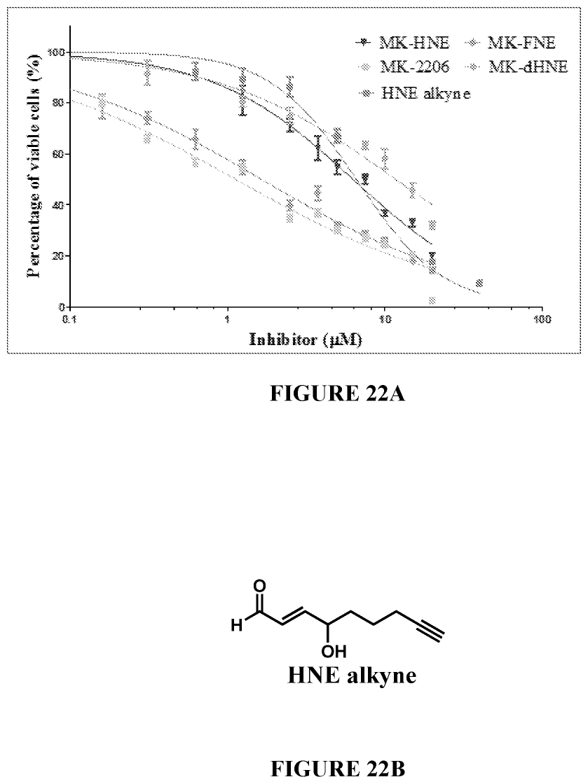

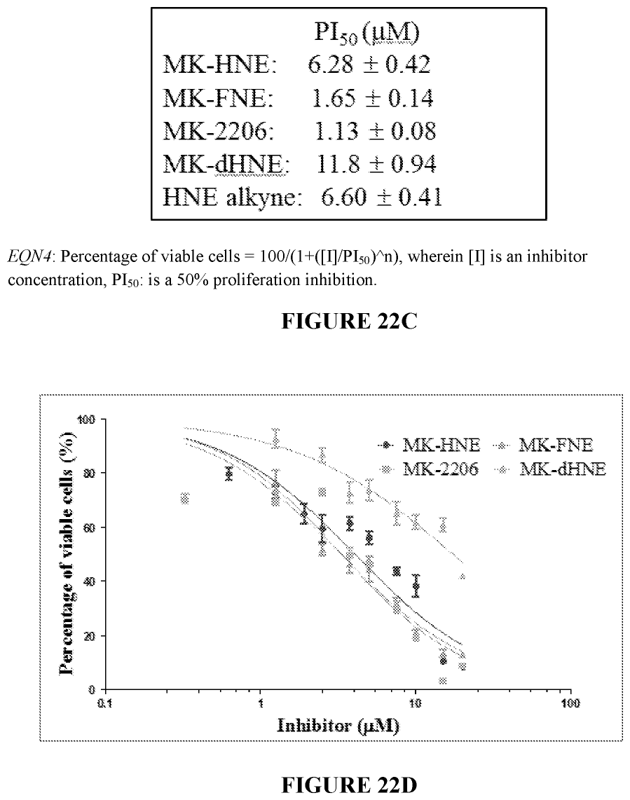

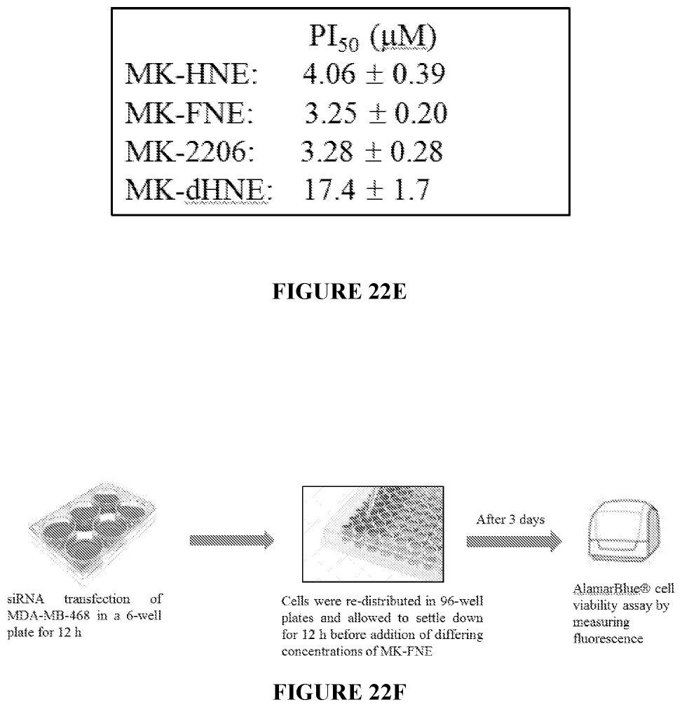

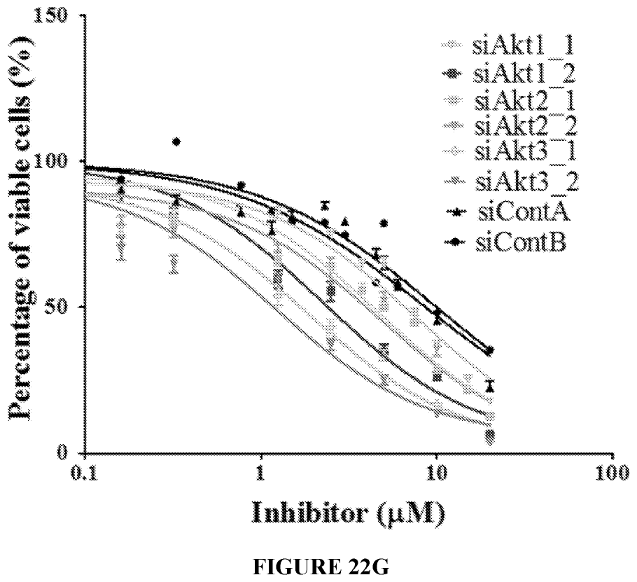

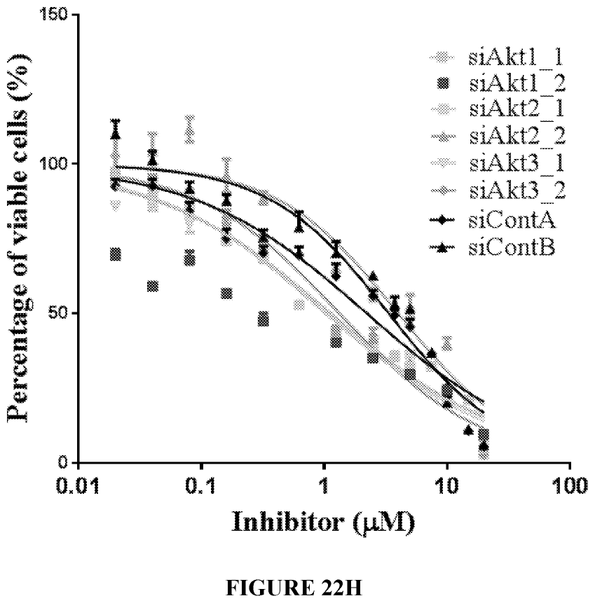

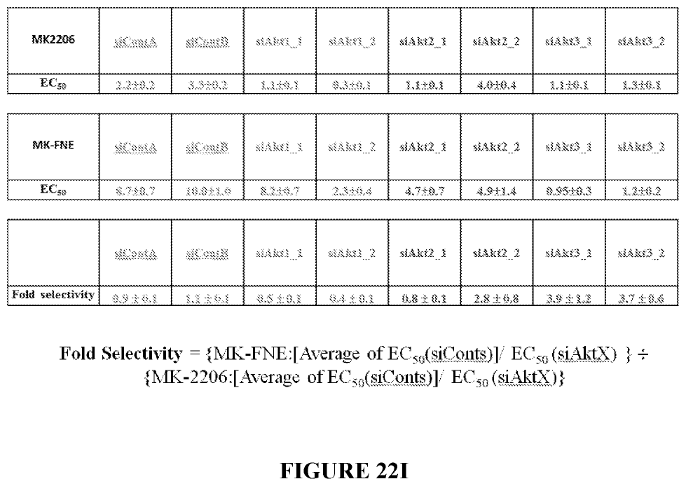

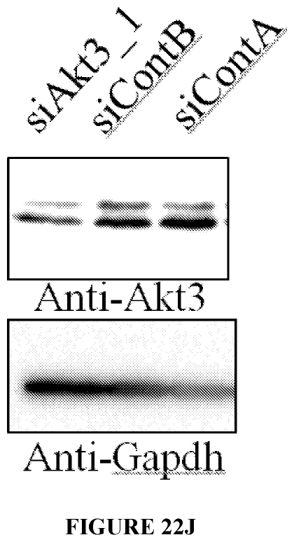

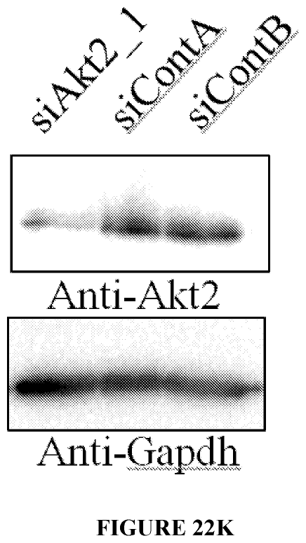

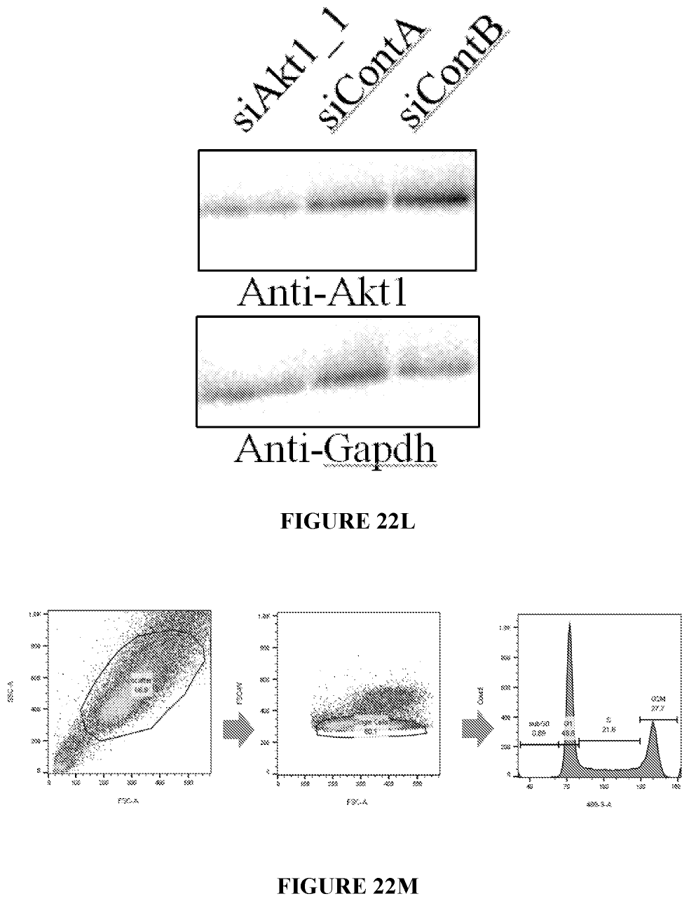

PTEN-null TNBC (triple-negative breast cancer) is a particularly deadly disease (Delaloge et al., “Targeting PI3K/AKT Pathway in Triple-Negative Breast Cancer,” The Lancet Oncology 18(10):1293-1294 (2017), which is hereby incorporated by reference in its entirety). Aggressiveness of PTEN-null TNBC is dependent on Akt activity. Several reports indicate that Akt2 and Akt3 are particularly important to these lines (Chin et al., “PTEN-Deficient Tumors Depend on AKT2 for Maintenance and Survival,” Cancer Discovery 4(8):942-955 (2014); Chin et al., “Targeting Akt3 Signaling in Triple-Negative Breast Cancer,” Cancer Research 74(3):964-973 (2014), which are hereby incorporated by reference in their entirety). In particular, upregulation of Akt3 expression is the major resistance mechanism of TNBCs and other breast cancers (BCs) against Akt inhibitor treatment (Stottrup et al., “Upregulation of AKT3 Confers Resistance to AKT Inhibitor MK-2206 in Breast Cancer,” Molecular Cancer Therapeutics 15(8): 1964-1974 (2016), which is hereby incorporated by reference in its entirety), indicating that Akt3 reliance is a dent in the armor of this dangerous disease. It is believed that these compounds would be toxic selectively to PTEN-null TNBC lines. Consistent with this logic, all of the MK-2206 derivatives were found to be toxic to MDA-MB-468 and BT-549 (both PTEN-null, TNBC lines; (FIGS. 22A-22F). MK-FNE was similarly potent to MK-2206, but MK-HNE and MK-dHNE were less effective than MK-2206. Importantly, the toxicity of the irreversible inhibitors mirrored the in vitro inhibition potencies against Akt3, namely MK-FNE>MK-HNE>MK-dHNE, consistent with selective Akt targeting by these compounds.

It was shown that knockdown of Akt3 (approximately 40-60%; (FIGS. 22J-22L) selectively sensitized cells to MK-FNE toxicity (around 8-fold) relative to control knockdown cells (FIGS. 22G-22I), whereas to a less extent for MK-2206 (2-3 fold). Akt1 or Akt2 knockdown (approximately 40-60%) was less effective (around 2-fold on average) at sensitizing the cell line to MK-FNE. Since depletion of the drug target typically sensitizes cells to drug treatment, this is good evidence that the molecules of the present invention inhibit Akt3, although clearly other Akt isoforms are also affected.

It is believed that if these compounds target Akt, they would exert the same effects on the cell cycle as other Akt inhibitors, i.e., the ATP-competitive inhibitors GDC-0068, GSK-69-0693 and the allosteric inhibitor MK-2206. On the other hand, if there were excessive off-target alkylation events elicited by the enone moiety, these MK-2206 derivatives would affect cells similarly to HNE. Using a standard flow cytometry protocol to gate single cells (FIG. 22M), it was shown that all Akt inhibitors locked cells in G1/G0 (Table 5). MK-HNE and MK-FNE behaved similarly to Akt inhibitors. HNE on the other hand caused S-phase arrest.

TABLE 5
Comparison of the PI₆₀ and PI₈₀ Data for MK-HNE, MK-FNE, and HNE Alkyne

| | | GDC- | GSK- | | | | | | | | |
|---|---|---|---|---|---|---|---|---|---|---|---|
| | | 0068 | 69063 | MK-2206 | | MK-HNE | | MK-FNE | | HNE alkyne | |
| | DMSO | (PI₆₀) | (PI₈₀) | (PI₆₀) | (PI₈₀) | (PI₆₀) | (PI₈₀) | (PI₆₀) | (PI₈₀) | (PI₆₀) | (PI₈₀) |
| G1/G0 | 42.3 | 52.6 | 47.7 | 41.8 | 50.2 | 41.7 | 49 | 47.0 | 58.9 | 40.0 | 28.5 |
| G2/M | 30.6 | 28.3 | 30.0 | 31.3 | 29.0 | 30.3 | 25.5 | 33.3 | 27.0 | 30.8 | 22 |
| S | 24.1 | 12.1 | 14.3 | 25.4 | 19.6 | 25.9 | 16.6 | 18.6 | 11.9 | 26.3 | 31.3 |
| subG0 | 0.7 | 1.64 | 0.92 | 0.3 | 0.3 | 0.4 | 5.5 | 0.5 | 0.8 | 3.7 | 18.2 |

### Example 29—Modification of MK-2206 with Bioactive Lipid Appendages Gives Unexpected Bioactivities that could be Beneficial for Drug Design

HNE-modification of Akt3 has different effects on the phosphorylation of Akt3 than MK-2206 binding to Akt3 does. One of the key differences is that phosphorylation of T305 is not perturbed by HNE whereas MK-2206 potently suppresses this modification (FIG. 23A). Western blot (FIGS. 23B-23D) and ratiometric ELISA (FIG. 23E) were used to compare how MK-HNE and MK-2206 affect T308 phosphorylation. MK-2206 lowered phospho-T305 (pT305) levels considerably, whereas similar to HNE modulation on Akt3 (Long et al., Akt3 is a Privileged First Responder in Isozyme-Specific Electrophile Response,” Nat. Chem. Biol. 13(3):333-338 (2017), which is hereby incorporated by reference in its entirety), MK-HNE treatment did not affect phosphorylation levels of Akt2 or Akt3. This indicated that pharmaceutical programs of MK-2206 and MK-HNE are likely different.

This is a critical observation because the effects of many covalent kinase inhibitors are nullified when a cancer cell develops a sensor cysteine mutation, and hence evolved resistance may be less efficient for natural RES-modified inhibitors.

Based on the above interesting observations, toxicity of the most potent compound MK-FNE and MK-2206 were screened against six other cultured cell lines (FIG. 23F). Four of these lines were breast cancer lines that are not PTEN-null TNBC, and the other two lines were fibroblast lines. Remarkably, it was found that MK-2206 is generally toxic to most lines, and it has little selectivity for PTEN-null TNBC lines. On the other hand, MK-FNE was significantly selective against PTEN-null TNBC lines. This was depicted graphically by calculating the PI₅₀(MK-FNE)/PI₅₀(MK-2206) (FIG. 23G). With one notable exception (i.e., TSE breast cancer cell lines), for all but PTEN-null TNBC lines, this value was greater than 2, and it rose above 8 in some lines.

An interesting note was a T47D line raising from the resistance to MK-2206 treatment (T47D MK-2206-res) was significantly less resistant to MK-FNE, hinting that MK-FNE may be able to overcome MK-2206 resistance.

Altogether these data indicated that MK-FNE has a better therapeutic window of tolerance than MK-2206. Importantly, based on the above data, it would be expected that the difference between MK-FNE and MK-2206 would increase in models where drug is cleared from the cells over time. To model this in cultured cells, MDA-MB-468 cells were treated with MK-2206 and MK-FNE for 48 hours. Then in half of the samples, drug was removed and replaced by fresh media. In the other half, drug was maintained (FIG. 23H). In MK-2206 cells, a significant regain of proliferation was observed, whereas in MK-FNE treated cells, there was no difference between withdrawal (wd) and continuous (cont) treatment.

Proposed mechanism for drug synergism with Akt isoform-specific knockdown is shown in FIG. 24. When target is depleted, the amount of drug required to achieve a pharmaceutically relevant dose is lowered. Knockdown of Akt3 sensitizes cells to a greater extent in response to MK-FNE treatment. This is in line with on-target Akt3 inhibition by MK-FNE.

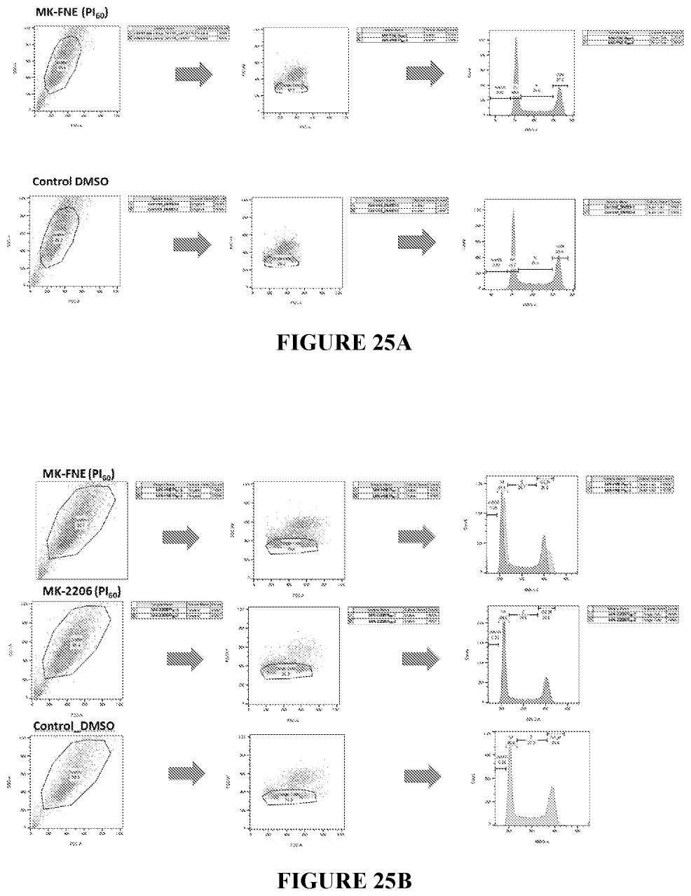

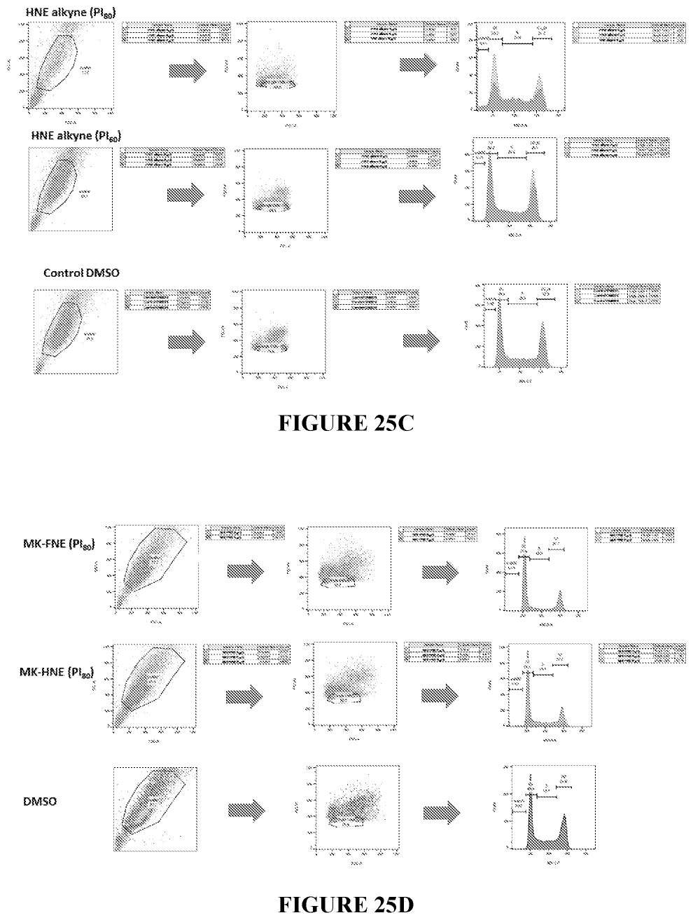

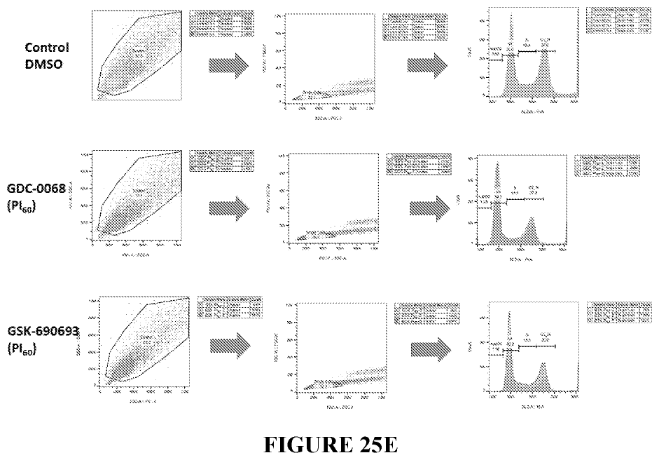

The results of cell cycle analysis using flow cytometry are shown in FIGS. 25A-25E. MK-FNE and MK-HNE caused G1 arrest, similar to MK-2206 and other Akt inhibitors. HNE causes loss of G1. Although the effects of HNE are cell-type dependent (Chaudhary et al., “4-Hydroxynonenal Induces G2/M Phase Cell Cycle Arrest by Activation of the Ataxia Telangiectasia Mutated and Rad3-related Protein (ATR)/Checkpoint Kinase 1 (Chk1) Signaling Pathway,” J. Biol. Chem. 288(28):20532-20546 (2013), which is hereby incorporated by reference in its entirety), clearly in Akt3-dependent lines, MK-FNE/MK-HNE's effects are more similar to MK-2206 than HNE. However, the data above show that effects of MK-FNE/MK-HNE on Akt3 are similar to those seen upon HNE labeling Akt3 rather than MK-2206 binding to Akt3. Thus on an Akt3-specific level, MK-HNE/MK-FNE behave like HNE, but in whole cells, they behave like a classic Akt inhibitor. These data give further evidence that Akt3 is the principal target of MK-FNE/MK-HNE and that these inhibitors are not hitting other targets of HNE.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A compound of Formula (I): 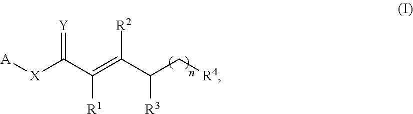
wherein
A is a reversible pan-Akt inhibitor or a radical thereof;
X is optional, and, if present, is NH, O, or S;
Y is O or S;
R¹ is H, halogen, NO₂, CN, C₁₋₆ alkyl, or aryl;
R² is H, halogen, NO₂, CN, C₁₋₆ alkyl, or aryl;
R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;
R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;
R⁵ is H, C₁₋₆ alkyl, or aryl;
R⁶ is H, C₁₋₆ alkyl, or aryl; and
n is 0, 1, 2, 3, 4, or 5;
or an oxide thereof, a pharmaceutically acceptable salt thereof, a solvate thereof, or a prodrug thereof.

2. The compound according to claim 1, wherein X is NH, O, or S.

3. The compound according to claim 2, wherein A is a radical of a compound selected from the group consisting of MK-2206, GSK690693, and Akt-I-1,2 (2-[4-(3-phenylquinoxalin-2-yl)phenyl]propan-2-amine) (CAS #473382-48-8).

4. The compound according to claim 1, wherein A is a radical of inhibitor VIII.

5. The compound according to claim 2, wherein the compound of Formula (I) is a cis isomer.

6. The compound according to claim 2, wherein the compound of Formula (I) is a trans isomer.

7. The compound according to claim 2, wherein the compound of Formula (I) has the Formula (Ia): 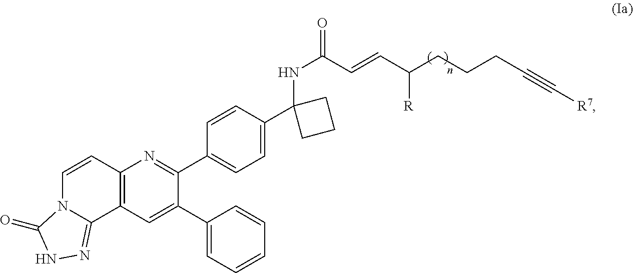
wherein
R is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;
R⁷ is H, CN, CF₃, CO₂H, SiMe₃, C₁₋₆ alkyl, or C₁₋₆ alkoxy; and
m is 0, 1, 2, or 3.

8. The compound according to claim 2, wherein the compound of Formula (I) is selected from the group consisting of:

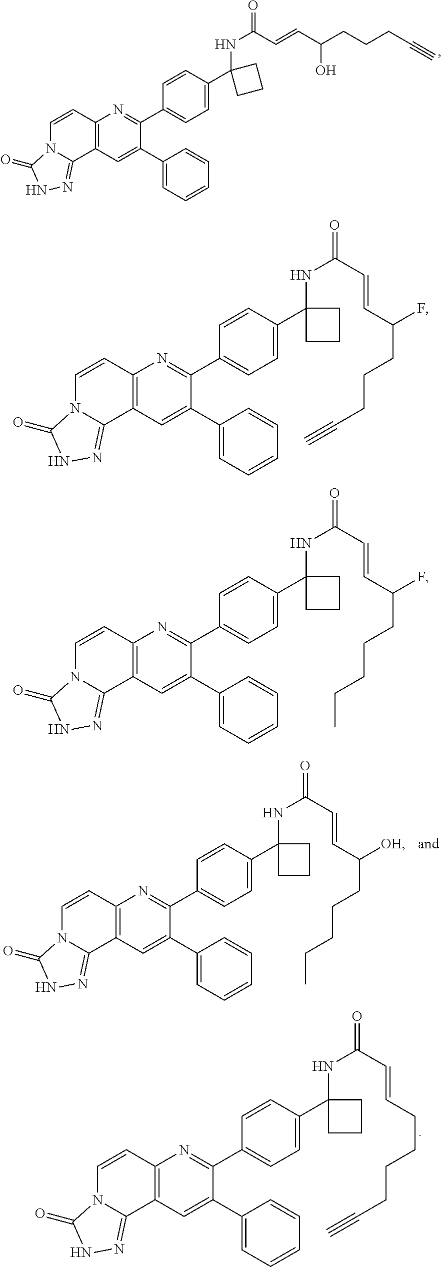 [SMILES: C#CCCCC(O)/C=C/C(=O)NC1(c2ccc(-c3nc4ccn5c(=O)[nH]nc5c4cc3-c3ccccc3)cc2)CCC1 ; C#CCCCC(F)/C=C/C(=O)NC1(c2ccc(-c3nc4ccn5c(=O)[nH]nc5c4cc3-c3ccccc3)cc2)CCC1 ; CCCCCC(F)/C=C/C(=O)NC1(c2ccc(-c3nc4ccn5c(=O)[nH]nc5c4cc3-c3ccccc3)cc2)CCC1 ; CCCCCC(O)/C=C/C(=O)NC1(c2ccc(-c3nc4ccn5c(=O)[nH]nc5c4cc3-c3ccccc3)cc2)CCC1 ; C#CCCCC/C=C/C(=O)NC1(c2ccc(-c3nc4ccn5c(=O)[nH]nc5c4cc3-c3ccccc3)cc2)CCC1]

9. The compound according to claim 2, wherein the compound of Formula (I) is selected from the group consisting of:

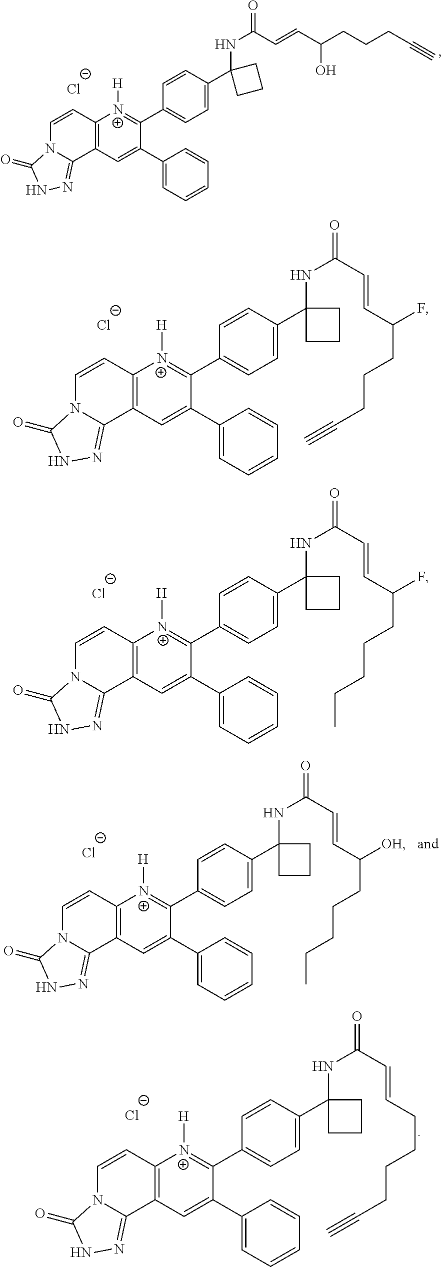

10. A pharmaceutical composition comprising a therapeutically effective amount of the compound according to claim 2 and a pharmaceutically acceptable carrier.

11. A method of treating cancer, said method comprising:
administering, to a subject, a compound of the Formula (I): 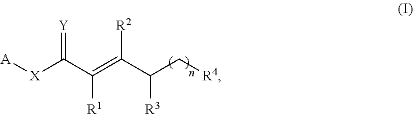
wherein
A is a reversible pan-Akt inhibitor or a radical thereof;
X is optional, and, if present, is NH, O, or S;
Y is O or S;
R¹ is H, halogen, NO₂, CN, C₁₋₆ alkyl, or aryl;
R² is H, halogen, NO₂, CN, C₁₋₆ alkyl, or aryl;
R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;
R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;
R⁵ is H, C₁₋₆ alkyl, or aryl;
R⁶ is H, C₁₋₆ alkyl, or aryl; and
n is 0, 1, 2, 3, 4, or 5;
or an oxide thereof, a pharmaceutically acceptable salt thereof, a solvate thereof, or a prodrug thereof,
under conditions effective to treat cancer in the subject.

12. The method of claim 11, wherein X is NH, O, or S.

13. The method of claim 12 further comprising:
selecting a subject having cancer mediated by Akt2 or Akt3, wherein the selected subject receives the administered compound.

14. The method of claim 12, wherein A is a radical of a compound selected from the group consisting of MK-2206, GSK690693, and Akt-I-1,2 (2-[4-(3-phenylquinoxalin-2-yl)phenyl]propan-2-amine) (CAS #473382-48-8).

15. The method of claim 11, wherein A is a radical of inhibitor VIII.

16. The method of claim 12, wherein the compound of Formula (I) is a cis isomer.

17. The method of claim 12, wherein the compound of Formula (I) is a trans isomer.

18. The method of claim 12, wherein the compound of Formula (I) has the Formula (Ia): 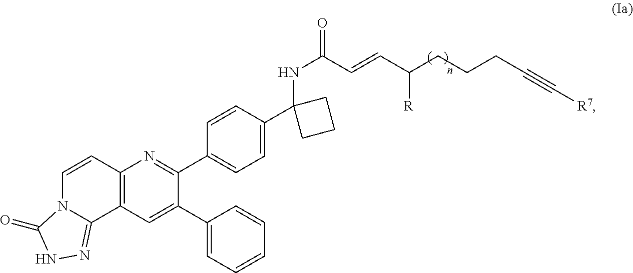
wherein
R is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;
R⁷ is H, CN, CF₃, CO₂H, SiMe₃, C₁₋₆ alkyl, or C₁₋₆ alkoxy; and
m is 0, 1, 2, or 3.

19. The method of claim 12, wherein the compound of Formula (I) is selected from the group consisting of:

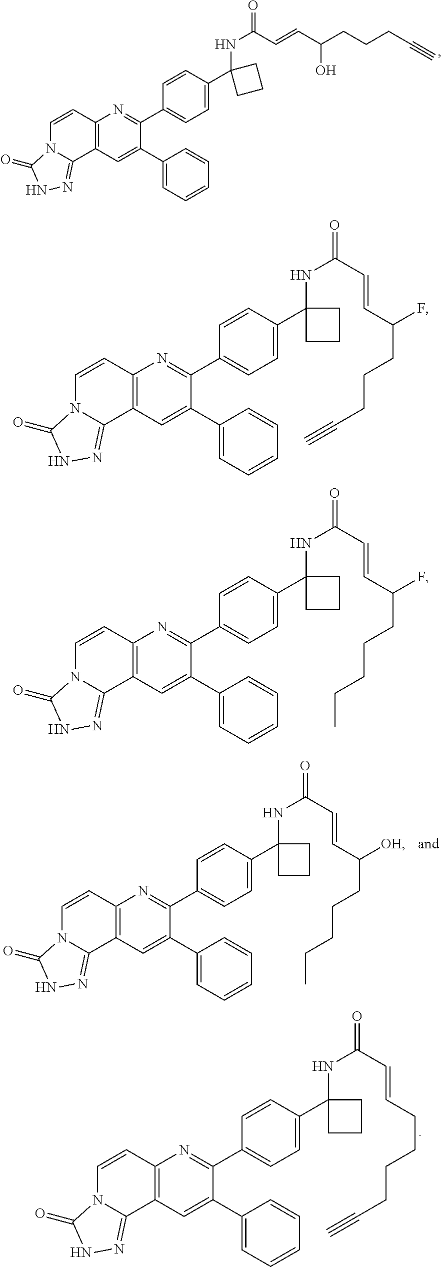 [SMILES: C#CCCCC(O)/C=C/C(=O)NC1(c2ccc(-c3nc4ccn5c(=O)[nH]nc5c4cc3-c3ccccc3)cc2)CCC1 ; C#CCCCC(F)/C=C/C(=O)NC1(c2ccc(-c3nc4ccn5c(=O)[nH]nc5c4cc3-c3ccccc3)cc2)CCC1 ; CCCCCC(F)/C=C/C(=O)NC1(c2ccc(-c3nc4ccn5c(=O)[nH]nc5c4cc3-c3ccccc3)cc2)CCC1 ; CCCCCC(O)/C=C/C(=O)NC1(c2ccc(-c3nc4ccn5c(=O)[nH]nc5c4cc3-c3ccccc3)cc2)CCC1 ; C#CCCCC/C=C/C(=O)NC1(c2ccc(-c3nc4ccn5c(=O)[nH]nc5c4cc3-c3ccccc3)cc2)CCC1]

20. The method of claim 12, wherein the compound of Formula (I) is selected from the group consisting of:

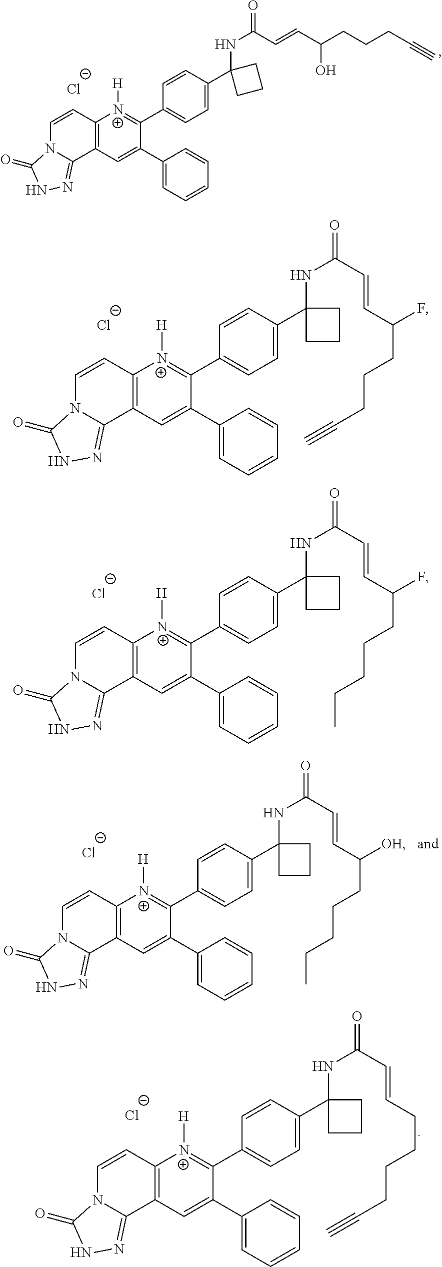

21. The method of claim 12, wherein cancer is treated, said cancer being selected from the group consisting of breast cancer, prostate cancer, colon cancer, lung cancer, and ovarian cancer.

22. The method according to claim 12, wherein the said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

23. A method of inhibiting pan-Akt in a cell or a tissue, said method comprising:
providing a compound of Formula (I): 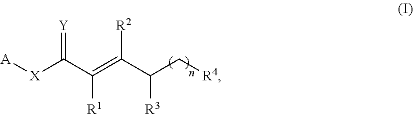
wherein
A is a reversible pan-Akt inhibitor or a radical thereof;
X is optional, and, if present, is NH, O, or S;
Y is O or S;
R¹ is H, halogen, NO₂, CN, C₁₋₆ alkyl, or aryl;
R² is H, halogen, NO₂, CN, C₁₋₆ alkyl, or aryl;
R³ is selected from the group consisting of H, halogen, C₁₋₆ alkyl, C₃₋₆ cycloalkyl, OC₁₋₆ alkyl, OH, NO₂, N₃, NR⁵R⁶, CN, CF₃, and CO₂H;
R⁴ is H, OH, NR⁵R⁶, SiMe₃, C₁₋₆ alkyl, C₂₋₆ alkenyl, or C₂₋₆ alkynyl, wherein C₁₋₆ alkyl, C₂₋₆ alkenyl, and C₂₋₆ alkynyl can be optionally substituted from 1 to 3 times with a substituent selected independently at each occurrence thereof from the group consisting of H, OH, NR⁵R⁶, C₁₋₆ alkyl, C₂₋₆ alkenyl, C₂₋₆ alkynyl, and C₁₋₆ alkoxy;
R⁵ is H, C₁₋₆ alkyl, or aryl;
R⁶ is H, C₁₋₆ alkyl, or aryl; and
n is 0, 1, 2, 3, 4, or 5;
or an oxide thereof, a pharmaceutically acceptable salt thereof, a solvate thereof, or a prodrug thereof; and
contacting a cell or tissue with the compound under conditions effective to inhibit pan-Akt.

24. The method of claim 23, wherein X is NH, O, or S.

25. The method of claim 24, wherein A is a radical of a compound selected from the group consisting of MK-2206, GSK690693, and Akt-I-1,2 (2-[4-(3-phenylquinoxalin-2-yl)phenyl]propan-2-amine) (CAS #473382-48-8).

26. The method of claim 23, wherein A is a radical of inhibitor VIII.

27. The method of claim 24, wherein the compound of Formula (I) is a cis isomer.

28. The method of claim 24, wherein the compound of Formula (I) is a trans isomer.

29. The method of claim 24, wherein the compound of Formula (I) has the Formula (Ia): 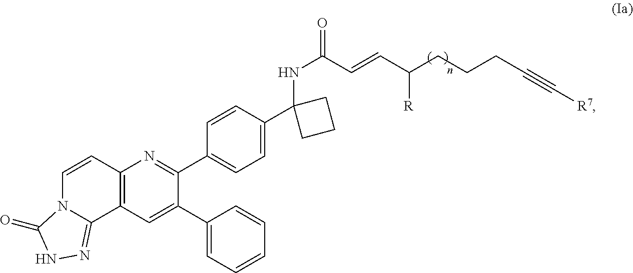
wherein
R⁷ is H, CN, CF₃, CO₂H, SiMe₃, C₁₋₆ alkyl, or C₁₋₆ alkoxy; and
m is 0, 1, 2, or 3.

30. The method of claim 24, wherein the compound of Formula (I) is selected from the group consisting of:

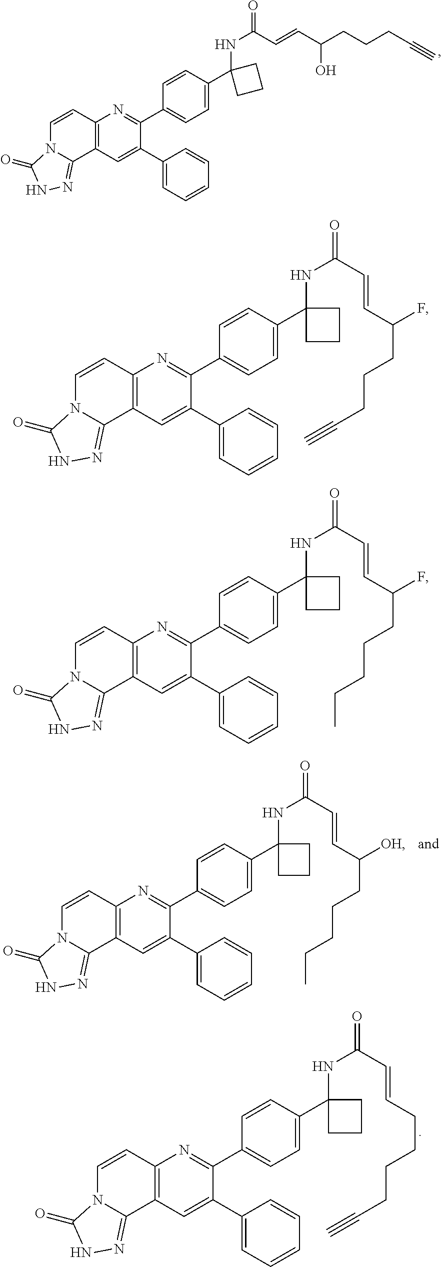 [SMILES: C#CCCCC(O)/C=C/C(=O)NC1(c2ccc(-c3nc4ccn5c(=O)[nH]nc5c4cc3-c3ccccc3)cc2)CCC1 ; C#CCCCC(F)/C=C/C(=O)NC1(c2ccc(-c3nc4ccn5c(=O)[nH]nc5c4cc3-c3ccccc3)cc2)CCC1 ; CCCCCC(F)/C=C/C(=O)NC1(c2ccc(-c3nc4ccn5c(=O)[nH]nc5c4cc3-c3ccccc3)cc2)CCC1 ; CCCCCC(O)/C=C/C(=O)NC1(c2ccc(-c3nc4ccn5c(=O)[nH]nc5c4cc3-c3ccccc3)cc2)CCC1 ; C#CCCCC/C=C/C(=O)NC1(c2ccc(-c3nc4ccn5c(=O)[nH]nc5c4cc3-c3ccccc3)cc2)CCC1]

31. The method of claim 24, wherein the compound of Formula (I) is selected from the group consisting of: [chemical structure]
